(19) United States
(12) Patent Application Publication
(10) Pub. No.: US 2023/0143779 A1
(43) Pub. Date: May 11, 2023

(54) ORGANIC SOLID-STATE LASER, COMPOUND AND USE THEREOF

(71) Applicants: KOALA Tech Inc., Fukuoka-shi, Fukuoka (JP); CENTRE NATIONAL DE LA RECHERCHE SCIENTIFIQUE, Paris (FR); SORBONNE UNIVERSITE, Paris (FR); INDUSTRY-ACADEMIC COOPERATION FOUNDATION, YONSEI UNIVERSITY, Seoul (KR); KYUSHU UNIVERSITY, NATIONAL UNIVERSITY CORPORATION, Fukuoka-shi, Fukuoka (JP)

(72) Inventors: Anthony D'ALEO, Marseille (FR); Jean Charles Maurice RIBIERRE, Fukuoka-shi, Fukuoka (JP); Fatima BENCHEIKH, Fukuoka-shi, Fukuoka (JP); Virginie Simone Francoise PLACIDE, Fukuoka-shi, Fukuoka (JP); Masashi MAMADA, Fukuoka-shi, Fukuoka (JP); Chihaya ADACHI, Fukuoka-shi, Fukuoka (JP)

(21) Appl. No.: 17/904,247

(22) PCT Filed: Feb. 9, 2021
(86) PCT No.: PCT/JP2021/004789

§ 371 Date: Aug. 15, 2022

(30) Foreign Application Priority Data
Feb. 13, 2020 (JP) 2020-022289

(51) Int. Cl. H01S 3/16; C07D 285/14; C07D 249/18; C07D 417/14; C07F 7/08; C07D 513/04; C07D 293/10; C09K 11/06

(52) U.S. Cl. CPC H01S 3/16; C07D 285/14; C07D 249/18; C07D 417/14; C07F 7/0812; C07D 513/04; C07D 293/10; C09K 11/06; C09K 2211/1018

(57) ABSTRACT

A compound represented by the formula (1) has excellent lasing properties. G¹ and G² are H or substituent; FL¹ and FL² are represented by the formula (2); BT is represented by the formula (4); and n1, n2 and m are 1 to 5.

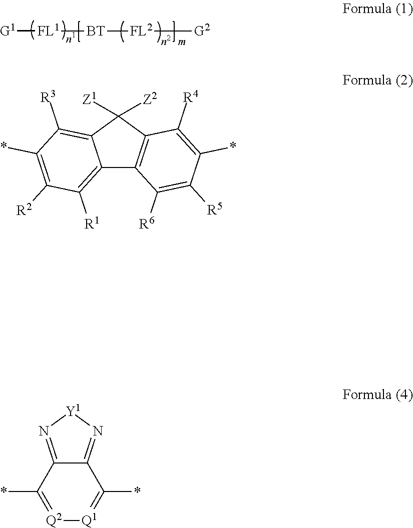

### TECHNICAL FIELD

The present invention relates to an organic solid-state laser, a novel compound and use of the compound as an emitter in an organic solid-state laser.

### BACKGROUND ART

Research for developing an organic solid-state laser having a low laser oscillation threshold has been actively conducted. In order to realize such an organic solid-state laser, it is necessary to develop organic compounds having excellent lasing properties. For this reason, various organic compounds have been synthesized and their lasing properties have been investigated. Non-Patent Document 1 reports that bisstilbene derivatives such as BSBCz exhibit a low ASE (Amplified Spontaneous Emission) threshold and they are excellent organic laser materials. However, the number of useful organic laser materials is still small.

### CITATION LIST

### Non Patent Literature

NPL 1: Appl. Phys. Lett. 2005, 86, 071110

### SUMMARY OF INVENTION

### Technical Problem

An object of the present invention is to provide a new organic laser material and an organic solid-state laser using the material.

### Solution to Problem

As a result of earnest investigations, the inventors have found that a group of compounds with a particular structure have excellent lasing properties. Thus, the inventors have provided the following invention:

[1] An organic solid-state laser containing a compound represented by the following formula (1):

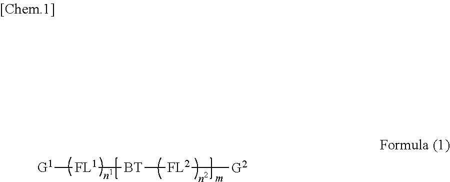

wherein:

G¹ and G² each independently represent a hydrogen atom or a substituent;

FL¹ and FL² each independently represent a linking group represented by the following formula (2) or formula (3):

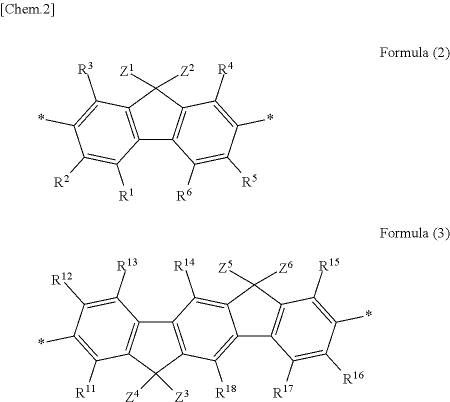

wherein R¹ to R⁶, R¹¹ to R¹⁸ and Z¹ to Z⁶ each independently represent a hydrogen atom or a substituent, R¹ and R², R⁵ and R⁶, R¹² and R¹³, and R¹⁶ and R¹⁷ may be bonded together to form a ring, and each * represents a bonding site;
BT represents a linking group represented by the following formula (4) or formula (5):

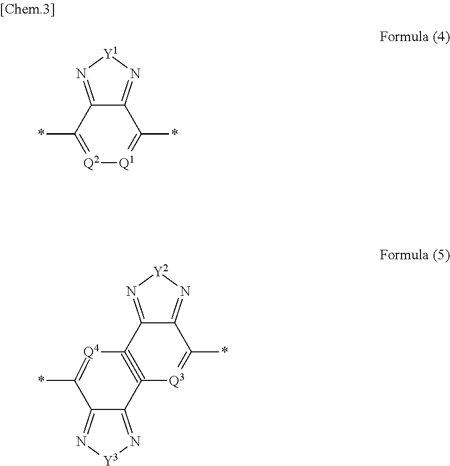

wherein Y¹ represents an oxygen atom, a sulfur atom or N-T¹, Y² represents an oxygen atom, a sulfur atom or N-T², Y³ represents an oxygen atom, a sulfur atom or N-T³, Q¹ represents a nitrogen atom or C—X¹, Q² represents a nitrogen atom or C—X², Q³ represents a nitrogen atom or C—X¹, Q⁴ represents a nitrogen atom or C—X⁴, T¹ to T³ each independently represent a substituted or unsubstituted alkyl group, X¹ to X⁴ each independently represent a hydrogen atom, a substituted or unsubstituted alkyl group, a substituted or unsubstituted alkoxy group or a halogen atom, or X¹ and X² may be bonded together to form a ring, and each * represents a bonding site;
n1, n2 and m each independently represent an integer of from 1 to 5; rightmost n2 may be 0; and when m is an integer of from 2 to 5 and rightmost n2 is 0, then n1 may be 0;
when m is two or more, then two or more instances of BT may be the same or different, two or more instances of FL² may be the same or different, and two or more instances of n2 may be the same or different;
when n1 is two or more, then two or more instances of FL¹ may be the same or different; and
when n2 is two or more, then two or more instances of FL² may be the same or different.

[2] The organic solid-state laser according to [1], wherein BT is a linking group represented by the formula (4).

[3] The organic solid-state laser according to [1], wherein BT is a linking group represented by the formula (5).

[4] The organic solid-state laser according to [2], wherein BT is a linking group represented by the following formula (4a):

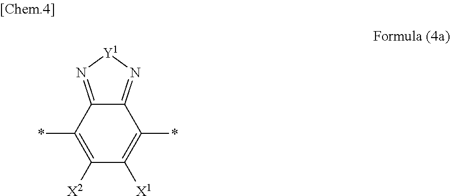

wherein Y¹ represents an oxygen atom, a sulfur atom or N-T¹, T¹ represents a substituted or unsubstituted alkyl group, X¹ and X² each independently represent a hydrogen atom, a substituted or unsubstituted alkyl group, a substituted or unsubstituted alkoxy group or a halogen atom, or X¹ and X² may be bonded together to form a ring, and each * represents a bonding site.

[5] The organic solid-state laser according to [4], wherein BT is a linking group represented by the following formula (4b):

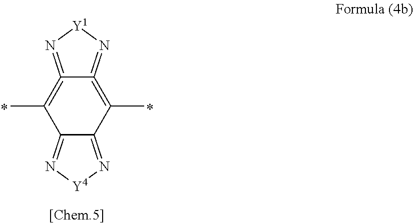

wherein Y¹ represents an oxygen atom, a sulfur atom or N-T¹, Y⁴ represents an oxygen atom, a sulfur atom or N-T⁴, T¹ and T⁴ each independently represent a substituted or unsubstituted alkyl group, and each * represents a bonding site.

[6] The organic solid-state laser according to [4], wherein BT is a linking group represented by the following formula (4c):

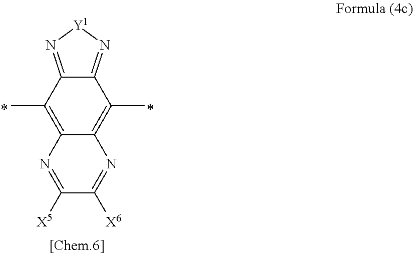

wherein Y¹ represents an oxygen atom, a sulfur atom or N-T¹, T¹ represent a substituted or unsubstituted alkyl group, X⁵ and X⁶ each independently represent a hydrogen atom a substituted or unsubstituted alkyl group, X⁵ and X⁶ can be bonded together to form a ring, and each * represents a bonding site.

[7] The organic solid-state laser according to [3], wherein BT is a linking group represented by the following formula (5a):

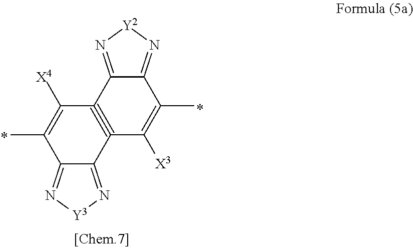

wherein Y² represents an oxygen atom, a sulfur atom or N-T², Y³ represents an oxygen atom, a sulfur atom or N-T³, T² and T³ each independently represent a substituted or unsubstituted alkyl group, X³ and X⁴ each independently represent a hydrogen atom, a substituted or unsubstituted alkyl group, a substituted or unsubstituted alkoxy group or a halogen atom, and each * represents a bonding site.

[8] The organic solid-state laser according to [1], wherein BT is each independently selected from the group consisting of the following BT1 to BT12:

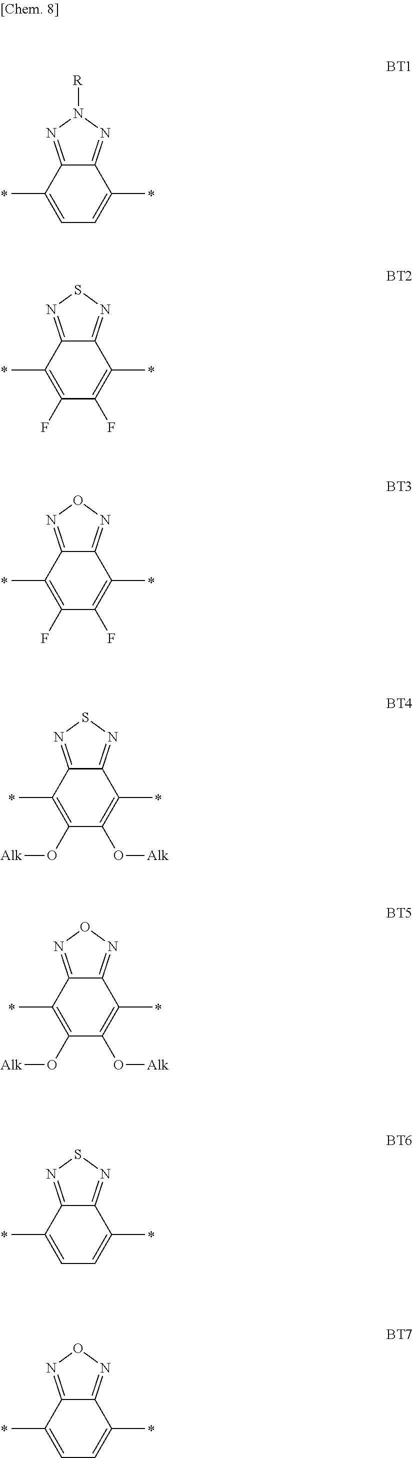

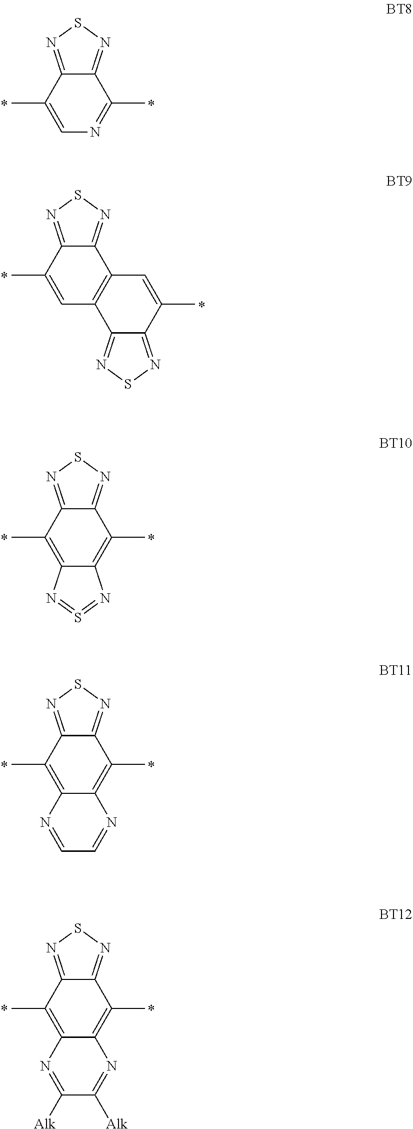

wherein R and Alk each independently represent a substituted or unsubstituted alkyl group and each * represents a bonding site.

[9] The organic solid-state laser according to any one of [1] to [8], wherein FL¹ and FL² are each independently a linking group represented by the formula (2).

[10] The organic solid-state laser according to any one of [1] to [8], wherein FL¹ and FL² are each independently a linking group represented by the formula (3).

[11] The organic solid-state laser according to any one of [1] to [10], wherein Z¹ to Z⁶ each independently represent a substituted or unsubstituted alkyl group.

[12] The organic solid-state laser according to any one of [1] to [11], wherein R¹ to R⁶ and R¹¹ to R¹⁸ are a hydrogen atom.

[13] The organic solid-state laser according to any one of [1] to [12], wherein G¹ and G² are each independently a substituted or unsubstituted aryl group or a substituted or unsubstituted diarylamino group.

[14] The organic solid-state laser according to any one of [1] to [13], wherein the compound is represented by the following formula (1a):

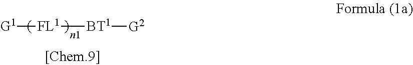

wherein G¹ and G² each independently represent a hydrogen atom or a substituent, FL¹ represents a linking group represented by the formula (2) or formula (3), BT¹ represents a linking group represented by the formula (4) or formula (5), and n1 is an integer of from 1 to 5.

[15] The organic solid-state laser according to any one of [1] to [13], wherein the compound is represented by the following formula (1b):

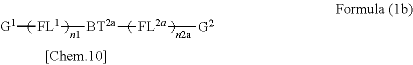

wherein G¹ and G² each independently represent a hydrogen atom or a substituent, FL¹ and FL^{2a} each independently represent a linking group represented by the formula (2) or formula (3), BT^{2a} represents a linking group represented by the formula (4) or formula (5), and n1 and n2a are each independently an integer of from 1 to 5.

[16] The organic solid-state laser according to any one of [1] to [13], wherein the compound is represented by the following formula (1c):

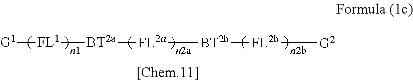

wherein G¹ and G² each independently represent a hydrogen atom or a substituent, FL¹, FL^{2a} and FL^{2b} each independently represent a linking group represented by the formula (2) or formula (3), BT^{2a} and BT^{2b} each independently represent a linking group represented by the formula (4) or formula (5), n1 and n2a are each independently an integer of from 1 to 5, and n2b is an integer of from 0 to 5.

[17] The organic solid-state laser according to any one of [1] to [13], wherein the compound is represented by the following formula (1d):

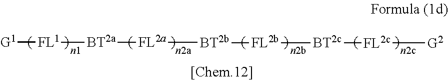

wherein G¹ and G² each independently represent a hydrogen atom or a substituent, FL¹, FL^{2a}, FL^{2b} and FL^{2c} each independently represent a linking group represented by the formula (2) or formula (3), BT^{2a}, BT^{2b} and BT^{2c} each independently represent a linking group represented by the formula (4) or formula (5), n1, n2a and n2b are each independently an integer of from 1 to 5, and n2c is an integer of from 0 to 5.

[18] The organic solid-state laser according to any one of [1] to [13], wherein the compound is represented by the following formula (1e):

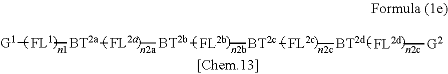

wherein G¹ and G² each independently represent a hydrogen atom or a substituent, FL¹, FL^{2a}, FL^{2b}, FL^{2c} and FL^{2d} each independently represent a linking group represented by the formula (2) or formula (3), BT^{2a}, BT^{2b}, BT^{2c} and BT^{2d} each independently represent a linking group represented by the formula (4) or formula (5), n1, n2a, n2b and n2c are each independently an integer of from 1 to 5, and n2d is an integer of from 0 to 5.

[19] The organic solid-state laser according to any one of [1] to [13], wherein the compound is represented by the following formula (1f):

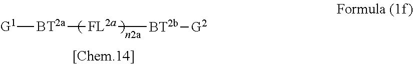

wherein, G¹ and G² each independently represent a hydrogen atom or a substituent, FL^{2a} represents a linking group represented by the formula (2) or formula (3), BT^{2a} and BT^{2b} each independently represent a linking group represented by the formula (4) or formula (5), and n2a is an integer of from 1 to 5.

[20] The organic solid-state laser according to any one of [1] to [13], wherein the compound is represented by the following formula (1g):

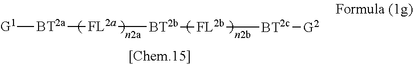

wherein G¹ and G² each independently represent a hydrogen atom or a substituent, FL^{2a} and FL^{2b} each independently represent a linking group represented by the formula (2) or formula (3), BT^{2a}, BT^{2b} and BT^{2c} each independently represent a linking group represented by the formula (4) or formula (5), and n2a and n2b are each independently an integer of from 1 to 5.

[21] The organic solid-state laser according to any one of [1] to [13], wherein the compound is represented by the following formula (1h):

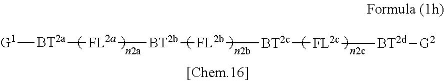

wherein G¹ and G² each independently represent a hydrogen atom or a substituent, FL^{2a}, FL^{2b} and FL^{2c} each independently represent a linking group represented by the formula (2) or formula (3), BT^{2a}, BT^{2b}, BT^{2c} and BT^{2d} each independently represent a linking group represented by the formula (4) or formula (5), and n2a, n2b and n2c are each independently an integer of from 1 to 5.

[22] A compound represented by the following formula (1):

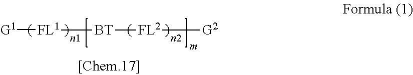

wherein:

G¹ and G² each independently represent a hydrogen atom or a substituent;

FL¹ and FL² are each independently a linking group represented by the following formula (2) or formula (3):

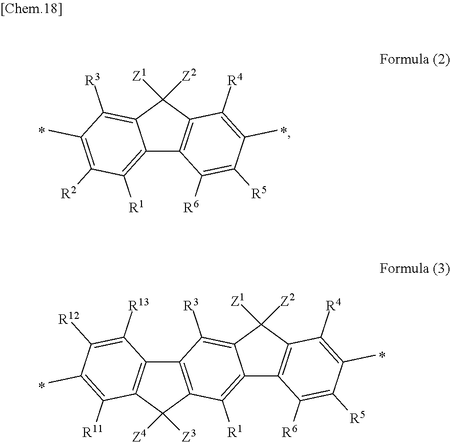

wherein R¹ to R⁶, R¹¹ to R¹⁸ and Z¹ to Z⁶ each independently represent a hydrogen atom or a substituent, R¹ and R², R⁵ and R⁶, R¹² and R¹³, and R¹⁶ and R¹⁷ may be bonded together to form a ring, and each * represents a bonding site;

BT represents a linking group represented by the following formula (4) or formula (5):

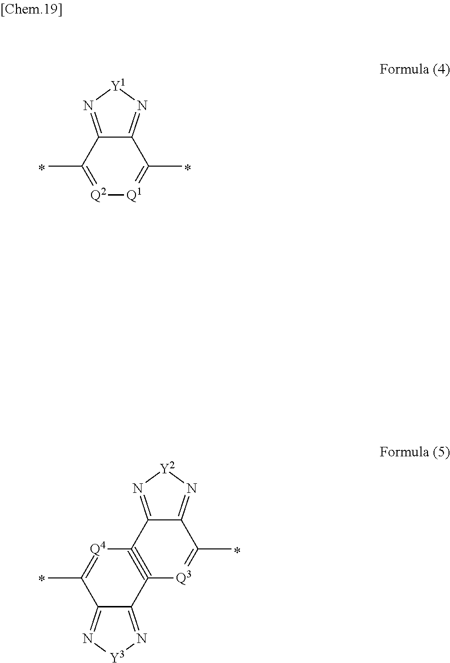

wherein Y¹ represents an oxygen atom, a sulfur atom or N-T¹, Y² represents an oxygen atom, a sulfur atom or N-T², Y³ represents an oxygen atom, a sulfur atom or N-T³, Q¹ represents a nitrogen atom or C—X¹, Q² represents a nitrogen atom or C—X², Q³ represents a nitrogen atom or C—X³, Q⁴ represents a nitrogen atom or C—X⁴, T¹ to T³ each independently represent a substituted or unsubstituted alkyl group, X¹ to X⁴ each independently represent a hydrogen atom, a substituted or unsubstituted alkyl group, a substituted or unsubstituted alkoxy group or a halogen atom, or X¹ and X² may be bonded together to form a ring, and each * represents a bonding site; n1, n2 and m each independently represent an integer of from 1 to 5; rightmost n2 may be 0;
when m is two or more, then two or more instances of BT may be the same or different, two or more instances of FL² may be the same or different, and two or more instances of n2 may be the same or different;
when n1 is two or more, then two or more instances of FL¹ may be the same or different;
when n2 is two or more, then two or more instances of FL² may be the same or different,
when n1 and m are 1, then at least one of the following three conditions are satisfied:
(1) at least one of FL¹ and FL² is a linking group represented by the formula (3),
(2) BT is a linking group represented by the formula (5), and
(3) at least one of G¹ and G² is a substituted or unsubstituted diarylamino group.

[23] Use of the compound of [1] as an emitter in an organic solid-state laser.

### Advantageous Effects of Invention

The compounds represented by the formula (1) have excellent lasing properties. An organic light-emitting device containing a compound of the formula (1) exhibits low laser oscillation threshold.

### BRIEF DESCRIPTION OF DRAWINGS

FIG. 1 shows (a) variation of the intensity as a function of the excitation intensity and variation of the FWHM as a function of the excitation intensity and (b) variation of the spectrum as a function of the excitation intensity.

FIG. 2 shows (a) the emission spectrum of the DFB laser of Example 2 and (b) the emission output intensity as a function of the pump energy density.

FIG. 3 shows (a) the emission spectrum of the DFB laser of Example 3 and (b) the emission output intensity as a function of the pump energy density.

FIG. 4 shows (a) the emission spectrum of the DFB laser of Example 4 and (b) the emission output intensity as a function of the pump energy density.

### DETAILED DESCRIPTION OF THE INVENTION

The contents of the invention will be described in detail below. The elements of the invention may be described below with reference to representative embodiments and specific examples of the invention, but the invention is not limited to the embodiments and the examples. In the description, a numerical range expressed with reference to an upper limit and/or a lower limit means a range that includes the upper limit and/or the lower limit. The room temperature means 25° C.

### Definition

The hydrogen atoms that are present in the compounds used in the invention are not particularly limited in isotope species, and for example, all the hydrogen atoms in the molecule may be ¹H, and all or a part of them may be ²H (deuterium (D)).

The alkyl group referred in the present application may be linear, branched or cyclic, and a linear or branched alkyl group is preferred. The alkyl group preferably has from 1 to 20 carbon atoms, more preferably from 1 to 12 carbon atoms (e.g., a methyl group, an ethyl group, an n-propyl group, an isopropyl group, an n-butyl group, an isobutyl group, a tert-butyl group, an n-pentyl group, an isopentyl group, an n-hexyl group, an isohexyl group, an n-heptyl group, an n-octyl group, an n-nonyl group, an n-decyl group, an n-undecyl group and an n-dodecyl group). Examples of the cyclic alkyl group include a cyclopropyl group, a cyclobutyl group, a cyclopentyl group, a cyclohexyl group, a cycloheptyl group, a bicyclo[2.1.1]hexyl group and a bicyclo[2.2.1]heptyl group. The alkyl group may be substituted. Examples of the substituent in this case include an alkoxy group, an aryl group, an aryloxy group, an acyl group, a hydroxyl group, a halogen atom, a nitro group, a diarylamino group (including a 9-carbazolyl group) and a cyano group, and preferred are an alkoxy group, an aryl group and an aryloxy group.

The aryl group referred in the present application may have a structure containing only one aromatic ring or a structure containing two or more aromatic rings condensed with each other. The aryl group preferably has from 6 to 22 ring skeleton-forming carbon atoms, more preferably from 6 to 18 ring skeleton-forming carbon atoms, further preferably from 6 to 14 ring skeleton-forming carbon atoms, and still further preferably from 6 to 10 ring skeleton-forming carbon atoms. Examples of the aryl group include a phenyl group, a 1-naphthyl group, a 2-naphthyl group, a 1-anthranyl group, a 2-anthranyl group, a 9-anthranyl group, a 1-phenanthryl group, a 2-phenanthryl group, a 3-phenanthryl group, a 4-phenanthryl group, a 9-phenanthryl group, a 1-naphthacenyl group, a 2-naphthacenyl group, a 1-pyrenyl group and a 2-pyrenyl group. The aryl group may be substituted. Examples of the substituent in this case include an alkyl group, an alkoxy group, an aryl group, an aryloxy group, an acyl group, a hydroxyl group, a halogen atom, a nitro group, a diarylamino group (including a 9-carbazolyl group) and a cyano group, and preferred are an alkyl group, an alkoxy group, an aryl group, and an aryloxy group.

The heteroaryl group referred in the present application may have a structure containing only one heteroaromatic ring or a structure containing two or more heteroaromatic rings condensed with each other. The heteroaryl group may contain at least one heteroaromatic ring and at least one aromatic ring. The heteroaryl group preferably has from 5 to 22 ring skeleton-forming atoms, more preferably from 5 to 18 ring skeleton-forming atoms, further preferably from 5 to 14 ring skeleton-forming atoms, and still further preferably from 5 to 10 ring skeleton-forming atoms. Examples of the heteroaryl group include a 2-thienyl group, a 3-thienyl group, a 2-furyl group, a 3-furyl group, a 2-pyridyl group, a 3-pyridyl group, a 4-pyridyl group, a 2-pyrazinyl group, a 2-quinolyl group, a 3-quinolyl group, a 4-quinolyl group, a 1-isoquinolyl group and a 3-isoquinolyl group. Other examples of the heteroaryl group include a benzofuryl group, a pyrrolyl group, an indolyl group, an isoindolyl group, an azaindolyl group, a benzothienyl group, a pyridyl group, a quinolinyl group, an isoquinolyl group, an imidazolyl group, a benzimidazolyl group, a pyrazolyl group, an oxazolyl group, an isoxazolyl group, a benzoxazolyl group, a thiazolyl group, a benzothiazolyl group, an isothiazolyl group, a pyridazinyl group, a pyrimidinyl group, a pyrazinyl group, a triazinyl group, a cinnolinyl group, a phthalazinyl group and a quinazolinyl group. The heteroaryl group may be substituted. Examples of the substituent in this case include an alkyl group, an alkoxy group, an aryl group, an aryloxy group, a hydroxyl group, a halogen atom, a nitro group, a diarylamino group (including a 9-carbazolyl group) and a cyano group, and preferred are an alkyl group, an alkoxy group, an aryl group, and an aryloxy group.

For the alkyl moiety of the alkoxy group and the dialkylamino group referred in the present application, reference may be made to the description for the alkyl group.

For the aryl moiety of the aryloxy group and the diarylamino group referred in the present application, reference may be made to the description for the aryl group.

The halogen atom referred in the present application is preferably a fluorine atom, a chlorine atom, a bromine atom or an iodine atom.

Compound represented by the formula (1)

The compound of the invention has a structure represented by the following formula (1):

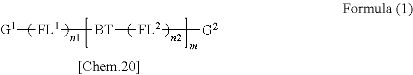

G¹ and G² in the formula (1) each independently represent a hydrogen atom or a substituent, more preferably a substituent for stability of the molecule.

The substituent for G¹ and G² is preferably a substituted or unsubstituted alkyl group, a substituted or unsubstituted aryl group, a substituted or unsubstituted heteroaryl group, a substituted or unsubstituted alkoxy group, a substituted or unsubstituted aryloxy group, a substituted or unsubstituted heteroaryloxy group, a halogen atom, or a disubstituted amino group; more preferably a substituted or unsubstituted alkyl group, a substituted or unsubstituted aryl group, or a disubstituted amino group; still more preferably a substituted or unsubstituted aryl group, or a disubstituted amino group. The disubstituted amino group is preferably a substituted or unsubstituted dialkylamino group, or a substituted or unsubstituted diarylamino group; more preferably a substituted or unsubstituted diarylamino group. Examples of the substituted or unsubstituted diarylamino group include a substituted or unsubstituted diphenylamino group such as an unsubstituted diphenylamino group, a substituted or unsubstituted dinaphthylamino group, and a substituted or unsubstituted phenylnaphthylamino group. The substituted or unsubstituted aryl group for G¹ and G² is preferably a substituted or unsubstituted diarylaminophenyl group such as a diphenylaminophenyl group and a bis(dimethoxyphenylamino)phenyl group; a substituted or unsubstituted alkoxyphenyl group such as an isopropoxyphenyl group; a substituted or unsubstituted alkylthiophenyl group such as an isopropylthiophenyl group; a substituted or unsubstituted silylphenyl group such as a triphenylsilylphenyl group; a substituted or unsubstituted alkylphenyl group such as a trifluoromethylphenyl group; a substituted or unsubstituted dibenzofuranyl group; or a substituted or unsubstituted dibenthienyl group.

G¹ and G² may be the same or different from each other. Preferably G¹ and G² are the same. More preferably, G¹ and G² are a substituent.

BT in the formula (1) represents a linking group represented by the following formula (4) or formula (5):

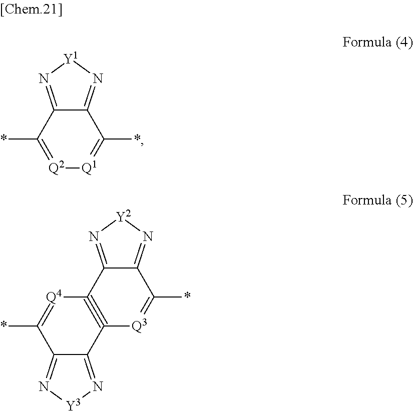

In the formulae (4) and (5), Y¹ represents an oxygen atom, a sulfur atom or N-T¹, Y² represents an oxygen atom, a sulfur atom or N-T², Y³ represents an oxygen atom, a sulfur atom or N-T³. Y² and Y³ may be the same or different from each other. Preferably Y² and Y³ are the same. Y¹, Y² and Y³ may be an oxygen atom or a sulfur atom. Y¹, Y² and Y³ may be N-T¹, N-T² and N-T³, respectively. T¹ to T³ each independently represent a substituted or unsubstituted alkyl group. The substituent on the alkyl group may be, for example, a substituted or unsubstituted aryl group, a substituted or unsubstituted heteroaryl group, a substituted or unsubstituted alkoxy group, a substituted or unsubstituted aryloxy group, a substituted or unsubstituted heteroaryloxy group, a halogen atom. T¹ to T³ may be an unsubstituted alkyl group. In some embodiments, T¹ to T³ are an alkyl group having 7 to 12 carbon atoms. T² and T 3 may be the same or different from each other. Preferably T² and T³ are the same.

In the formulae (4) and (5), Q¹ represents a nitrogen atom or C—X¹, Q² represents a nitrogen atom or C—X², Q³ represents a nitrogen atom or C—X¹, Q⁴ represents a nitrogen atom or C—X⁴. Q¹ and Q² may be the same or different from each other. Preferably Q¹ and Q² are the same. Q³ and Q⁴ may be the same or different from each other. Preferably Q³ and Q⁴ are the same. X¹ to X⁴ each independently represent a hydrogen atom, a substituted or unsubstituted alkyl group, a substituted or unsubstituted alkoxy group or a halogen atom. In some embodiments, X¹ to X⁴ are a hydrogen atom. In some embodiments, X¹ to X⁴ are a substituted or unsubstituted alkoxy group having 1 to 12 carbon atoms, for example 7 to 12 carbon atoms. The substituent on the alkyl group and the alkoxy group may be, for example, a substituted or unsubstituted aryl group, a substituted or unsubstituted heteroaryl group, a substituted or unsubstituted alkoxy group, a substituted or unsubstituted aryloxy group, a substituted or unsubstituted heteroaryloxy group, a halogen atom. In some embodiments, X¹ to X⁴ are a halogen atom such as a fluorine atom. X¹ and X² may be bonded together to form a ring. The formed ring may have from 4 to 10 ring skeleton-forming atoms, more preferably from 5 to 8 ring skeleton-forming atoms, further preferably from 5 to 7 ring skeleton-forming atoms. The formed ring may be an aromatic ring or an aliphatic ring. Examples of the ring include a cyclopentane ring, a cyclohexane ring and a cycloheptane ring, a benzene ring, a naphthalene ring and a pyridine ring.

BT in the formula (1) may be a linking group represented by the following formula (4a):

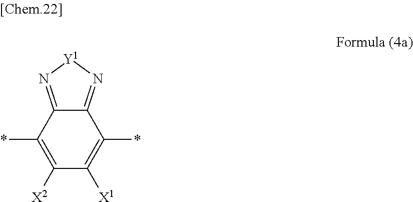

In the formula (4a), Y¹ represents an oxygen atom, a sulfur atom or N-T¹, T¹ represents a substituted or unsubstituted alkyl group, X¹ and X² each independently represent a hydrogen atom, a substituted or unsubstituted alkyl group, a substituted or unsubstituted alkoxy group or a halogen atom, or X¹ and X² may be bonded together to form a ring, and each * represents a bonding site.

BT in the formula (1) may be a linking group represented by the following formula (4b):

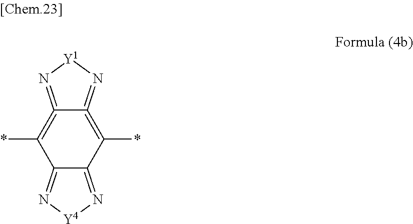

In the formula (4b), Y¹ represents an oxygen atom, a sulfur atom or N-T¹, Y⁴ represents an oxygen atom, a sulfur atom or N-T⁴, T¹ and T⁴ each independently represent a substituted or unsubstituted alkyl group, and each * represents a bonding site.

BT in the formula (1) may be a linking group represented by the following formula (4c):

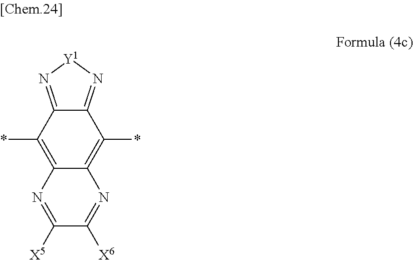

In the formula (4c), Y¹ represents an oxygen atom, a sulfur atom or N-T¹, T¹ represent a substituted or unsubstituted alkyl group, X⁵ and X⁶ each independently represent a hydrogen atom a substituted or unsubstituted alkyl group, X⁵ and X⁶ can be bonded together to form a ring, and each * represents a bonding site. X⁵ and X⁶ may be the same or different from each other. Preferably X⁵ and X⁶ are the same. In some embodiments, X⁵ and X⁶ are a substituted or unsubstituted alkyl group, more preferably an unsubstituted alky group. In some embodiments, X⁵ and X⁶ are a substituted or unsubstituted alky group having 1 to 12 carbon atoms. For the substituent on the alkyl group in X⁵ and X⁶, reference may be made to the description for the substituent on the alkyl in X¹ to X⁴. For the ring formed by bonding X⁵ and X⁶ together, reference may be made to the description for the ring formed by bonding X¹ and X² together.

BT in the formula (1) may be a linking group represented by the following formula (5a):

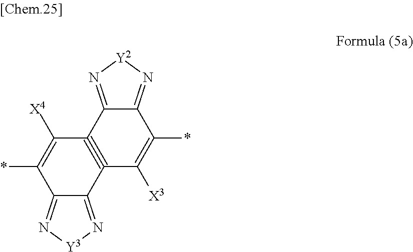

In the formula (5a), Y² represents an oxygen atom, a sulfur atom or N-T², Y³ represents an oxygen atom, a sulfur atom or N-T³, T² and T³ each independently represent a substituted or unsubstituted alkyl group, X³ and X⁴ each independently represent a hydrogen atom, a substituted or unsubstituted alkyl group, a substituted or unsubstituted alkoxy group or a halogen atom, and each * represents a bonding site.

For Y¹, Y², Y³, Y⁴, X¹, X², X³, X⁴, T¹, T² and T³ in the formulae (4a), (4b) and (5a), reference may be made to the description for Y¹, Y², Y³, Y⁴, X¹, X², X³, X⁴, T¹, T² and T³ in the formulae (4) and (5).

BT in the formula (1) is preferably selected from the group consisting of the following BT1 to BT12:

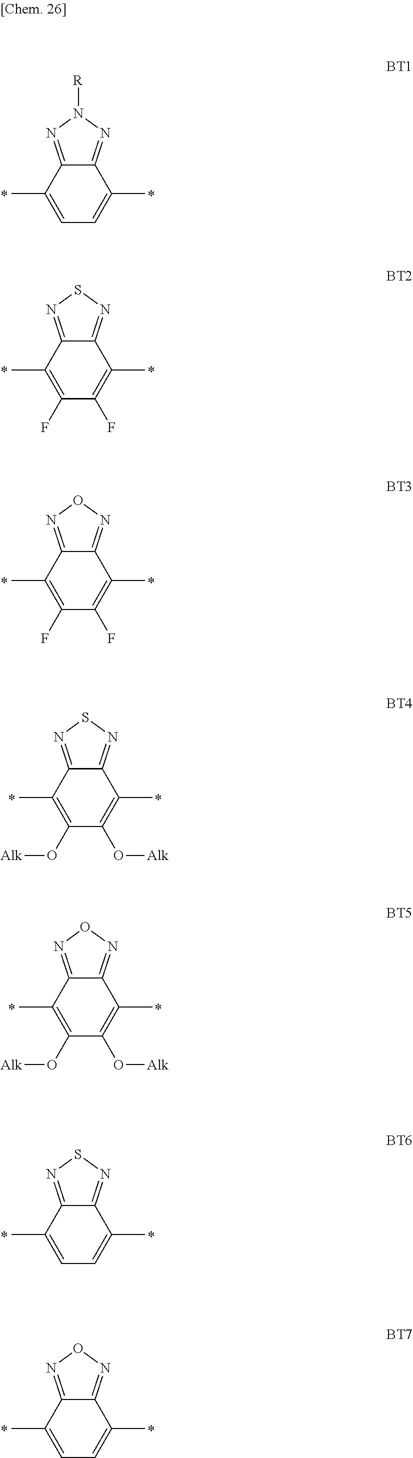

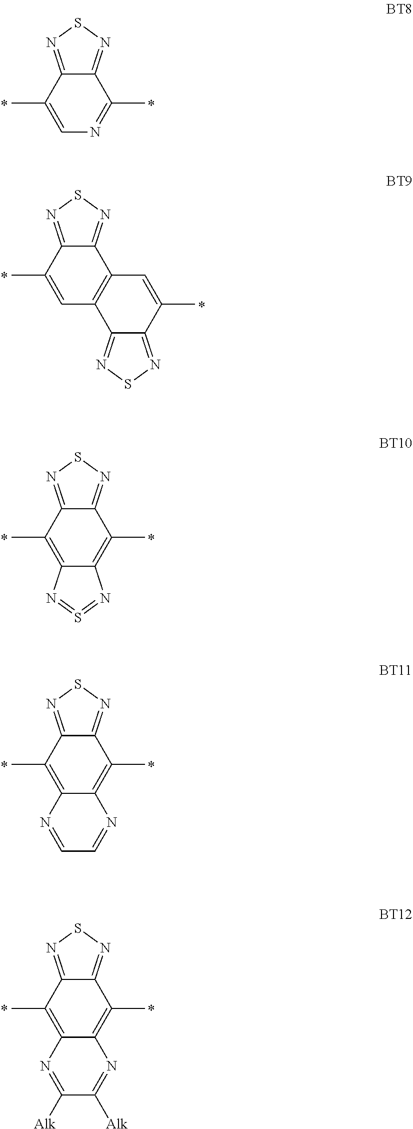

In the above formulae, R and Alk each independently represent a substituted or unsubstituted alkyl group and each * represents a bonding site. In some embodiments, BT is selected from the group consisting of BT2, BT3, BT4 and BT5. In some embodiments, BT is selected from the group consisting of BT2, BT4, BT6, BT8, BT9 and BT10. In some embodiments, BT is selected from the group consisting of BT3, BT5 and BT7. In some embodiments, BT is selected from the group consisting of BT9 and BT10. BT can be determined depending on the desired emission wavelength.

FL¹ and FL² in the formula (1) each independently represent a linking group represented by the following formula (2) or formula (3):

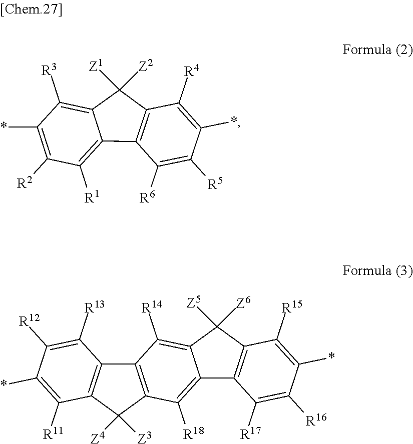

In the formulae (2) and (3), R¹ to R⁶, R¹¹ to R¹⁸ and Z¹ to Z⁶ each independently represent a hydrogen atom or a substituent, R¹ and R², R⁵ and R⁶, R¹² and R¹³, and R¹⁶ and R¹⁷ may be bonded together to form a ring, and each * represents a bonding site. FL¹ and FL² are essential for low threshold lasing properties.

In some embodiments, R¹ to R⁶, R¹¹ to R¹⁸ are a hydrogen atom. In some embodiments, Z¹ to Z⁶ are a substituted or unsubstituted alkyl group, more preferably an unsubstituted alky group. The substituent on the alkyl group may be, for example, a substituted or unsubstituted aryl group, a substituted or unsubstituted heteroaryl group, a substituted or unsubstituted alkoxy group, a substituted or unsubstituted aryloxy group, a substituted or unsubstituted heteroaryloxy group, a halogen atom. In some embodiments, Z¹ to Z⁶ are an alkyl group having from 1 to 12 carbon atoms. In some embodiments, Z¹ to Z⁶ are an alkyl group having from 7 to 12 carbon atoms. Z¹ and Z² may be the same or different from each other. Preferably Z¹ and Z² are the same. Z³ and Z⁴ may be the same or different from each other. Preferably Z³ and Z⁴ are the same. Z⁵ and Z⁶ may be the same or different from each other. Preferably Z⁵ and Z⁶ are the same. More preferably Z³, Z⁴, Z⁵ and Z⁶ are the same.

n1, n2 and m in the formula (1) each independently represent an integer of from 1 to 5. In some embodiments, n1, n2 are an integer of from 1 to 3 and m is an integer of from 1 to 4. In some embodiments, at least one of n1 and n2 is 2. In some embodiments, at least one of n1 and n2 is 2, and all of the other n1 and n2 are 1. In some embodiments, at least one of n1 and n2 is 3. In some embodiments, at least one of n1 and n2 is 3, and all of the other n1 and n2 are 1. In some embodiments, n1 and n2 are 1. In some embodiments, the sum of n1 and n2 is from 2 to 8. In some embodiments, the sum of n1 and n2 is from 3 to 8. In some embodiments, the sum of n1 and n2 is from 4 to 8. In some embodiments, the sum of n1 and n2 is from 5 to 8. In some embodiments, m is an integer of from 1 to 5. In some embodiments, m is an integer of from 2 to 5. In some embodiments, m is an integer of from 3 to 5. In some embodiments, m is an integer of from 4 or 5.

Rightmost n2 may be 0. When m is 1, then n2 may be 0 or an integer of from 1 to 5. In some embodiments, when m is 1, then n2 is an integer of from 1 to 5. In some embodiments, when m is 1, then G¹ and G² are a substituent. In some embodiments, when m is 1, then at least one of n1 and n2 is an integer of 2 to 6. In some embodiments, when m is 1, then n1 is 1 and n2 is 0 (see Compounds 48-50, 53 and 54 below). In some embodiments, when m is 1, then n1 is 1 and n2 is an integer of from 1 to 5. When m is an integer of from 2 to 5, then rightmost n2 may be 0 or an integer of from 1 to 5. In some embodiments, m is an integer of from 2 to 5 and rightmost n2 is 0. In some embodiments, m is an integer of from 2 to 5 and rightmost n2 is 1. In some embodiments, m is an integer of from 2 to 5, rightmost n2 is 1 and the other n2 is/are also 1.

When m is an integer of from 2 to 5 and rightmost n2 is 0, then n1 may be 0. In the case that m is 2, rightmost n2 is 0, the other n2 is 1 and n1 is 0, then the compound is represented by G¹-BT-FL²-BT-G² (see Compound 57 below).

When m is two or more, then two or more instances of BT may be the same or different, preferably the same. When m is two or more, then two or more instances of FL² may be the same or different, preferably the same. When m is two or more, then two or more instances of n2 may be the same or different, preferably the same. When n1 is two or more, then two or more instances of FL¹ may be the same or different, preferably the same. When n2 is two or more, then two or more instances of FL² may be the same or different, preferably the same.

In some embodiments, the compound of the formula (1) is represented by the following formula (1a):

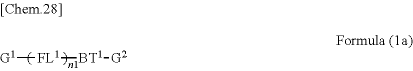

In the formula (1a), G¹ and G² each independently represent a hydrogen atom or a substituent, FL¹ represents a linking group represented by the formula (2) or formula (3), BT¹ represents a linking group represented by the formula (4) or formula (5), and n1 is an integer of from 1 to 5. In some preferred embodiments, G¹ and G² are the same.

In some embodiments, the compound of the formula (1) is represented by the following formula (1b):

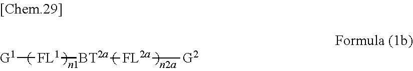

In the formula (1b), G¹ and G² each independently represent a hydrogen atom or a substituent, FL¹ and FL^{2a} each independently represent a linking group represented by the formula (2) or formula (3), BT^{2a} represents a linking group represented by the formula (4) or formula (5), and n1 and n2a are each independently an integer of from 1 to 5. In some embodiments, n1 is 1. In some embodiments, n1 is 2. In some embodiments, n2a is 1. In some embodiments, n2a is 2. In some embodiments, n1 and n2a are 1. In some embodiments, n1 and n2a are 2. In some preferred embodiments, FL¹ and FL^{2a} are the same. In some preferred embodiments, G¹ and G² are the same. In some preferred embodiments, the compound is symmetric.

In some embodiments, the compound of the formula (1) is represented by the following formula (1c):

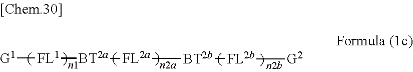

In the formula (1c), G¹ and G² each independently represent a hydrogen atom or a substituent, FL¹, FL^{2a} and FL^{2b} each independently represent a linking group represented by the formula (2) or formula (3), BT^{2a} and BT^{2b} each independently represent a linking group represented by the formula (4) or formula (5), n1 and n2a are each independently an integer of from 1 to 5, and n2b is an integer of from 0 to 5. In some embodiments, n1 is 1. In some embodiments, n1 is 2. In some embodiments, n2a is 1. In some embodiments, n2a is 2. In some embodiments, n2a is 3. In some embodiments, n2b is 0. In some embodiments, n2b is 1. In some embodiments, n2b is 2. In some preferred embodiments, n1, n2a and n2b are 1. In some preferred embodiments, n1 and n2a are 1, and n2b is 0. In some preferred embodiments, n1 and n2b are 1, and n2a is 3. In some preferred embodiments, n1 and n2b are 2, and n2a is 5. In some preferred embodiments, FL¹, FL^{2a} and FL^{2b} are the same. In some preferred embodiments, BT^{2a} and BT^{2b} are the same. In some preferred embodiments, G¹ and G² are the same. In some preferred embodiments, the compound is symmetric.

In some embodiments, the compound of the formula (1) is represented by the following formula (1d): 7

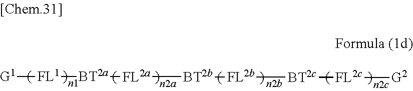

In the formula (1d), G¹ and G² each independently represent a hydrogen atom or a substituent, FL¹, FL^{2a}, FL^{2b} and FL^{2c} each independently represent a linking group represented by the formula (2) or formula (3), BT^{2a}, BT^{2b} and BT^{2c} each independently represent a linking group represented by the formula (4) or formula (5), n1, n2a and n2b are each independently an integer of from 1 to 5, and n2c is an integer of from 0 to 5. In some embodiments, n1 is 1. In some embodiments, n1 is 2. In some embodiments, n2a is 1. In some embodiments, n2a is 2. In some embodiments, n2b is 0. In some embodiments, n2b is 1. In some embodiments, n2c is 0. In some embodiments, n2c is 1. In some embodiments, n2c is 2. In some preferred embodiments, n1, n2a, n2b and n2c are 1. In some preferred embodiments, n1, n2a and n2b are 1, and n2c is 0. In some preferred embodiments, FL¹, FL^{2d}, FL^{2b} and FL^{2c} are the same. In some preferred embodiments, BT^{2a}, BT^{2b} and BT^{2c} are the same. In some preferred embodiments, G¹ and G² are the same. In some preferred embodiments, the compound is symmetric.

In some embodiments, the compound of the formula (1) is represented by the following formula (1e):

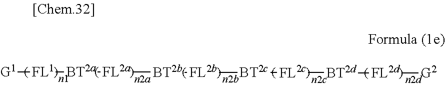

In the formula (1e), G¹ and G² each independently represent a hydrogen atom or a substituent, FL¹, FL^{2a}, FL^{2b}, FL^{2c} and FL^{2d} each independently represent a linking group represented by the formula (2) or formula (3), BT^{2a}, BT^{2b}, BT^{2c} and BT^{2d} each independently represent a linking group represented by the formula (4) or formula (5), n1, n2a, n2b and n2c are each independently an integer of from 1 to 5, and n2d is an integer of from 0 to 5. In some embodiments, n1 is 1. In some embodiments, n1 is 2. In some embodiments, n2a is 1. In some embodiments, n2a is 2. In some embodiments, n2b is 1. In some embodiments, n2b is 2. In some embodiments, n2c is 1. In some embodiments, n2c is 2. In some embodiments, n2d is 0. In some embodiments, n2d is 1. In some embodiments, n2d is 2. In some preferred embodiments, n1, n2a, n2b, n2c and n2d are 1. In some preferred embodiments, n1, n2a, n2b and n2c are 1, and n2d is 0. In some preferred embodiments, n1, n2a, n2c and n2d are 1, and n2b is 3. In some preferred embodiments, FL¹, FL^{2d}, FL^{2b}, FL^{2c} and FL^{2d} are the same. In some preferred embodiments, BT^{2d}, BT^{2b}, BT^{2c} and BT^{2d} are the same. In some preferred embodiments, G¹ and G² are the same. In some preferred embodiments, the compound is symmetric.

In some embodiments, the compound of the formula (1) is represented by the following formula (1f):

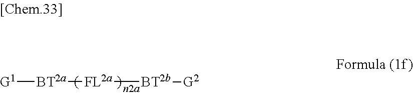

In the formula (1f), G¹ and G² each independently represent a hydrogen atom or a substituent, FL^{2d} represents a linking group represented by the formula (2) or formula (3), BT^{2d} and BT^{2b} each independently represent a linking group represented by the formula (4) or formula (5), and n2a is an integer of from 1 to 5. In some embodiments, n2a is 1. In some embodiments, n2a is 2. In some preferred embodiments, BT^{2a} and BT^{2b} are the same. In some preferred embodiments, G¹ and G² are the same.

In some embodiments, the compound of the formula (1) is represented by the following formula (1g):

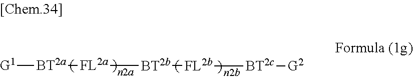

In the formula (1g), G¹ and G² each independently represent a hydrogen atom or a substituent, FL^{2a} and FL^{2b} each independently represent a linking group represented by the formula (2) or formula (3), BT^{2a}, BT^{2b} and BT^{2c} each independently represent a linking group represented by the formula (4) or formula (5), and n2a and n2b are each independently an integer of from 1 to 5. In some embodiments, n2a is 1. In some embodiments, n2a is 2. In some embodiments, n2b is 1. In some embodiments, n2b is 2. In some preferred embodiments, FL^{2a} and FL^{2b} are the same. In some preferred embodiments, BT^{2a}, BT^{2b} and BT^{2c} are the same. In some preferred embodiments, G¹ and G² are the same.

In some embodiments, the compound of the formula (1) is represented by the following formula (1h):

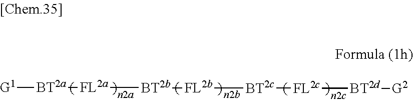

In the formula (1h), G¹ and G² each independently represent a hydrogen atom or a substituent, FL^{2a}, FL^{2b} and FL^{2c} each independently represent a linking group represented by the formula (2) or formula (3), BT^{2a}, BT^{2b}, BT^{2c} and BT^{2d} each independently represent a linking group represented by the formula (4) or formula (5), and n2a, n2b and n2c are each independently an integer of from 1 to 5. In some embodiments, n2a is 1. In some embodiments, n2a is 2. In some embodiments, n2b is 1. In some embodiments, n2b is 2. In some embodiments, n2c is 1. In some embodiments, n2c is 2. In some preferred embodiments, FL^{2a}, FL^{2b} and FL^{2c} are the same. In some preferred embodiments, BT^{2a}, BT^{2b}, BT^{2c} and BT^{2d} are the same. In some preferred embodiments, G¹ and G² are the same. For G¹, G², BT¹, BT^{2a}, BT^{2b}, BT^{2c}, BT^{2d}, FL¹, FL^{2a}, FL^{2b}, FL^{2c}, FL^{2d}, n1, n2a, n2b, n2c and n2d in the formulae (1a) to (1h), reference may be made to the corresponding description in the formulae (1) to (5).

Specific examples of the compounds represented by the formula (1) shown below. However, the compounds represented by the formula (1) capable of being used in the invention are not limited to the specific examples.

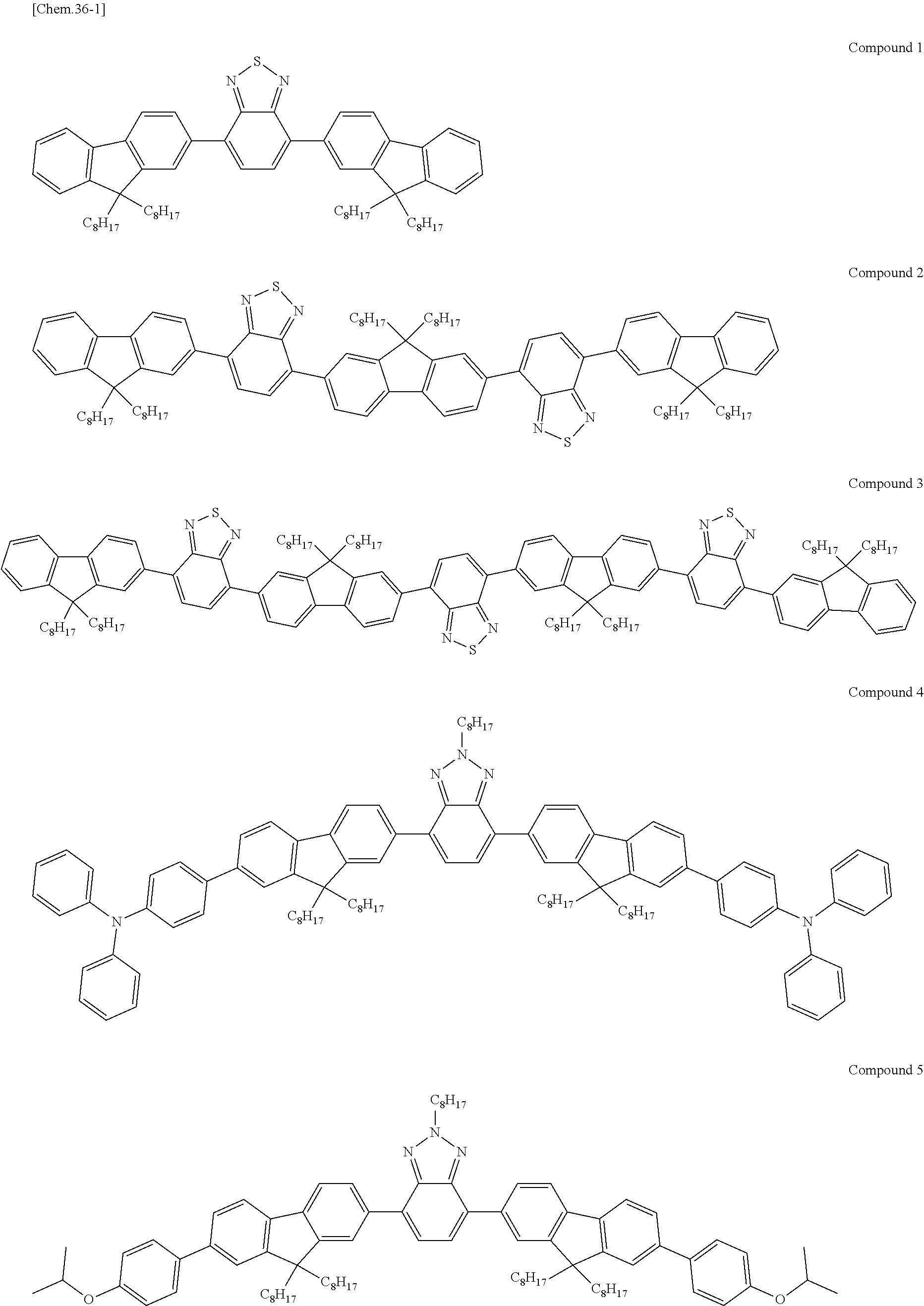

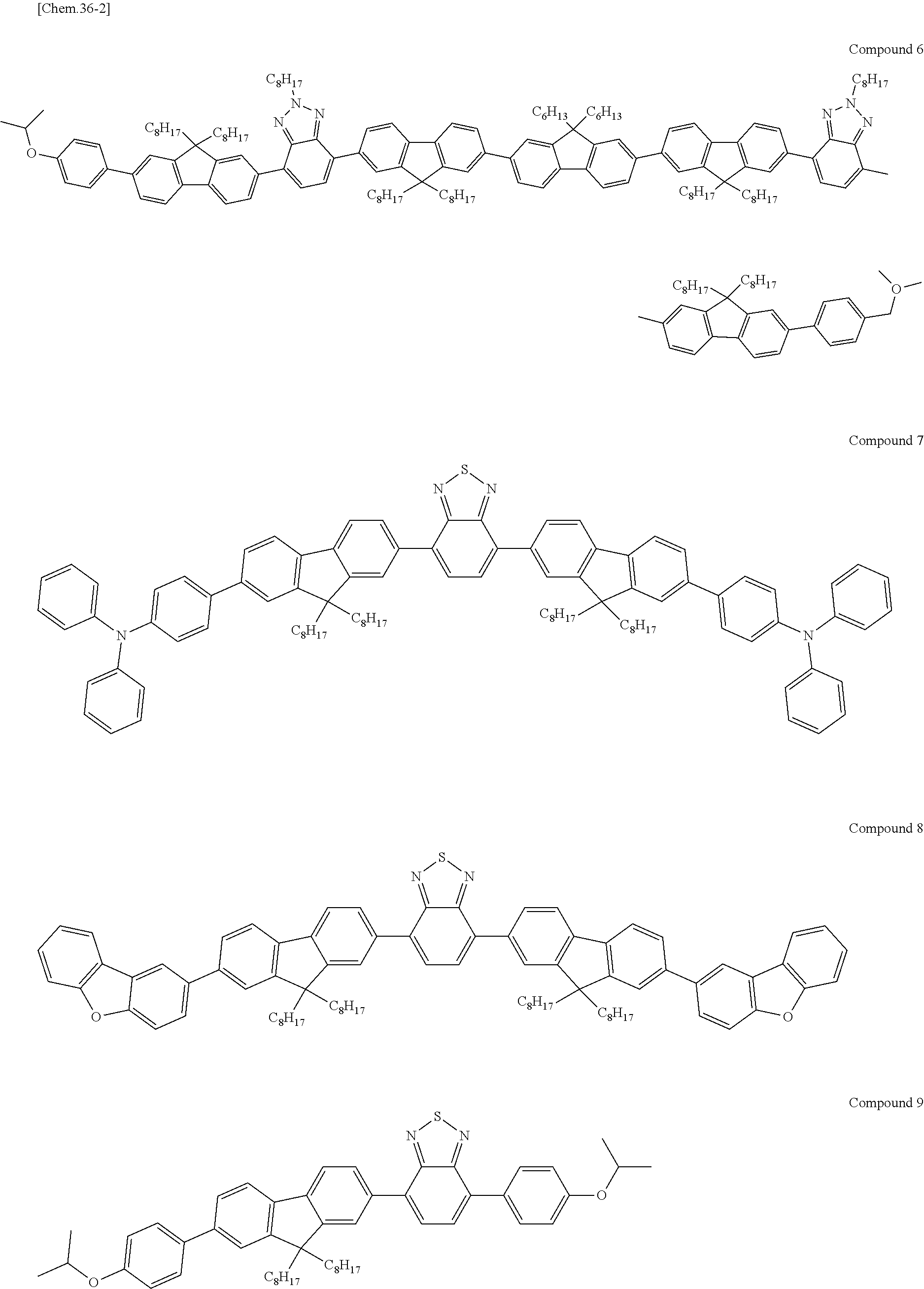

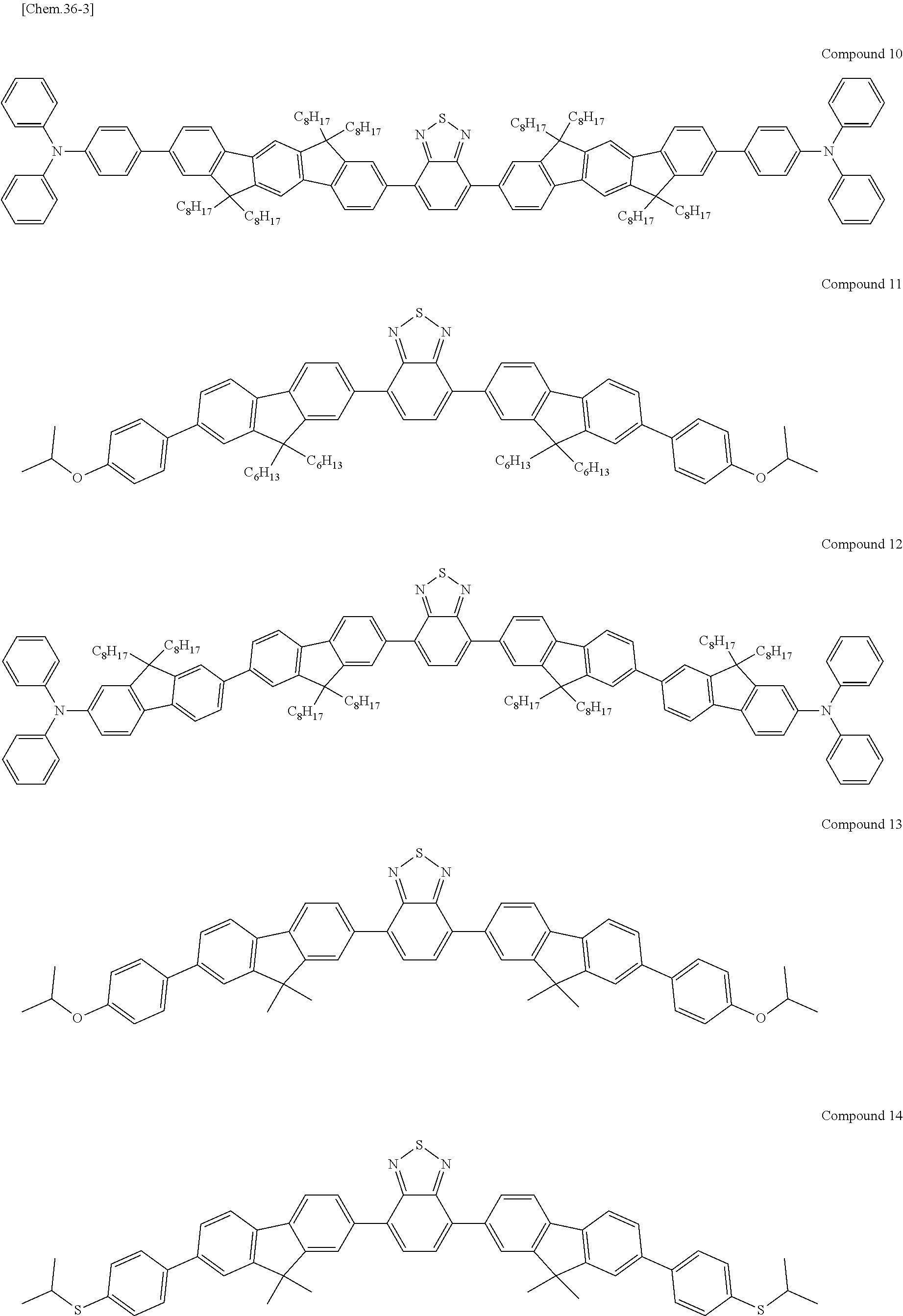

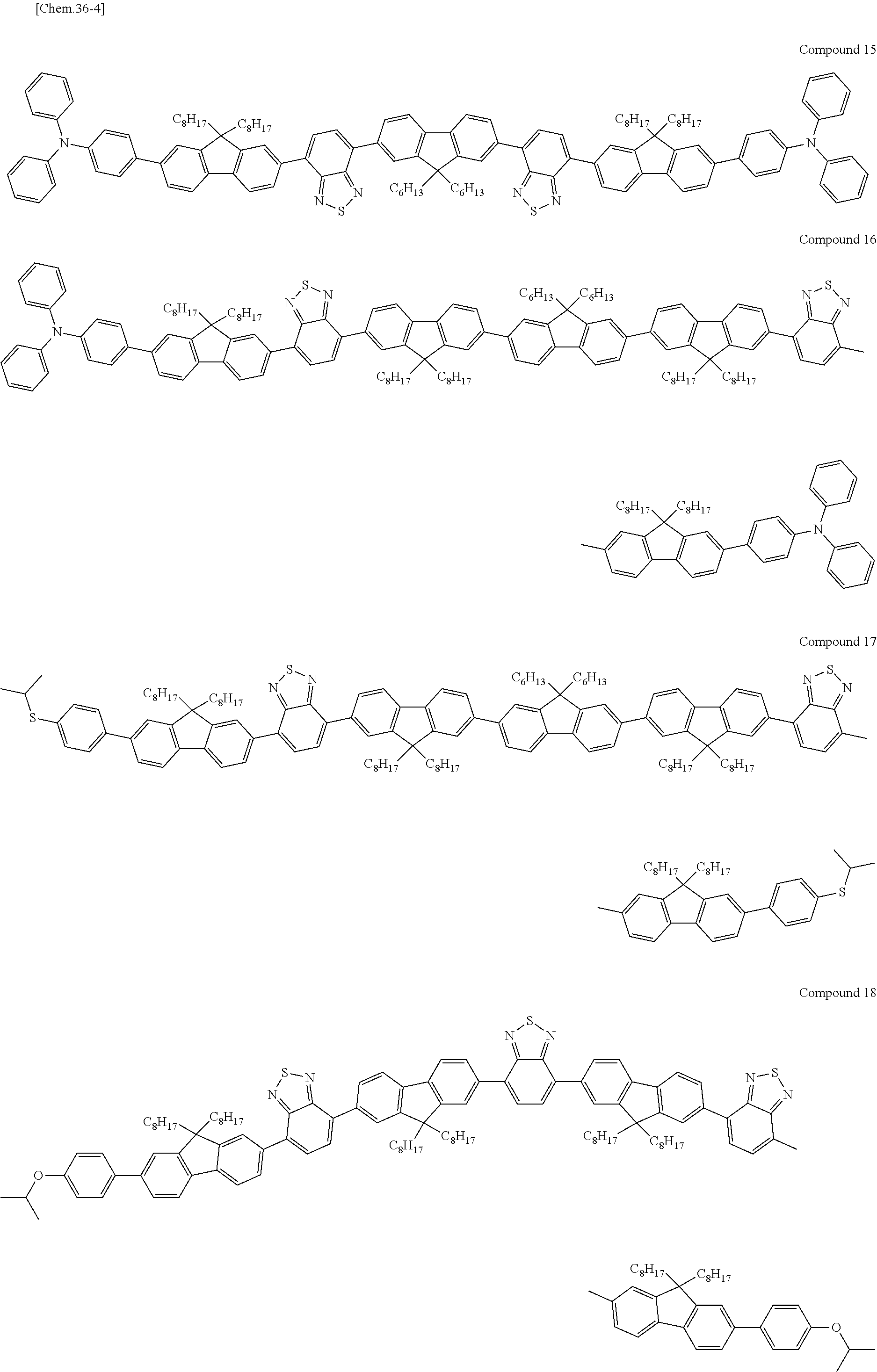

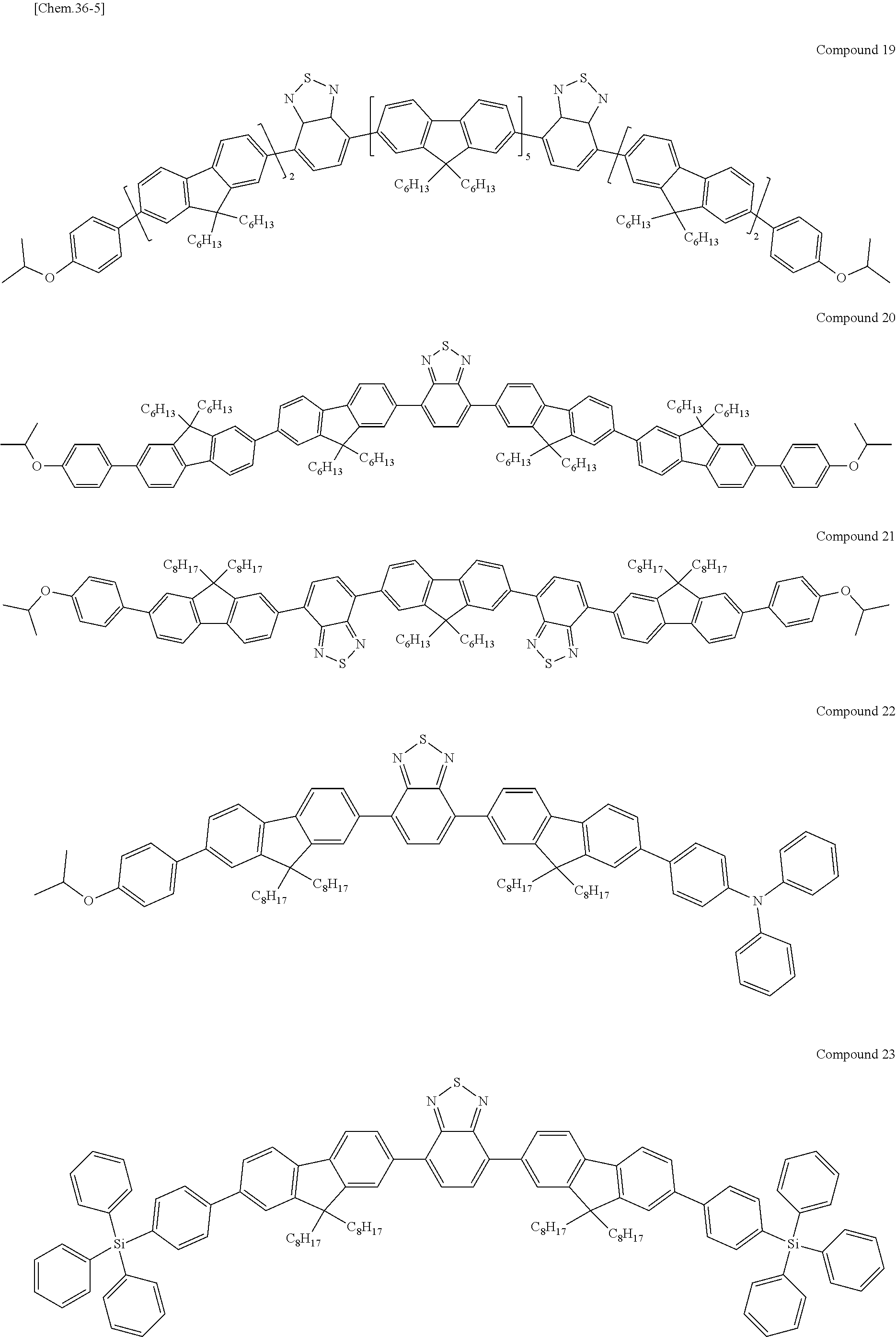

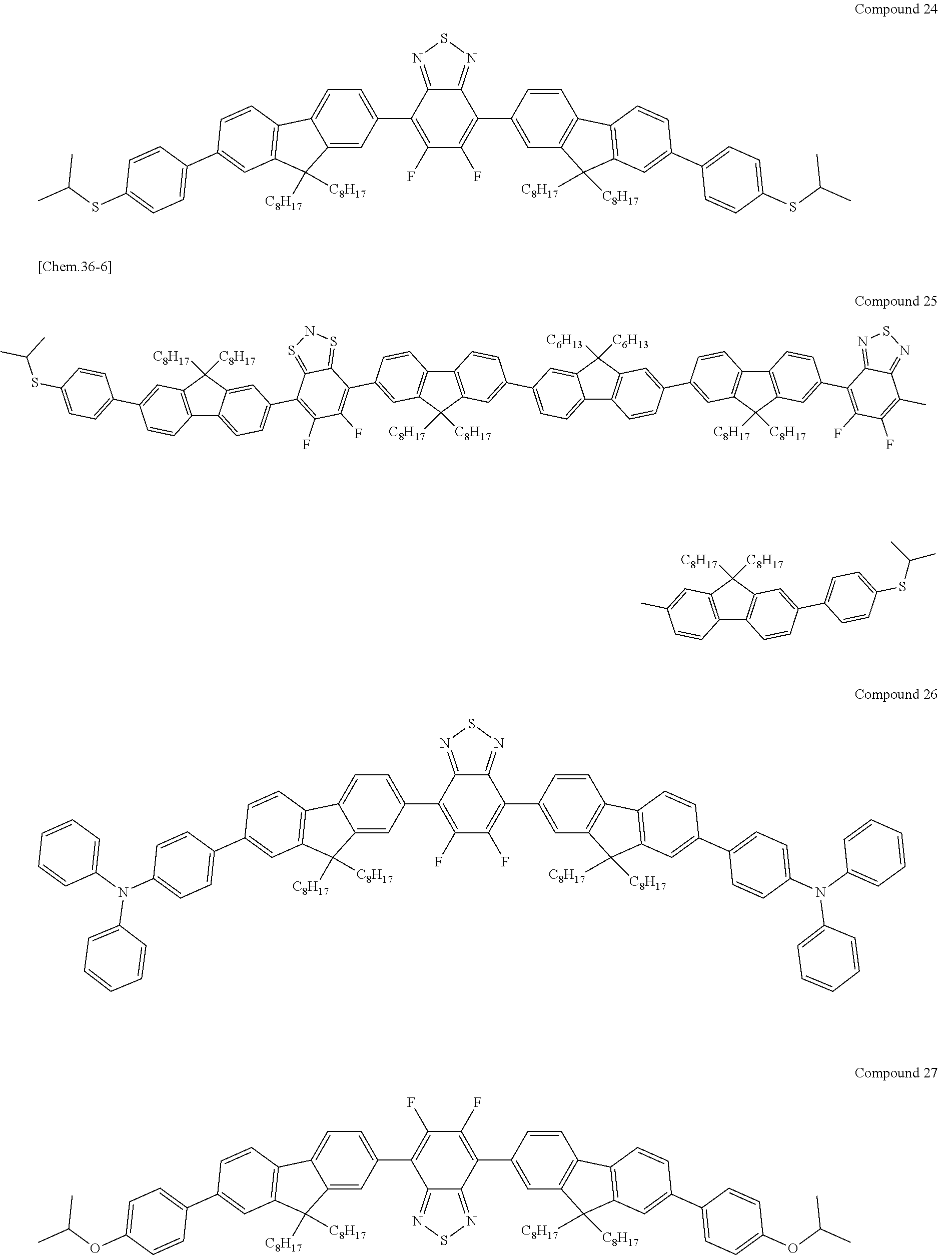

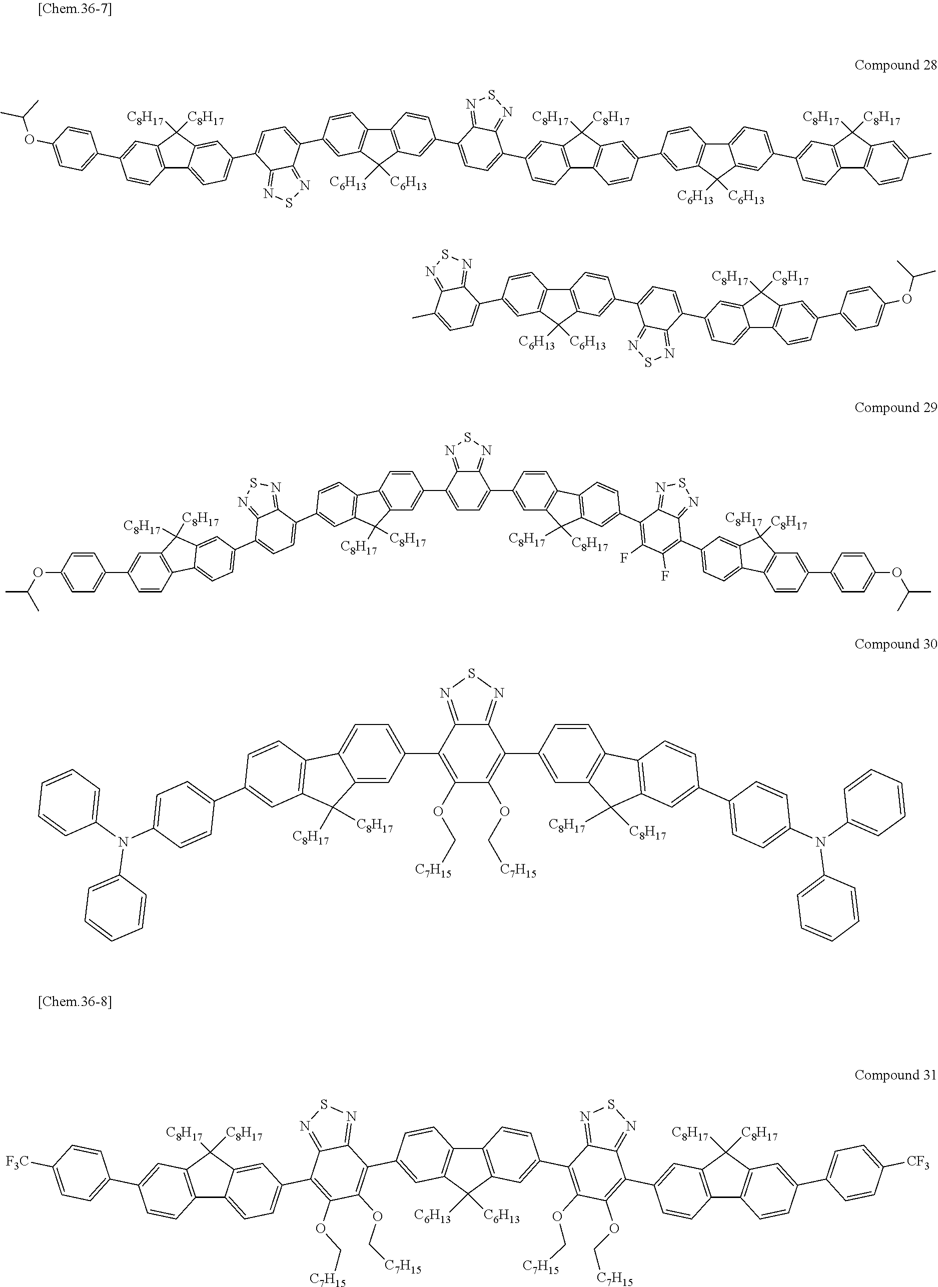

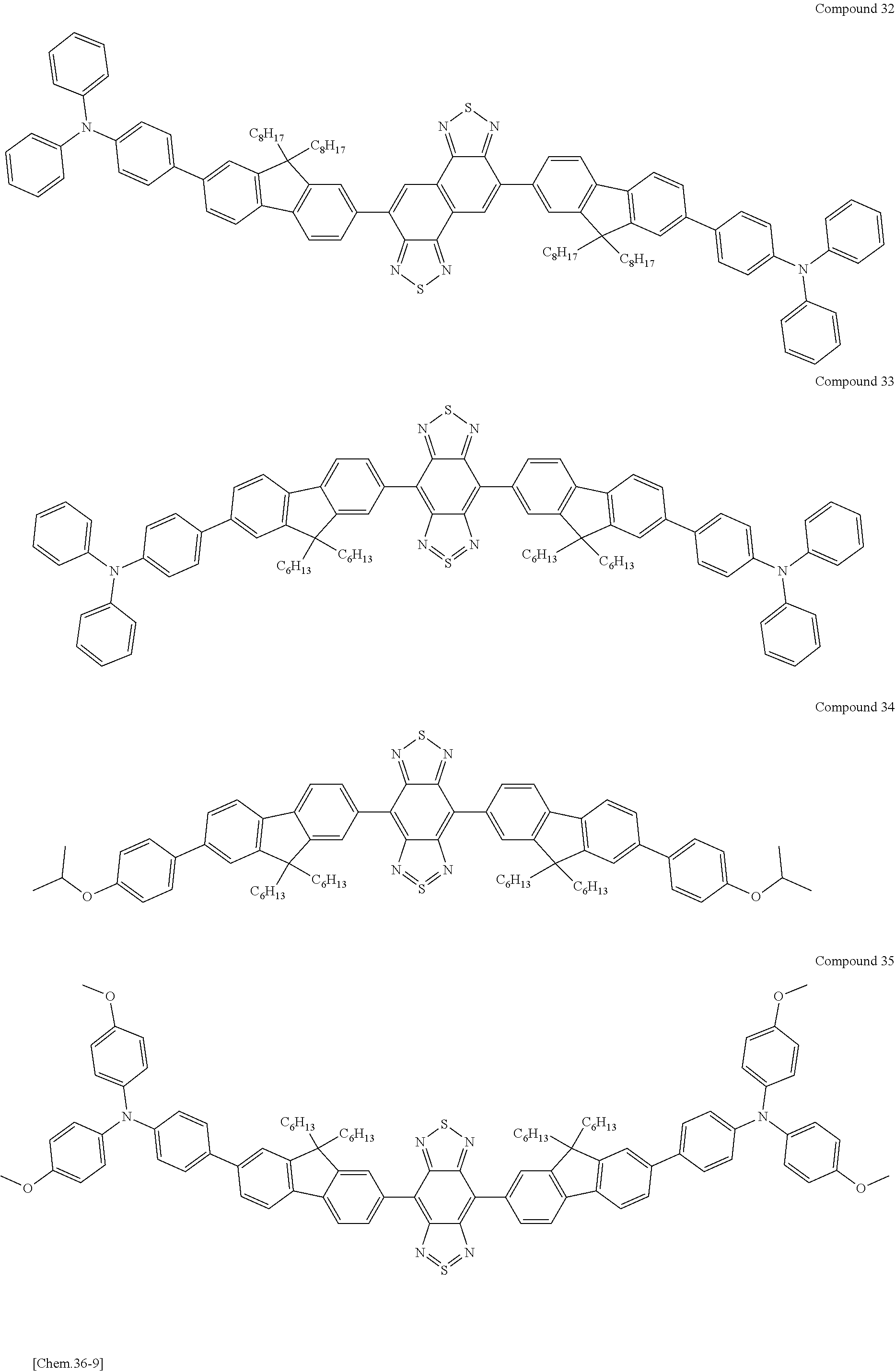

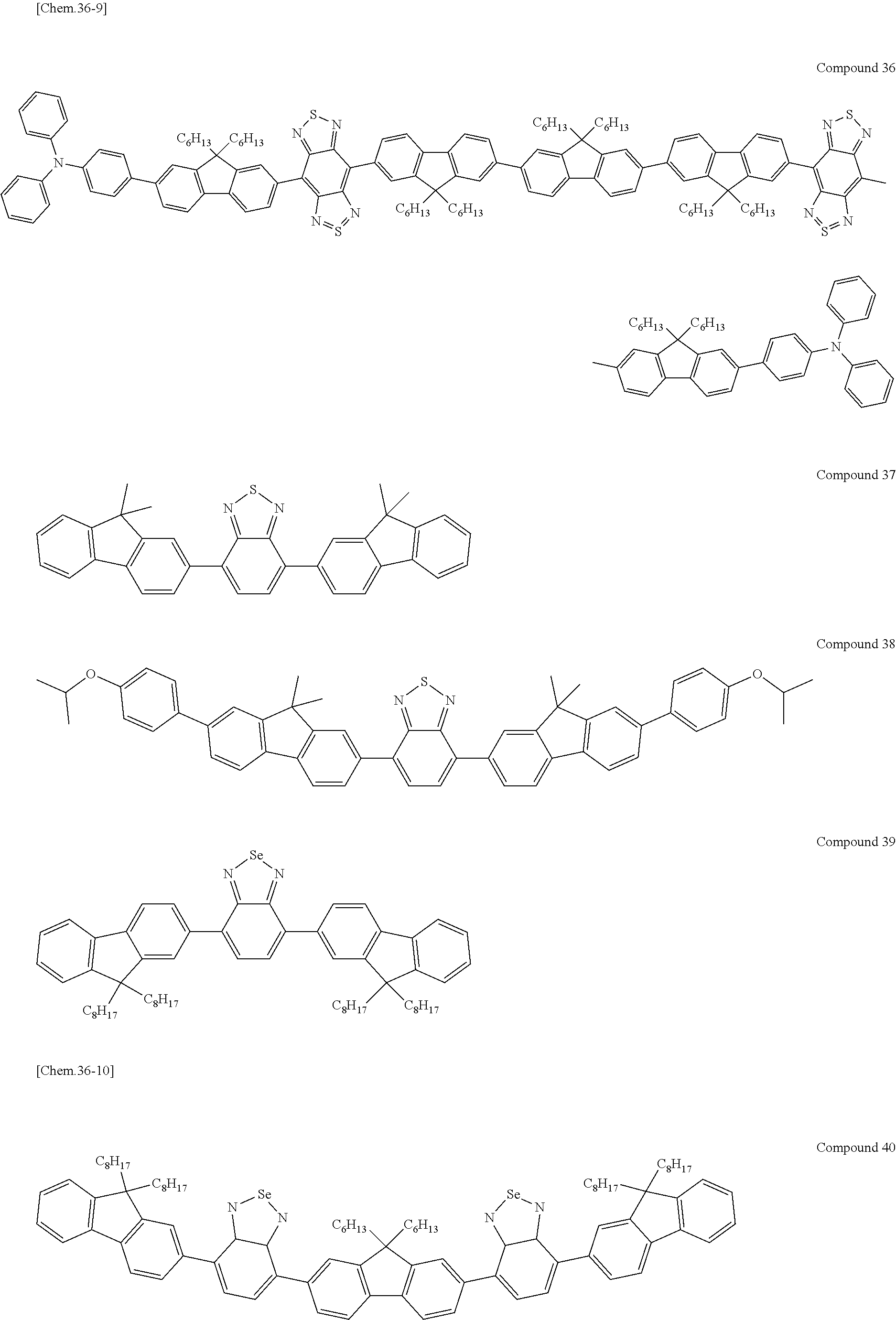

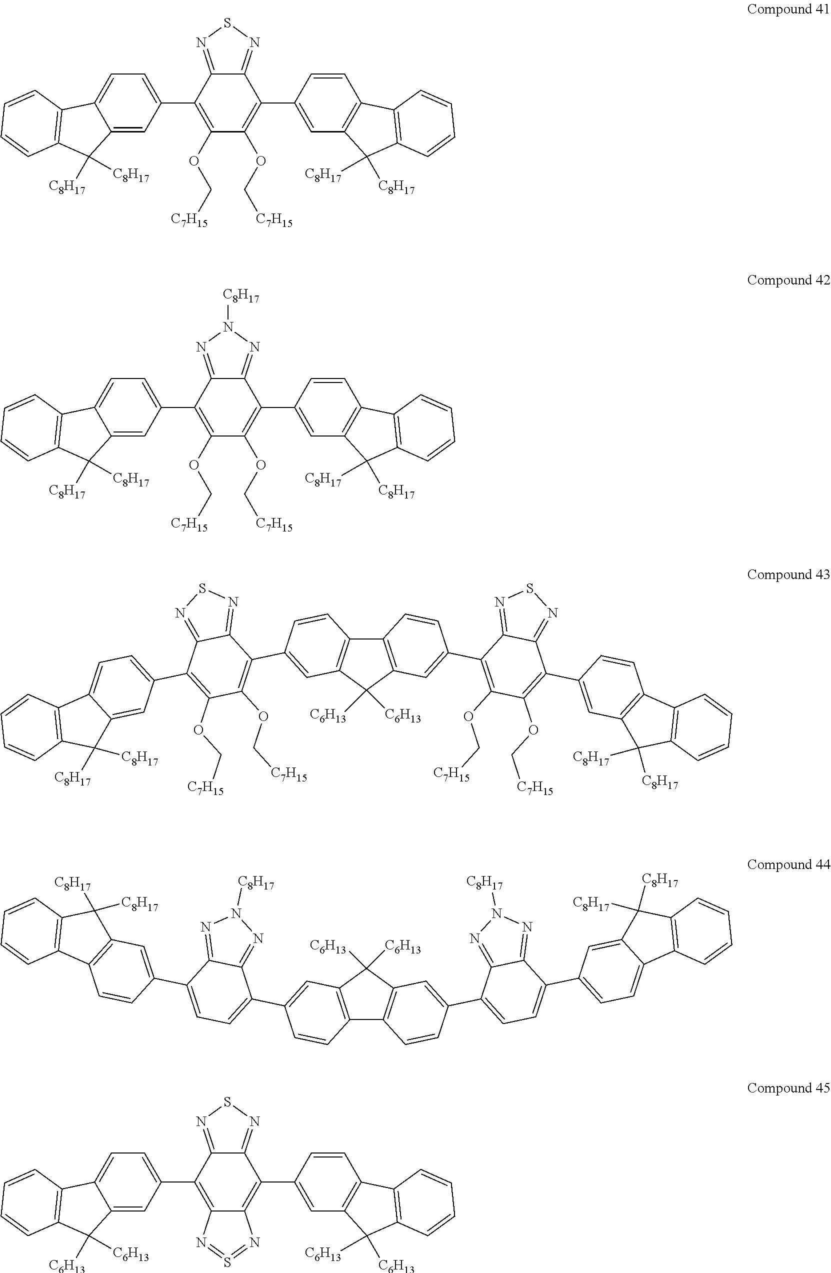

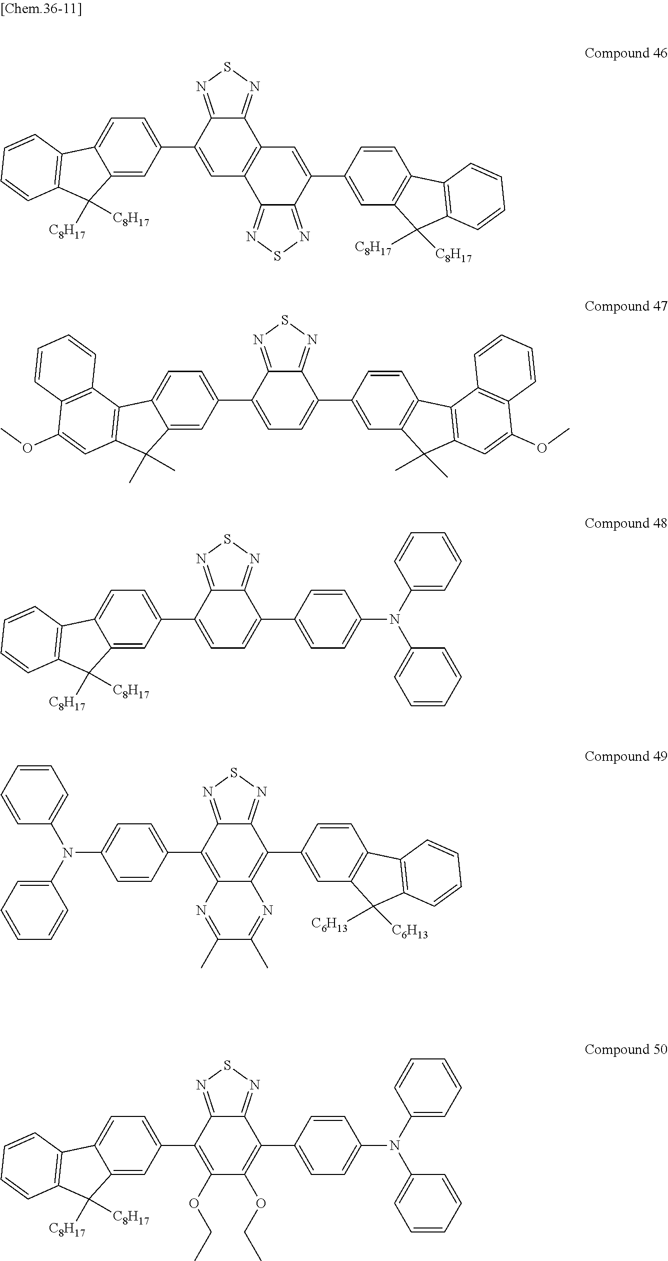

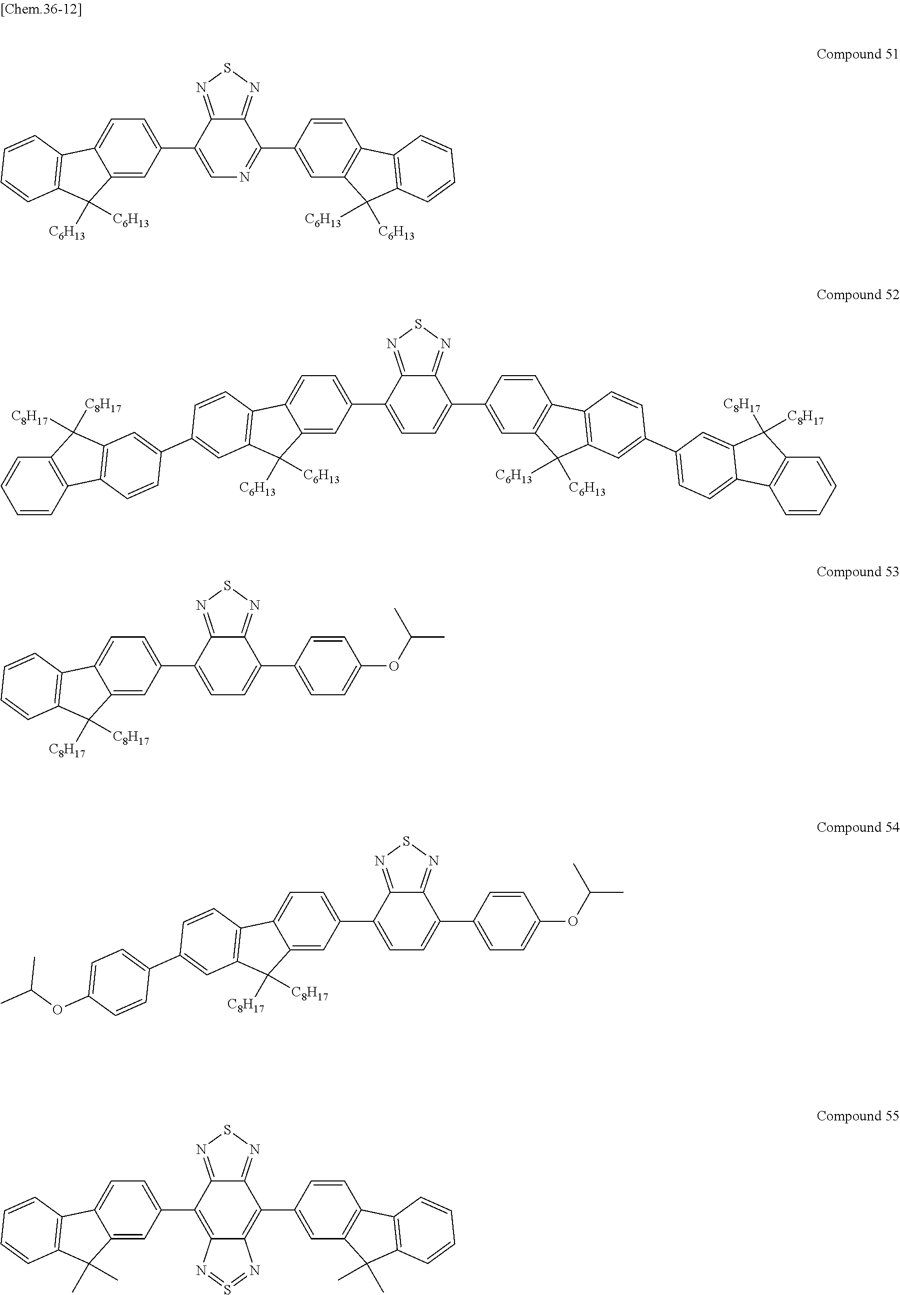

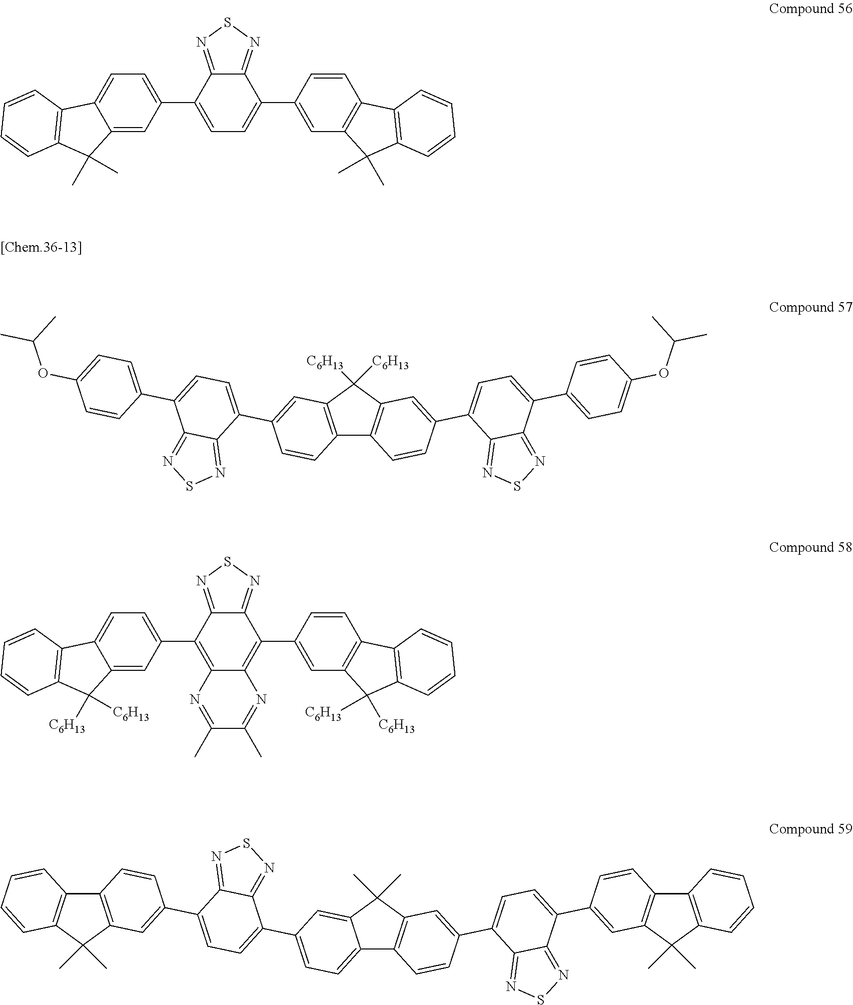

The molecular weight of the compound represented by the formula (1) is preferably 1,500 or less, more preferably 1,200 or less, further preferably 1,000 or less, and still further preferably 800 or less, for example, in the case where an organic layer containing the compound represented by the formula (1) is intended to be formed as a film by a vapor deposition method. The lower limit of the molecular weight is the molecular weight of the smallest compound represented by the formula (1).

The compound represented by the formula (1) may be formed into a film by a coating method irrespective of the molecular weight thereof. The compound that has a relatively large molecular weight may be formed into a film by a coating method.

As an application of the invention, a compound that contains plural structures each represented by the formula (1) in the molecule may be used as a lasing material.

For example, it may be considered that a polymerizable group is introduced in advance to the structure represented by the formula (1), and a polymer obtained by polymerizing the polymerizable group is used as a light-emitting material. Specifically, it may be considered that a monomer that has a polymerizable functional group at any of G¹, FL, BT, FL² and G² in the formula (1) is prepared, and is homopolymerized or copolymerized with another monomer to prepare a polymer containing repeating units, and the polymer is used as a lasing material. In alternative, it may be considered that the compounds containing a structure represented by the formula (1) are reacted to form a dimer or a trimer, and the dimer or the trimer is used as a light-emitting material.

Examples of the polymer having the repeating unit containing the structure represented by the formula (1) include a polymer containing a structure represented by the following formula (31) or (32).

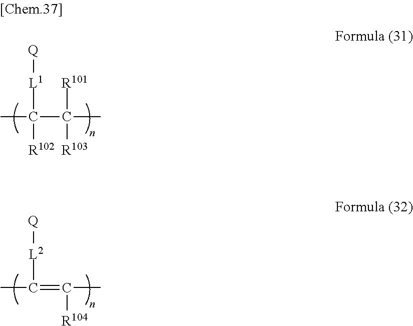

In the formulae (31) and (32), Q represents a group containing the structure represented by the formula (1), and L¹ and L² each represent a linking group. The linking group preferably has from 0 to 20 carbon atoms, more preferably from 1 to 15 carbon atoms, and further preferably from 2 to 10 carbon atoms. The linking group preferably has a structure represented by —X¹¹-L¹¹-, wherein X¹¹ represents an oxygen atom or a sulfur atom, and preferably an oxygen atom, and L¹¹ represents a linking group, preferably a substituted or unsubstituted alkylene group or a substituted or unsubstituted arylene group, and more preferably a substituted or unsubstituted alkylene group having from 1 to 10 carbon atoms or a substituted or unsubstituted phenylene group.

In the formulae (2) and (3), R¹⁰¹, R¹⁰², R¹⁰³ and R¹⁰⁴ each independently represent a substituent, preferably a substituted or unsubstituted alkyl group having from 1 to 6 carbon atoms, a substituted or unsubstituted alkoxy group having from 1 to 6 carbon atoms, or a halogen atom, more preferably an unsubstituted alkyl group having from 1 to 3 carbon atoms, an unsubstituted alkoxy group having from 1 to 3 carbon atoms, a fluorine atom or a chlorine atom, and further preferably an unsubstituted alkyl group having from 1 to 3 carbon atoms or an unsubstituted alkoxy group having from 1 to 3 carbon atoms.

The linking group represented by L¹ and L² may be bonded to any of G¹, FL¹, BT, FL 2 and G² of the structure of the formula (1) constituting Q. Two or more of the linking groups may be bonded to one group represented by Q to form a crosslinked structure or a network structure.

Specific examples of the structure of the repeating unit include structures represented by the following formulae (33) to (36).

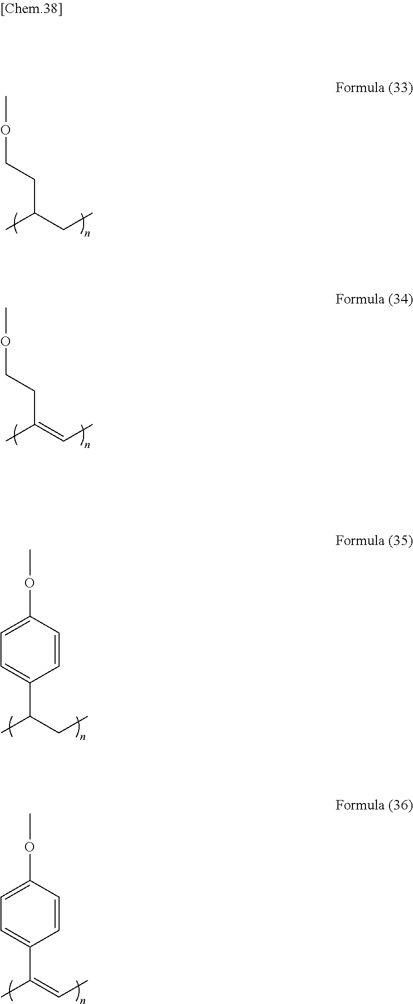

The polymer having the repeating unit containing the structure represented by any of the formulae (33) to (36) may be synthesized in such a manner that a hydroxyl group is introduced to any of G¹, FL¹, BT, FL² and G² of the formula (1), and the hydroxyl group as a linker is reacted with the following compound to introduce a polymerizable group thereto, followed by polymerizing the polymerizable group.

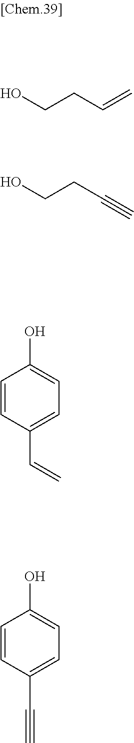

The polymer containing the structure represented by the formula (1) in the molecule may be a polymer containing only a repeating unit having the structure represented by the formula (1), or a polymer further containing a repeating unit having another structure. The repeating unit having the structure represented by the formula (1) contained in the polymer may be only one kind or two or more kinds. Examples of the repeating unit that does not have the structure represented by the formula (1) include a repeating unit derived from a monomer that is used for ordinary copolymerization. Examples of the repeating unit include a repeating unit derived from a monomer having an ethylenic unsaturated bond, such as ethylene and styrene.

Synthesis of Compound Represented by Formula (1)

The compounds represented by the formula (1) can be synthesized by known reactions. For example, they may be synthesized by the reaction schemes 1 to 4 shown in the Examples. The reaction conditions may be appropriately determined. For the details of the reactions, reference may be made to Synthesis Examples 1 to 3 below.

Organic Solid-State Laser

The present invention also provides an organic solid-state laser containing a compound represented by the formula (1). The term “organic solid-state laser” includes electrically-driven organic semiconductor laser diode (OSLD) device and optically-pumped solid-state laser device. A compound of the formula (1) is useful as a material used in a light-emitting layer (light amplification layer) of the organic solid-state laser. The light-emitting layer may contain two or more compounds of the formula (1) but preferably contains only one compound of the formula (1). The light-emitting layer may contain a host material. Preferable host material absorbs photo-excitation light for the organic solid-state laser. Another preferable host material has sufficient spectral overlap between its fluorescence spectrum and the absorption spectrum of the compound of the formula (1) contained in the light-emitting layer so that an effective Foerster-type energy transfer can take place from the host material to the compound of the formula (1). The concentration of the compound of the formula (1) in the light-emitting layer is preferably at least 0.1 wt %, more preferably at least 1 wt %, still more preferably at least 3 wt %, and preferably at most 50 wt %, more preferably at most 30 wt %, still more preferably at most 10 wt %.

The organic solid-state laser of the present invention has an optical resonator structure. The optical resonator structure may be a one-dimensional resonator structure or a two-dimensional resonator structure. Examples of the latter include a circulator resonator structure, and a whispering gallery type optical resonator structure. A distributed feedback (DFB) structure and a distributed Bragg reflector (DBR) structure are also employable. In a preferred embodiments, a second-order DFB is employed. For DFB, a mixed-order DFB grating structure may be employed. Namely, a mixed structure of DFB grating structures differing in point of the order relative to laser emission wavelength may be employed. Specific examples thereof include an optical resonator structure composed of a second-order Bragg scattering region adjacent to the first-order Bragg scattering region and a mixed structure where a second-order Bragg scattering region and a first-order scattering region are formed alternately. For details of preferred optical resonator structures, specific examples to be given hereinunder may be referred to. As the optical resonator structure, the organic solid-state laser may be further provided with an external optical resonator structure. For example, the optical resonator structure may be formed preferably on a glass substrate. The material to constitute the optical resonator structure includes an insulating material such as SiO₂, etc. For example, a grating structure is formed, the depth of the grating is preferably 75 nm or less, and is more preferably selected from a range of 10 to 75 nm. The depth may be, for example, 40 nm or more, or may be less than 40 nm. The light-emitting layer (light amplification layer) containing a compound of the formula (1) can be directly formed on the optical resonator structure.

The organic solid-state laser is preferably encapsulated by a sapphire or other materials to lower the lasing threshold and optimize the heat dissipation under intense optical pumping. An interlayer may be formed between the sapphire lid and the light-emitting layer. For example, amorphous fluorinated polymer such as CYTOP (trademark) is preferably used in the interlayer.

The grating may be placed so that at least one of the organic layer has a sufficient overlap between the distribution of exciton density and the electric field intensity distribution of the resonant optical mode, and/or so that the exciton may be excited efficiently.

The compound represented by the formula (1) could exhibit TADF properties, resulting in possible TADF-solid state laser.

Other advantages and features of the present invention may be better understood with respect to the following examples given for illustrative purposes and the accompanying figures.

### EXAMPLES

The invention will be described more specifically with reference to synthesis examples and working examples below. The materials, processes, procedures and the like shown below may be appropriately modified unless they deviate from the substance of the invention. Accordingly, the scope of the invention is not construed as being limited to the specific examples shown below.

The light emission characteristics were evaluated by using a high-performance UV/Vis/NIR spectrophotometer (Lambda 950, produced by PerkinElmer, Co., Ltd.), a fluorescence spectrophotometer (FluoroMax-4, produced by Horiba, Ltd.), an absolute PL quantum yield measurement system (C11347, produced by Hamamatsu Photonics K.K.), a source meter (2400 Series, produced by Keithley Instruments Inc.), a semiconductor parameter analyzer (E5273A, produced by Agilent Technologies, Inc.), an optical power meter (1930C, produced by Newport Corporation), an optical spectrometer (USB2000, produced by Ocean Optics, Inc.), a spectroradiometer (SR-3, produced by Topcon Corporation), and a streak camera (Model C4334, produced by Hamamatsu Photonics K.K.).

Syntheses

### (Synthesis Example 1) Synthesis of Compound 1

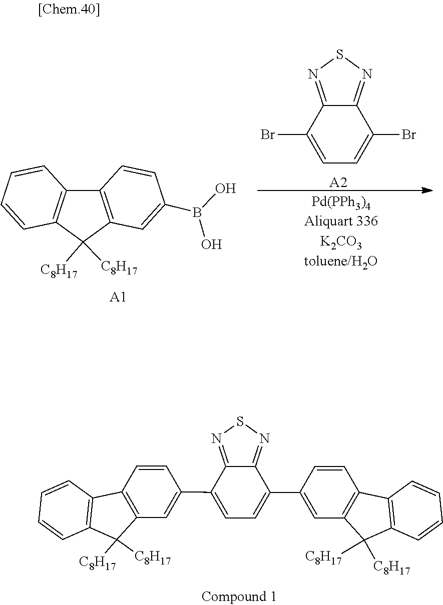

Compound A1 (2.4 eq), Compound A2 (1 eq), Pd(PPh₃)₄ (0.03 eq.), Aliquat 336 (0.05 eq), aqueous K₂CO₃ (8 eq, 1 M), and toluene (triple volume) were placed in a round bottom flask. The reaction mixture was stirred at 110° C. under nitrogen for 24 h. After cooling to room temperature, the mixture was extracted with toluene, dried over Na₂SO₄ and the solvent was removed. The crude product was purified by column chromatography over silica gel using hexane/DCM (3:1) mixture to give Compound 1 as a yellow solid (99%).

Compound 1

Mp 78-85° C. ¹H-NMR (CDCl₃, 500 MHz, ppm) δ: 8.03 (d, 2H, J=7.8 Hz); 7.95 (s, 2H); 7.89 (s, 2H); 7.88 (d, 2H); 7.78 (d, 2H, J=7.2 Hz); 7.40-7.33 (m, 6H); 2.10-1.98 (m, 8H); 1.22-1.16 (m, 8H); 1.15-1.09 (m, 32H); 0.80 (t, 12H, J=7.3 Hz); 0.78-0.73 (m, 8H). ¹³C NMR (CDCl₃, 125 MHz, ppm) δ: 154.51, 151.47, 151.26, 141.48, 140.81, 136.33, 133.75, 128.28, 128.04, 127.40, 126.99, 124.05, 123.11, 120.09, 119.84, 55.37, 40.44, 31.96, 30.23, 29.39, 29.37, 24.04, 22.75, 14.21. Anal. Calcd. For C₆₄H₈₄N₂S: C, 84.15; H, 9.27; N, 3.07; S, 3.51. Found: C, 84.15; H, 9.26; N, 3.00.

### (Synthesis Example 2) Synthesis of Compound 2

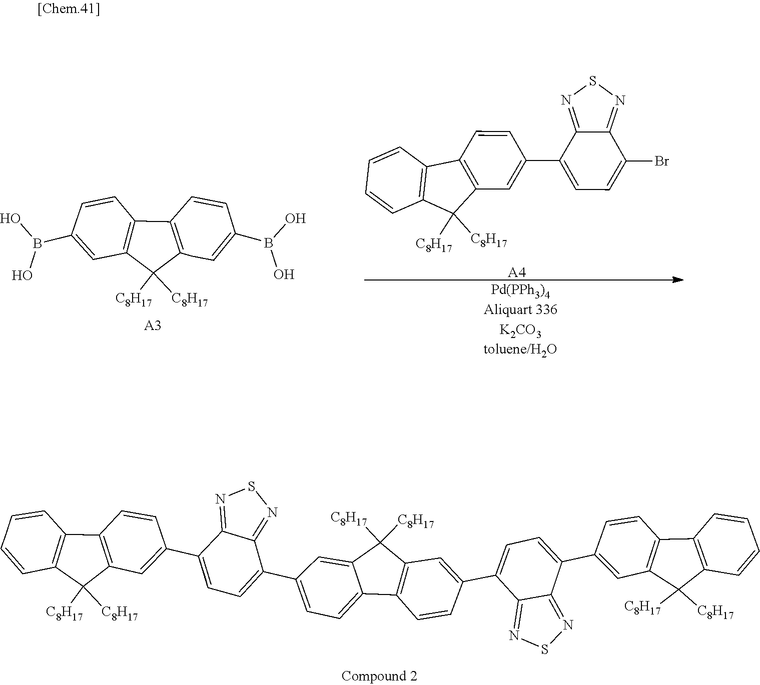

Compound A3 (0.302 g, 0.63 mmol), Compound A4 (0.787 g, 1.30 mmol), Pd(PPh₃)₄ (0.022 g, 0.019 mmol), Aliquat 336 (0.053 g, 0.13 mmol), K₂CO₃ (0.72 g, 5.20 mmol), H₂O (5 ml), and toluene (15 ml) were placed in a round bottom flask. The reaction mixture was stirred at 110° C. under nitrogen for 24 h. After cooling to room temperature, the mixture was extracted with toluene, dried over Na₂SO₄ and the solvent was removed. The crude product was purified by column chromatography over silica gel using hexane/DCM (3:1) mixture as eluent (R_{f}=0.20) to give Compound 2 as a yellow solid (0.83 g, 92%).

Compound 2

Mp 108-113° C. ¹H-NMR (CDCl₃, 500 MHz, ppm) δ: 8.08 (d, 2H, J=7.8 Hz); 8.05 (d, 2H, J=7.9 Hz); 8.01 (s, 2H); 7.97 (s, 2H); 7.96 (d, 2H, J=7.9 Hz); 7.93-7.88 (m, 6H); 7.79 (d, 2H, J=7.1 Hz); 7.41-7.33 (m, 6H); 2.15-1.99 (m, 12H); 1.22-1.10 (m, 60H); 0.97-0.92 (m, 4H); 0.82-0.75 (m, 26H). ¹³C NMR (CDCl₃, 125 MHz, ppm) δ: 154.49, 154.47, 151.87, 151.43, 151.23, 141.47, 140.97, 140.76, 136.59, 136.27, 133.80, 133.64, 128.41, 128.25, 128.07, 128.01, 127.37, 126.95, 124.12, 124.01, 123.08, 120.14, 120.06, 119.81, 55.54, 55.34, 40.40, 40.33, 31.95, 31.92, 30.23, 30.19, 29.38, 29.35, 29.34, 24.17, 24.01, 22.71, 14.18. Anal. Calcd. For C₉₉H₁₂₆N₄S₂: C, 82.79; H, 8.84; N, 3.90; S, 4.46. Found: C, 82.80; H, 8.80; N, 3.89.

### (Synthesis Example 3) Synthesis of Compound 3

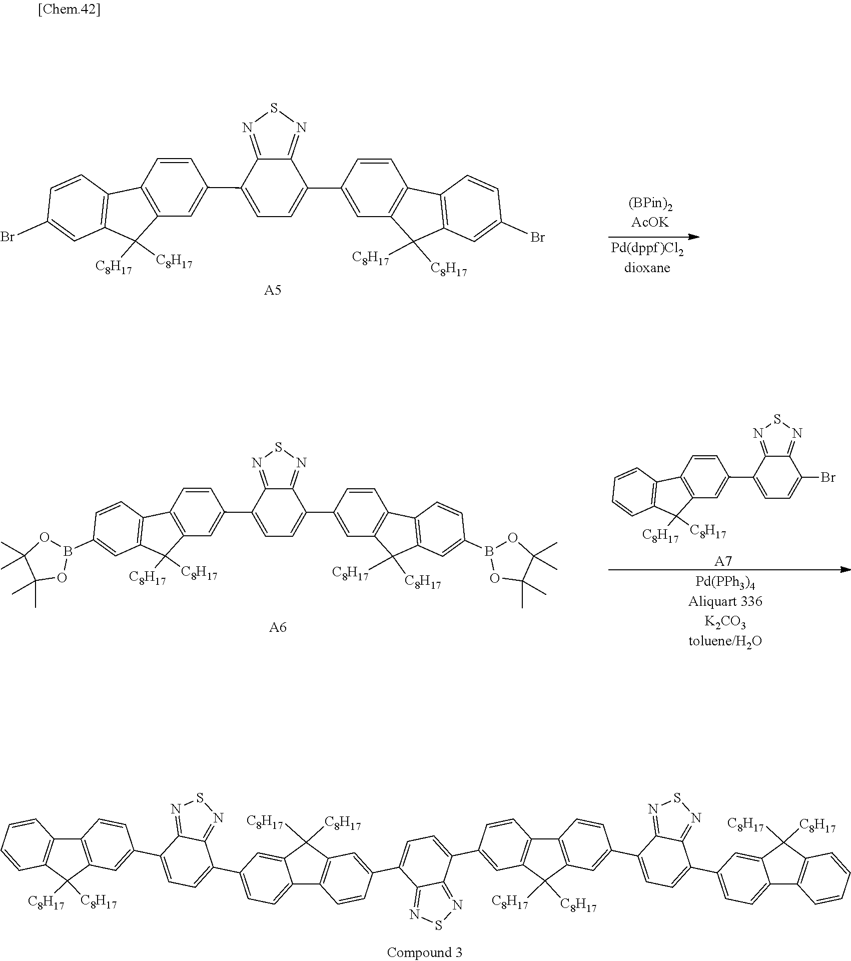

A mixture of Compound A5 (2.08 g, 1.94 mmol), potassium acetate (0.60 g, 6.10 mmol), bis(pinacolato)diboron (1.89 g, 7.45 mmol), and Pd(dppf)Cl₂.DCM (0.158 g, 0.19 mmol) in dry 1,4-dioxane (60 mL) was heated to 90° C. and stirred for 24 h under Ar. The solvent was removed under vacuum. The residue was purified by column chromatography over silica gel using hexane/DCM (1:1) mixture as eluent (Rf=0.37) to give Compound A6 as a yellow solid (1.80 g, 80%).

¹H-NMR (CDCl₃, 500 MHz, ppm) δ: 8.04 (d, 2H, J=7.9 Hz); 7.95 (s, 2H); 7.90 (d, 2H, J=7.9 Hz); 7.89 (s, 2H); 7.85 (d, 2H, J=7.5 Hz); 7.80 (s, 2H); 7.78 (d, 2H, J=7.5 Hz); 2.08-2.04 (m, 8H); 1.41 (s, 24H); 1.21-1.16 (m, 8H); 1.14-1.07 (m, 32H); 0.80 (t, 12H, J=7.2 Hz); 0.78-0.72 (m, 8H). ¹³C NMR (CDCl₃, 125 MHz, ppm) δ: 154.49, 151.88, 150.66, 143.79, 141.29, 136.81, 133.98, 133.78, 129.09, 128.28, 128.08, 124.13, 120.30, 119.41, 83.89, 55.45, 40.30, 31.96, 30.18, 29.39, 29.35, 25.11, 23.99, 22.75, 14.22. Anal. Calcd. For C₇₆H₁₀₆B₂N₂O₄S: C, 78.33; H, 9.17; B, 1.86; N, 2.40; O, 5.49; S, 2.75. Found: C, 78.40; H, 9.18; N, 2.42.

Compound A6 (0.593 g, 0.51 mmol), compound A7 (0.779 g, 1.29 mmol), Pd(PPh₃)₄ (0.042 g, 0.036 mmol), Aliquat 336 (0.046 g, 0.11 mmol), K₂CO₃ (0.319 g, 2.31 mmol), H₂O (10 ml), and toluene (30 ml) were placed in a round bottom flask. The reaction mixture was stirred at 110° C. under nitrogen for 24 h. After cooling to room temperature, the mixture was extracted with toluene, dried over Na₂SO₄ and the solvent was removed. The crude product was purified by column chromatography over silica gel using hexane/DCM (2:1) mixture as eluent (R_{f}=0.23) to give Compound 3 as a yellow solid (0.98 g, 98%).

Compound 3

Mp 138-141° C. ¹H-NMR (CDCl₃, 500 MHz, ppm) δ: 8.10-8.08 (m, 4H); 8.05 (d, 2H, J=7.8 Hz); 8.03 (s, 2H); 8.02 (s, 2H); 8.00-7.88 (m, 14H); 7.79 (d, 2H, J=7.1 Hz); 7.41-7.33 (m, 6H); 2.16-2.13 (m, 8H); 2.10-1.99 (m, 8H); 1.21-1.10 (m, 80H); 0.97-0.93 (m, 8H); 0.82-0.75 (m, 32H). ¹³C NMR (CDCl₃, 125 MHz, ppm) δ: 154.53, 151.93, 151.48, 151.28, 141.52, 141.05, 141.02, 140.81, 136.65, 136.62, 136.31, 133.85, 133.78, 133.68, 128.47, 128.30, 128.12, 128.05, 127.43, 127.00, 124.18, 124.06, 123.12, 120.20, 120.11, 119.86, 55.60, 55.38, 40.45, 40.38, 32.00, 31.97, 30.28, 30.24, 29.44, 29.40, 29.38, 24.22, 24.05, 22.77, 22.76, 14.22. Anal. Calcd. For C₁₃₄H₁₆₈N₆S₃: C, 82.16; H, 8.64; N, 4.29; S, 4.91. Found: C, 82.08; H, 8.63; N, 4.25.

### (Synthesis Examples 4 to 36) Synthesis of Compounds 4 to 36

In a similar way, Compounds 4 to 36 were synthesized by the following schemes:

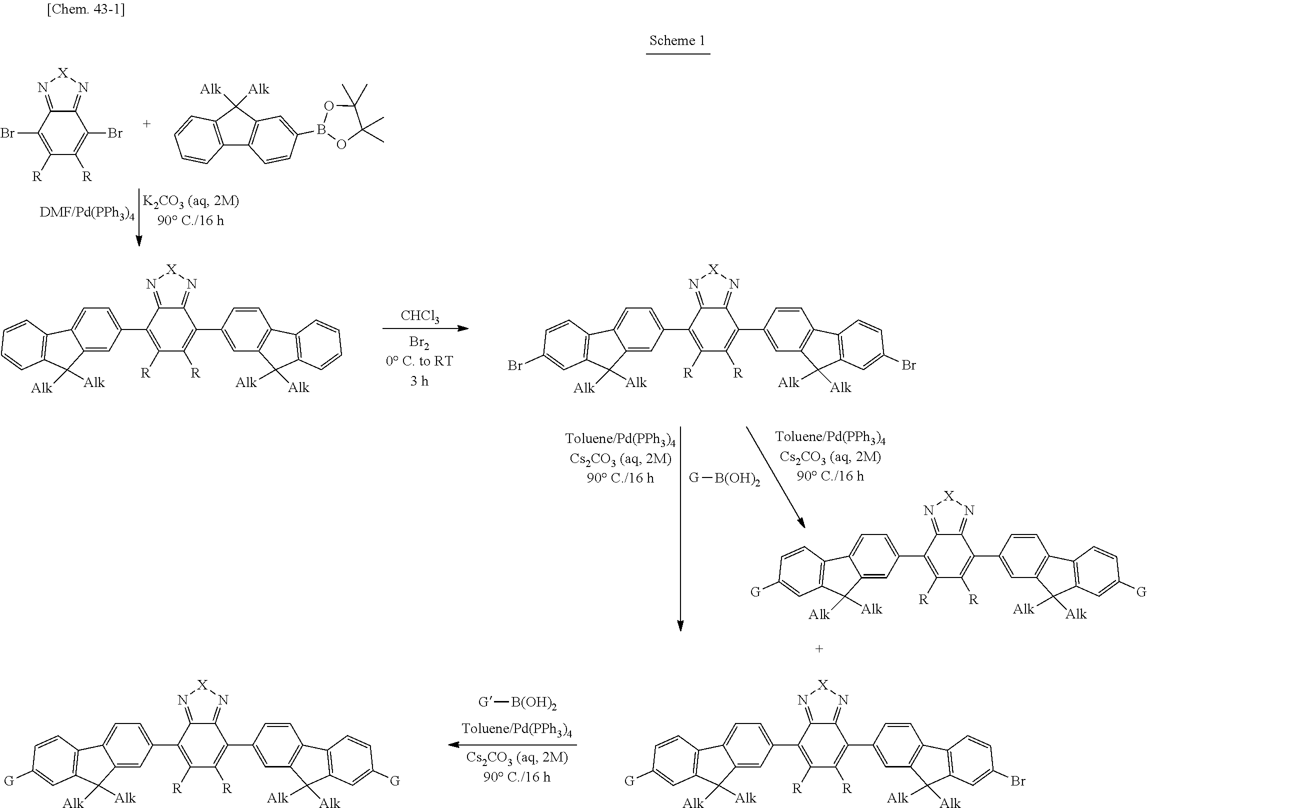

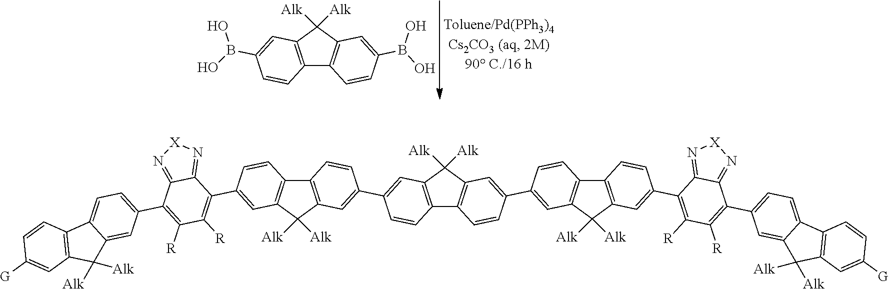

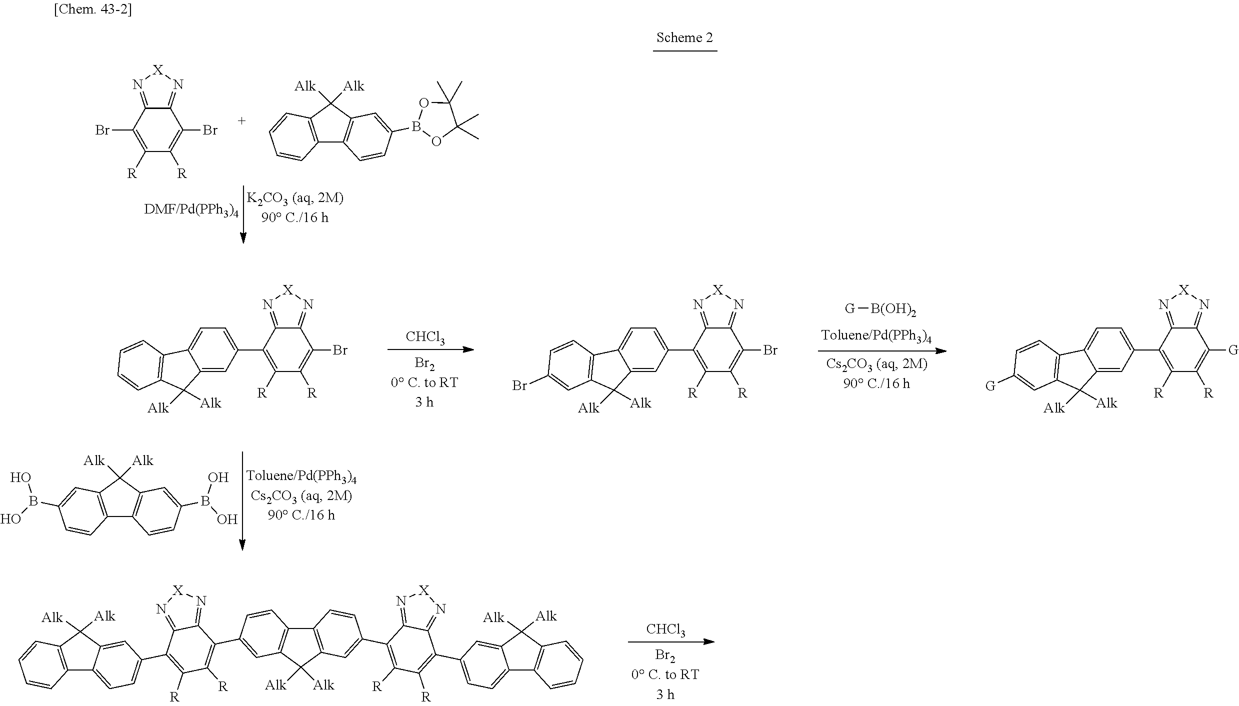

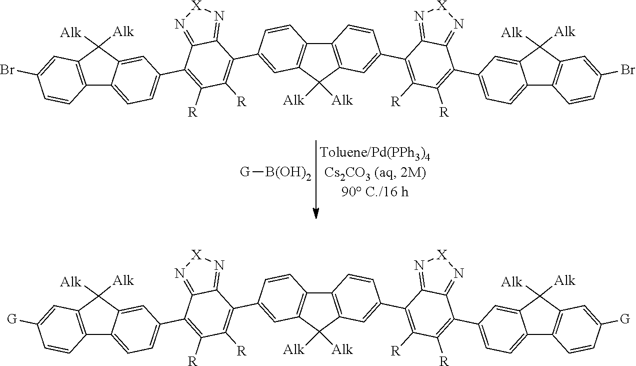

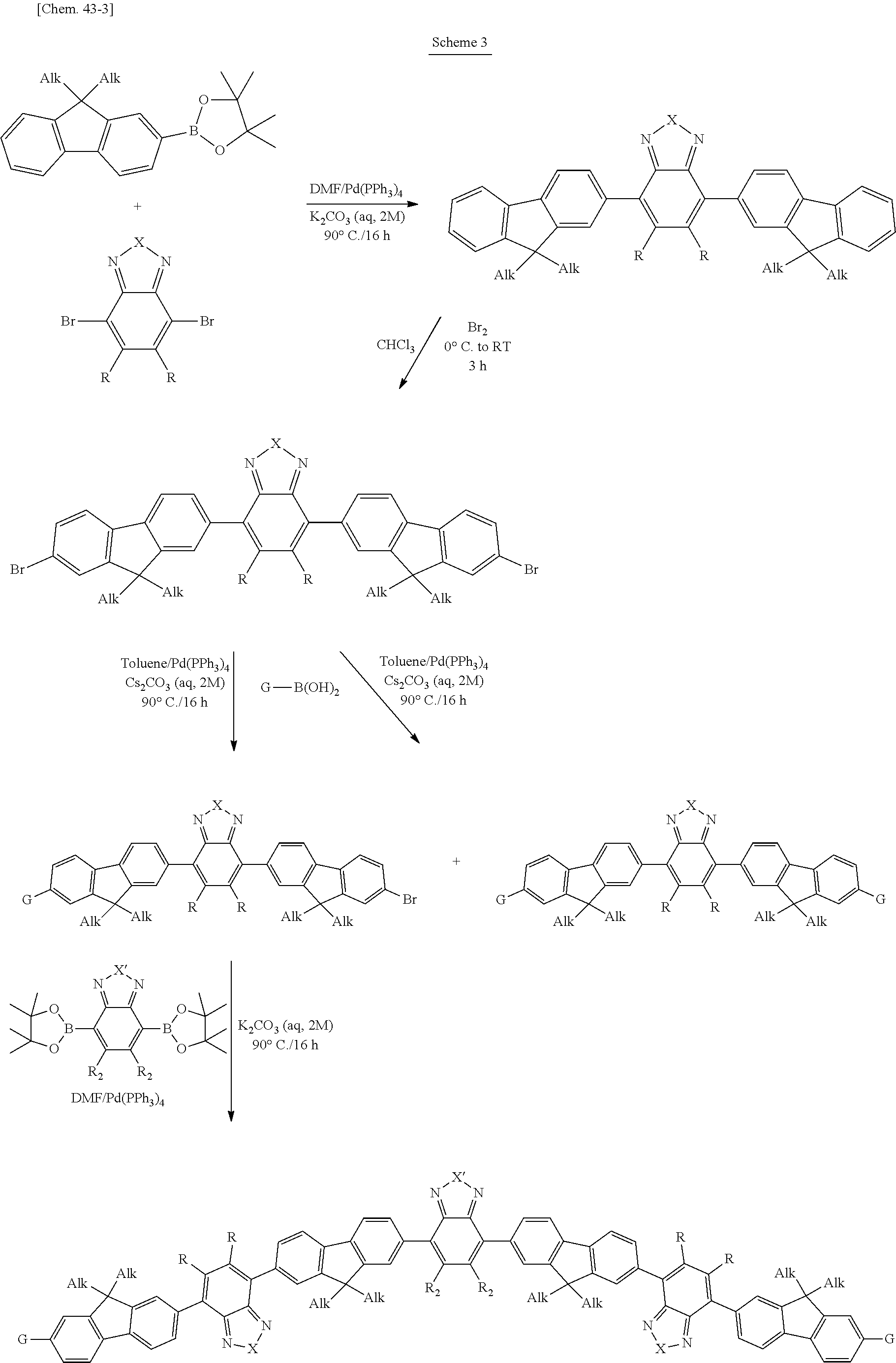

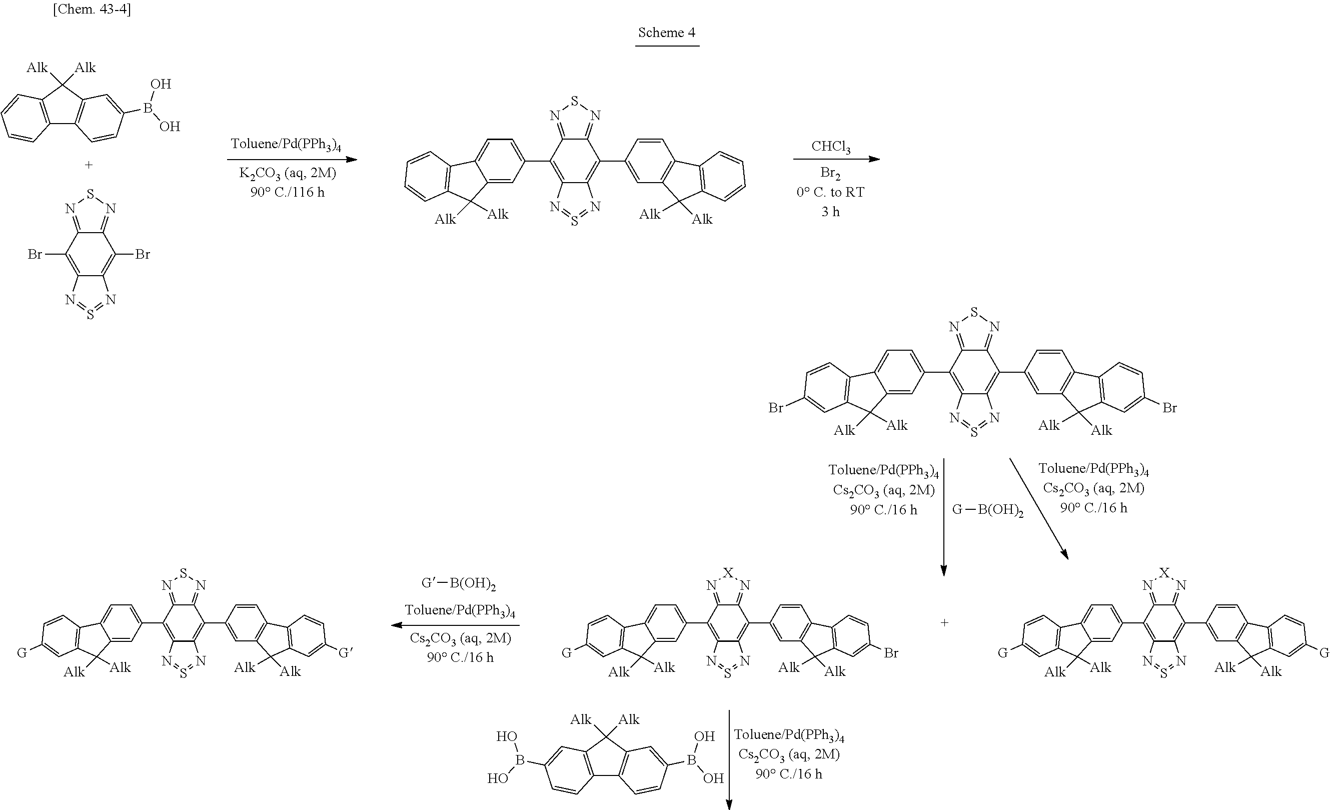

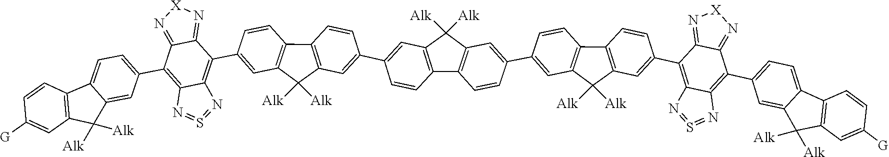

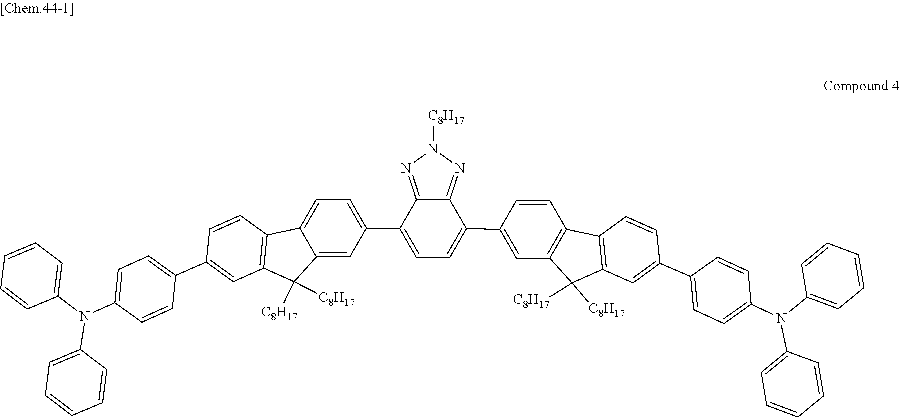

¹H NMR (CD₂Cl₂, 300 MHz, ppm) δ: 7.93-7.87 (m, 4H), 7.79-7.70 (m, 4H), 7.68-7.64 (m, 8H), 7.37-7.31 (m, 8H), 7.23-7.17 (m, 12H), 7.12-7.08 (m, 4H), 3.93 (t, J=6.7 Hz, 4H), 2.15-2.10 (m, 8H), 1.66-1.61 (m, 4H), 1.24-1.16 (m, 62H), 0.90-0.82 (m, 26H); ¹³C NMR (CD₂Cl₂, 75 MHz, ppm) δ: 153.71, 153.05, 152.59, 151.07, 148.30, 147.72, 141.03, 140.40, 140.16, 136.14, 133.32, 130.37, 129.85, 128.32, 126.27, 126.03, 125.33, 124.94, 124.53, 123.53, 121.63, 120.67, 119.62, 75.05, 55.84, 40.87, 32.42, 30.85, 30.71, 30.00, 29.92, 29.88, 26.58, 24.64, 23.25, 23.19, 14.42.

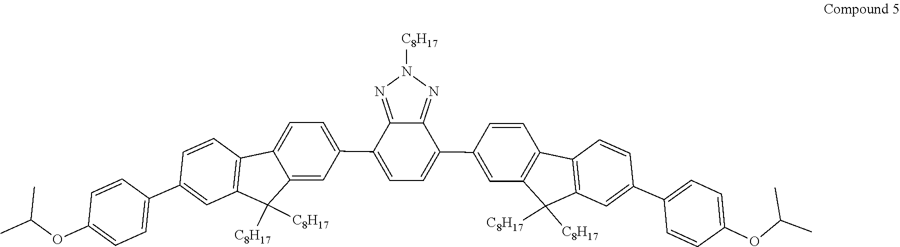

¹H NMR (CDCl₃, 300 MHz, ppm) δ: 8.18 (dd, ³J=7.8 Hz, ⁴J=1.2 Hz, 2H), 8.10 (s, 2H), 7.89 (d, ³J=8.1 Hz, 2H), 7.80 (m, 4H), 7.62 (m, 8H), 7.04 (d, ³J=8.7 Hz, 4H), 4.86 (t, ³J=7.2 Hz, 2H), 4.66 (m, 2H), 2.32-2.00 (m, 10H), 1.56-1.27 (m, 20H), 1.26-1.09 (m, 42H), 0.96-0.78 (m, 23H); ¹³C NMR (CDCl₃, 75 MHz, ppm) δ: 157.41, 152.00, 151.32, 143.72, 140.72, 139.83, 139.53, 136.07, 134.09, 130.56, 128.22, 127.55, 125.59, 124.35, 123.18, 121.15, 120.07, 119.79, 116.18, 70.03, 55.23, 40.45, 31.82, 30.18, 29.32, 29.26, 29.17, 26.78, 23.96, 22.68, 22.62, 22.13, 14.12, 14.08.

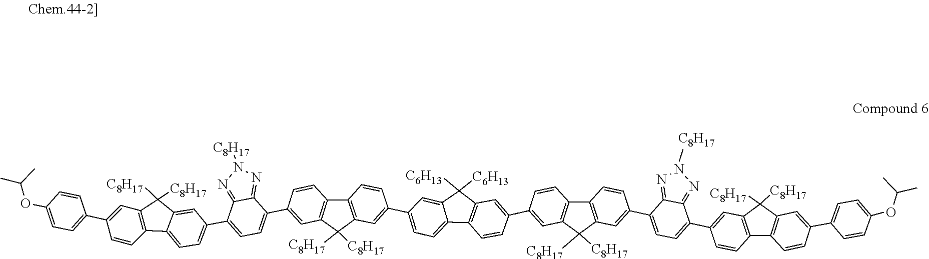

¹H NMR (CDCl₃, 300 MHz, ppm) δ: 8.20-8.14 (m, 4H), 8.12-8.07 (m, 4H), 7.94-7.83 (m, 8H), 7.82-7.76 (m, 6H), 7.74-7.68 (m, 8H), 7.63 (d, ³J=8.7 Hz, 4H), 7.59-7.54 (m, 4H), 7.01 (d, ³J=8.7 Hz, 4H), 4.85 (t, ³J=6.9 Hz, 4H), 4.62 (m, 2H), 2.30-2.02 (m, 24H), 1.54-1.26 (m, 28H), 1.25-1.06 (96H), 1.00-0.75 (m, 56H); 13C NMR (CDCl₃, 75 MHz, ppm) δ: 157.43, 152.17, 152.02, 151.85, 151.46, 151.34, 143.75, 140.77, 140.64, 140.57, 140.11, 140.07, 139.86, 139.54, 136.28, 136.08, 134.10, 130.65, 130.55, 128.24, 127.62, 127, 58, 126.22, 125.62, 124.42, 124.38, 123.31, 123.22, 121.56, 121.16, 120.10, 120.01, 119.95, 119.82, 116.19, 70.03, 56.87, 55.39, 55.34, 55.25, 40.48, 40.44, 31.85, 31.52, 30.21, 30.13, 29.72, 29.34, 29.29, 29.20, 26.81, 24.06, 23.99, 23.89, 22.70, 22.65, 22.61, 22.14, 14.14, 14.10.

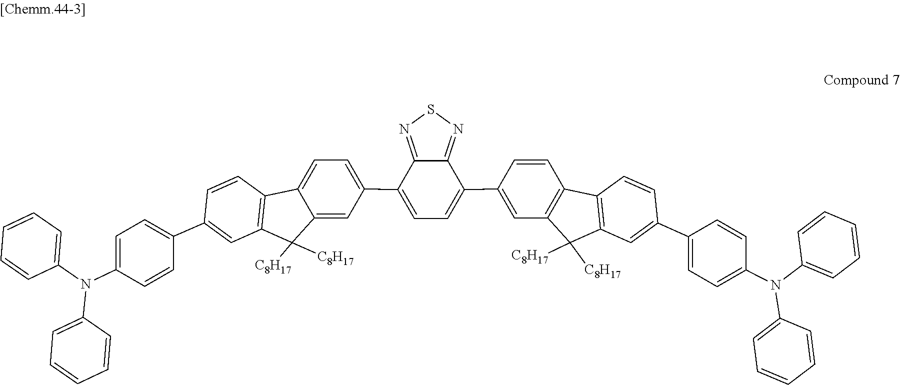

¹H NMR (CDCl₃, 300 MHz, ppm) δ: 8.10 (dd, J=7.9 Hz, 2H), 8.02 (s, 2H), 7.93-7.90 (m, 4H), 7.84 (d, J=8.3 Hz, 2H), 7.64-7.61 (m, 8H), 7.34-7.29 (m, 8H), 7.24-7.18 (m, 12H), 7.10-7.05 (m, 4H), 2.16-2.09 (m, 8H), 1.23-1.14 (m, 40H), 0.90-0.80 (m, 20H); ¹³C NMR (CDCl₃, 75 MHz, ppm) δ: 154.51, 152.16, 151.48, 147.82, 147.24, 141.22, 139.88, 139.68, 136.22, 135.71, 133.69, 129.42, 128.37, 128.01, 127.96, 125.76, 124.51, 124.15, 124.04, 123.06, 121.15, 120.37, 119.96, 55.45, 40.50, 31.94, 30.22, 29.39, 29.36, 24.07, 22.74, 14.21.

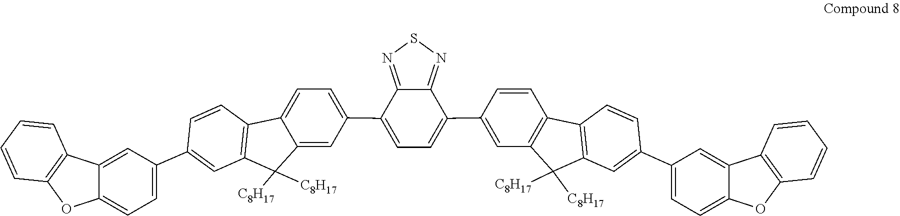

¹H NMR (CDCl₃, 300 MHz, ppm) δ: 8.12-8.10 (dd, J=7.8 Hz, 2H), 8.05-7.95 (m, 16H), 7.75-7.72 (dd, J=8.7 Hz, 2H), 7.65-7.63 (d, J=8.1 Hz, 2H), 7.52-7.47 (m, 4H), 7.43-7.37 (m, 2H), 2.19-2.14 (m, 8H), 1.21-1.16 (m, 44H), 0.99-0.94 (m, 12H), 0.82-0.78 (m, 12H); ¹³C NMR (CDCl₃, 75 MHz, ppm) δ: 156.33, 154.53, 153.62, 151.90, 151.82, 141.17, 140.47, 136.48, 135.51, 133.75, 128.43, 128.10, 127.91, 127.33, 126.98, 126.61, 125.14, 124.43, 14.13, 123.65, 13.38, 122.93, 120.87, 120.27, 120.09, 119.85, 111.98, 55.54, 40.43, 31.96, 30.31, 29.45, 29.41, 24.19, 22.75, 14.20.

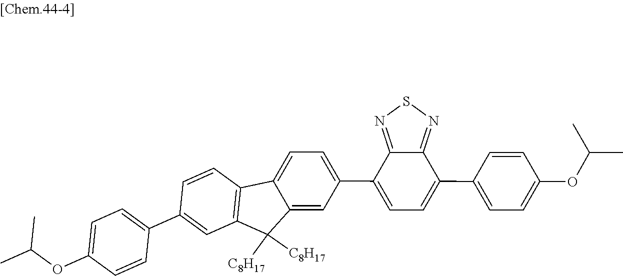

¹H NMR (CDCl₃, 300 MHz, ppm) δ: 8.04 (dd, ³J=7.8 Hz, ⁴J=1.5 Hz, 1H), 8.00-7.98 (m, 3H), 7.89-7.85 (m, 2H), 7.83, 7.76 (m, 2H), 7.64 (d, ³J=8.7 Hz, 2H), 7.61-7.57 (m, 2H), 7.09 (d, ³J=9.0 Hz, 2H), 7.03 (d, ³J=9.0 Hz, 2H), 4.72-4.60 (m, 2H), 2.15-2.05 (m, 4H), 1.45, 1.40 (m, 12H), 1.25-1.12 (m, 20H), 0.92-0.79 (m, 10H); ¹³C NMR (CDCl₃, 75 MHz, ppm) δ: 158.34, 157.55, 154.44, 154.40, 152.09, 151.40, 141.16, 140.10, 139.46, 136.14, 134.10, 133.26, 132.81, 130.55, 129.83, 128.32, 128.04, 127.42, 125.74, 123.96, 121.24, 120.29, 119.77, 116.27, 115.98, 70.08, 55.42, 40.49, 31.93, 30.19, 29.36, 29.33, 24.03, 22.72, 22.26, 22.23, 14.19.

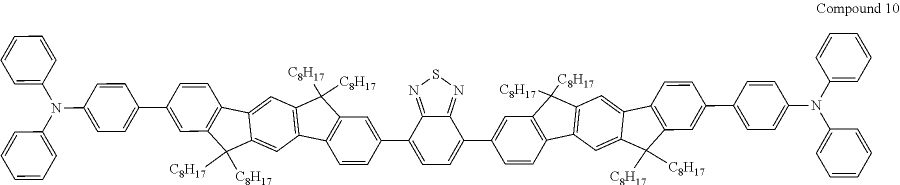

¹H NMR (CD₂Cl₂, 300 MHz, ppm) δ: 8.14-8.10 (m, 4H), 7.97-7.95 (m, 4H), 7.87-7.79 (m, 6H), 7.66-7.63 (m, 8H) 7.34-7.29 (m, 8H), 7.21-7.15 (m, 12H), 7.10-7.05 (m, 4H), 2.21-2.14 (m, 16H), 1.21-1.11 (m, 84H), 0.84-0.78 (m, 44H); ¹³C NMR (CD₂Cl₂, 75 MHz, ppm) δ: 155.01, 152.58, 152.10, 151.45, 151.12, 148.34, 147.69, 142.38, 141.29, 141/06, 140.75, 139.73, 136.51, 136.21, 134.00, 129.88, 128.84, 128.40, 128.28, 125.91, 124.93, 124.83, 123.53, 121.52, 120.32, 119.91, 114.92, 114.69, 55.59, 55.53. 41.28, 41.15, 32.45, 32.43, 30.64, 29.99, 29.83, 29.82, 24.56, 24.51, 23.21, 14.47.

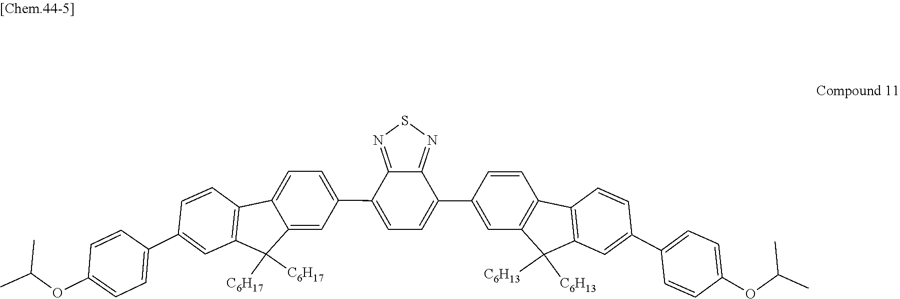

¹H NMR (CDCl₃, 300 MHz, ppm) δ: 8.08 (dd, ³J=7.8 Hz, ⁴J=1.5 Hz, 2H), 8.32 (d, ⁴J=0.9 Hz, 2H), 7.95-7.90 (m, 2H), 7.84 (d, ³J=8.7 Hz, 2H), 7.64 (d, ³J=8.7 Hz, 4H), 7.63-7.56 (m, 4H), 7.05 (d, ³J=9.0 Hz, 4H), 4.66 (m, 2H), 4.21-4.03 (m, 8H), 1.44 (s, 6H), 1.42 (s, 6H), 1.20-1.12 (m, 24H), 0.91-0.77 (m, 20H);

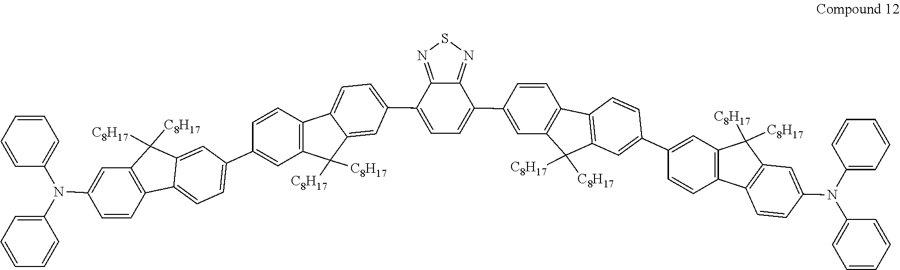

¹H NMR (CD₂Cl₂, 300 MHz, ppm) δ: 8.20-8.14 (m, 4H), 8.05-7.95 (m, 6H) 7.85-7.70 (m, 12H), 7.40-7.32 (m, 8H), 7.29-7.19 (m, 10H), 7.15-7.08 (m, 6H), 2.32-1.93 (m, 16H), 1.38-1.15 (m, 80H), 1.00-0.78 (m, 40H); ¹³C NMR (CD₂Cl₂, 75 MHz, ppm) δ: 154.96, 153.07, 152.74, 152.10, 151.96, 148.64, 147.84, 141.62, 141.42, 140.88, 140.37, 140.25, 136.84, 136.58, 134.02, 129.75, 128.93, 128.48, 126.67, 126.65, 124.65, 124.39, 124.13, 123.09, 122.05, 121.90, 120.96, 120.78, 120.22, 120.09, 119.91, 56.02, 55.82, 40.84, 32.41, 32.40, 30.62, 30.59, 29.90, 29.83, 24.60, 24.55, 23.22, 23.19, 14.46, 14.42.

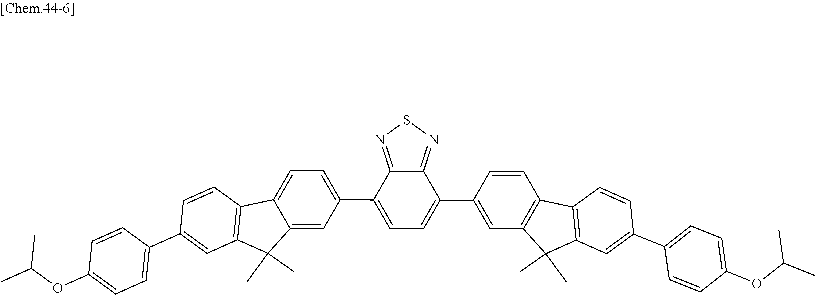 [SMILES: CC(C)Oc1ccc(-c2ccc3c(c2)C(C)(C)c2cc(-c4ccc(-c5ccc6c(c5)C(C)(C)c5cc(-c7ccc(OC(C)C)cc7)ccc5-6)c5nsnc45)ccc2-3)cc1]

¹H NMR (CDCl₃, 300 MHz, ppm) δ: 8.10-8.02 (m, 4H), 7.96-7.90 (m, 4H), 7.86 (d, ³J=8.4 Hz, 2H), 7.70-7.55 (m, 8H), 7.02 (d, ³J=9.0 Hz, 4H), 4.65 (m, 2H), 1.67 (s, 12H), 1.43 (s, 6H), 1.41 (s, 6H);

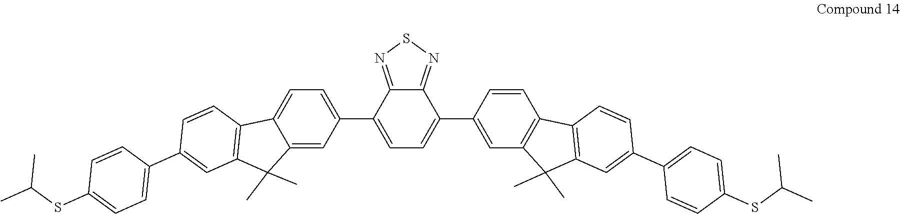 [SMILES: CC(C)Sc1ccc(-c2ccc3c(c2)C(C)(C)c2cc(-c4ccc(-c5ccc6c(c5)C(C)(C)c5cc(-c7ccc(SC(C)C)cc7)ccc5-6)c5nsnc45)ccc2-3)cc1]

¹H NMR (CDCl₃, 300 MHz, ppm) δ: 8.08-8.01 (m, 4H), 7.95-7.89 (m, 4H), 7.86 (d, ³J=7.8 Hz, 2H), 7.68 (d, ⁴J=1.2 Hz, 2H), 7.66-7.59 (m, 6H), 7.50 (d, ³J=8.4 Hz, 4H), 3.45 (m, 2H), 1.65 (s, 6H), 1.37 (s, 6H), 1.34 (s, 6H);

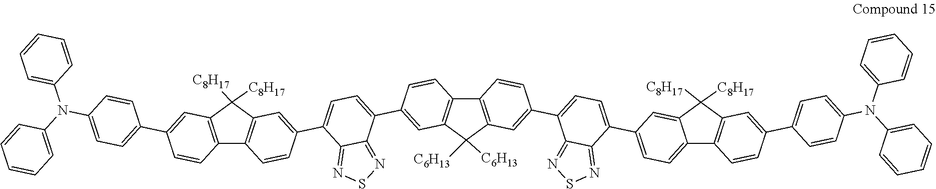

¹H NMR (CDCl₃, 300 MHz, ppm) δ: 8.17-8.00 (m, 8H), 8.05-7.98 (m, 6H), 7.94 (d, ³J=8.4 Hz, 2H), 7.89 (d, ³J=7.8 Hz, 2H), 7.70-7.62 (m, 8H), 7.38-7.30 (m, 8H), 7.25-7.16 (m, 12H), 7.15-7.07 (m, 4H), 2.22-2.15 (m, 12H), 1.23-1.15 (m, 52H), 0.96-0.80 (m, 30H); ¹³C NMR (CDCl₃, 75 MHz, ppm) δ: 154.95, 152.70, 152.30, 151.92, 148.29, 147.77, 141.64, 141.40, 140.35, 140.15, 137.17, 136.77, 136.04, 134.08, 133.94, 129.87, 128.98, 128.93, 128.54, 128.47, 128.33, 126.09, 124.96, 124.75, 124.62, 124.51, 123.55, 121.66, 120.83, 120.52, 120.19, 56.05, 55.96, 40.85, 40.77, 32.40, 32.18, 32.14, 30.62, 30.36, 29.82, 24.56, 23.24, 23.19, 14.48, 14.42.

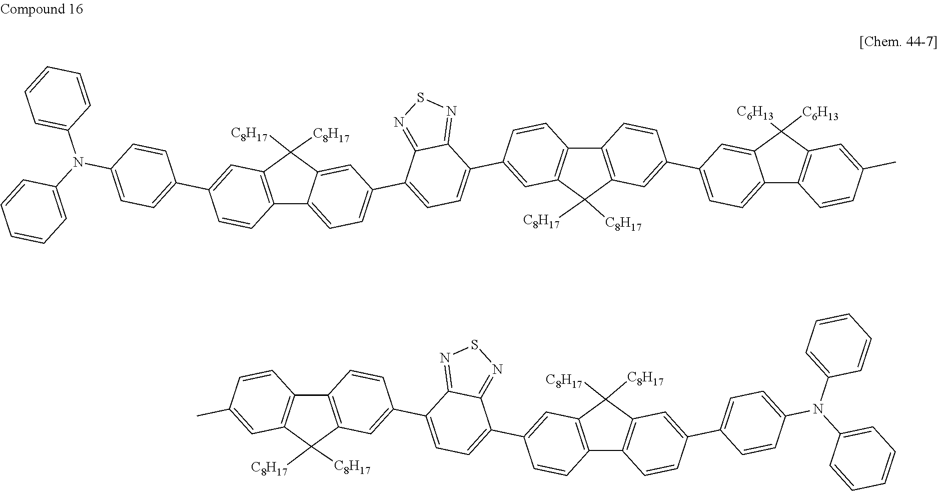

¹H NMR (CD₂Cl₂, 300 MHz, ppm) δ: 8.11-8.06 (m, 8H), 7.95-7.84 (m, 14H), 7.75 (m, 8H), 7.65-7.61 (m, 8H), 7.30 (m, 8H), 7.15 (m, 12H), 7.07 (m, 4H), 2.16 (m, 20H), 1.16 (m, 92H), 0.82 (m, 50H); ¹³C NMR (CD₂Cl₂, 75 MHz, ppm) δ: 154.38, 152.20, 152.13, 151.88, 151.42, 151.35, 147.72, 147.21, 141.06, 141.01, 140.81, 140.50, 140.14, 139.93, 139.77, 139.58, 136.32, 136.21, 135.47, 133.45, 133.41, 129.30, 128.36, 127.92, 127.76, 126.19, 125.52, 124.39, 124.10, 124.03, 123.94, 122.99, 121.59, 121.09, 120.25, 120.01, 119.70, 119.60, 55.46, 55.38, 40.39, 40.29, 31.83, 31.53, 30.05, 29.68, 29.25, 24.04, 23.99, 22.62, 22.59, 13.86, 13.83.

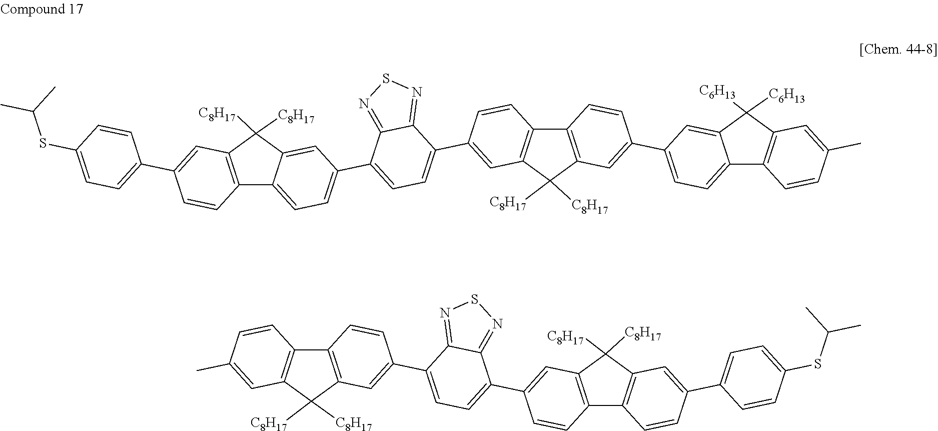

¹H NMR (CDCl₃, 300 MHz, ppm) δ: 8.14-8.07 (m, 4H), 8.07-8.01 (m, 4H), 7.99-7.83 (m, 14H), 7.80-7.71 (m, 8H), 7.70-7.61 (m, 8H), 7.54 (d, ³J=8.4 Hz, 4H), 3.47 (m, 2H), 2.35-2.01 (m, 20H), 1.39 (s, 6H), 1.37 (s, 6H), 1.28-1.12 (m, 92H), 1.00-0.78 (m, 50H); ¹³C NMR (CDCl₃, 75 MHz, ppm) δ: 154.44, 154.43, 152.20, 152.15, 151.88, 151.50, 151.45, 141.10, 140.98, 140.87, 140.54, 140.16, 140.13, 140.08, 139.92, 139.57, 136.33, 136.24, 134.52, 133.67, 133.60, 132.30, 128.33, 127.96, 127.53, 126.29, 125.99, 124.06, 124.00, 121.59, 121.38, 120.35, 120.30, 120.06, 119.88, 55.44, 55.42, 40.38, 38.39, 31.87, 31.53, 30.14, 30.12, 29.74, 29.29, 29.26, 24.06, 23.98, 23.22, 22.66, 22.63, 14.13, 14.10.

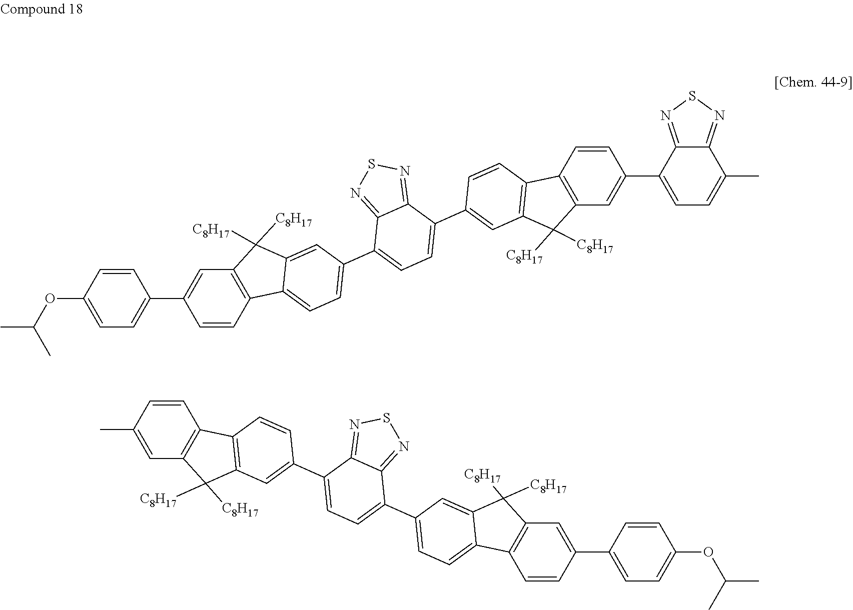

¹H NMR (CDCl₃, 300 MHz, ppm) δ: 8.03 (m, 24H), 7.85 (d, ³J=8.4 Hz, 2H), 7.64 (m, 8H), 7.05 (d, ³J=8.7 Hz, 4H), 4.67 (m, 2H), 2.18 (m, 8H), 1.45 (s, 6H), 1.43 (s, 6H), 1.20 (m, 84H), 0.93 (m, 44H); ¹³C NMR (CDCl₃, 75 MHz, ppm) δ: 157.47, 154.42, 152.04, 151.82, 151.38, 141.21, 140.95, 140.90, 141.08, 139.35, 134.03, 133.74, 133.65, 133.53, 128.38, 128.26, 128.03, 127.92, 125.69, 124.08, 123.96, 121.19, 120.24, 120.11, 119.73, 116.20, 70.03, 55.48, 55.35, 40.41, 40.29, 31.89, 31.86, 30.17, 30.13, 29.33, 29.30, 29.26, 24.12, 23.98, 22.66, 22.65, 22.15, 14.12.

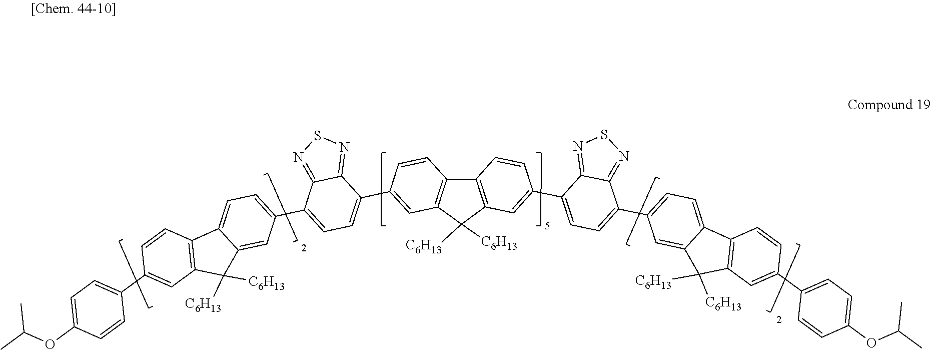

¹H NMR (CD₂Cl₂, 300 MHz, ppm) δ: 8.10 (m, 8H), 7.89 (m, 20H), 7.74 (m, 26H), 7.60 (m, 8H), 7.00 (d, ³J=8.7 Hz, 4H), 4.63 (m, 2H), 2.15 (m, 36H), 1.38 (s, 6H), 1.36 (s, 6H), 1.15 (m, 108H), 0.82 (m, 90H); ¹³C NMR (CD₂Cl₂, 75 MHz, ppm) δ: 157.53, 154.40, 152.20, 151.88, 151.77, 151.47, 141.03, 140.83, 140.52, 140.49, 140.29, 140.24, 140.15, 140.11, 140.09, 139.93, 139.87, 139.78, 139.47, 136.32, 136.28, 136.24, 133.46, 128.37, 128.08, 127.93, 126.14, 125.45, 124.11, 121.57, 121.55, 121.09, 120.25, 120.02, 119.68, 116.10, 69.96, 55.45, 55.34, 40.40, 40.29, 31.53, 29.72, 24.00, 23.92, 22.59, 21.85, 13.82.

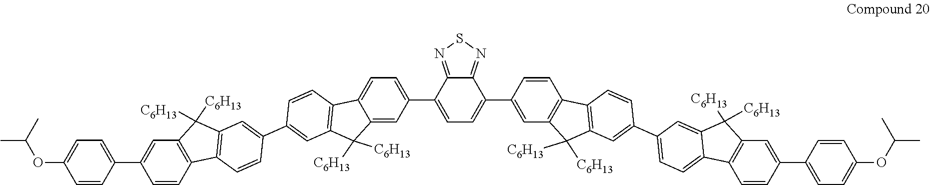

¹H NMR (CDCl₃, 300 MHz, ppm) δ: 8.08 (m, 4H), 7.92 (m, 6H), 7.82 (m, 4H), 7.66 (m, 16H), 7.04 (d, ³J=8.7 Hz, 4H), 4.65 (m, 2H), 2.15 (m, 16H), 1.43 (s, 6H), 1.41 (s, 6H), 1.17 (m, 48H), 0.89 (m, 40H); ¹³C NMR (CDCl₃, 75 MHz, ppm) δ: 155.55, 152.56, 150.28, 149.85, 149.83, 149.57, 139.20, 139.01, 138.45, 138.34, 137.97, 137.91, 137.60, 134.33, 132.17, 131.75, 126.42, 126.33, 126.05, 124.38, 124.30, 123.74, 122.18, 119.67, 119.60, 119.24, 118.38, 118.11, 118.03, 117.96, 114.31, 68.12, 53.53, 53.42, 38.62, 38.47, 29.64, 29.63, 27.89, 27.87, 22.10, 21.98, 20.74, 20.26, 12.20, 12.17.

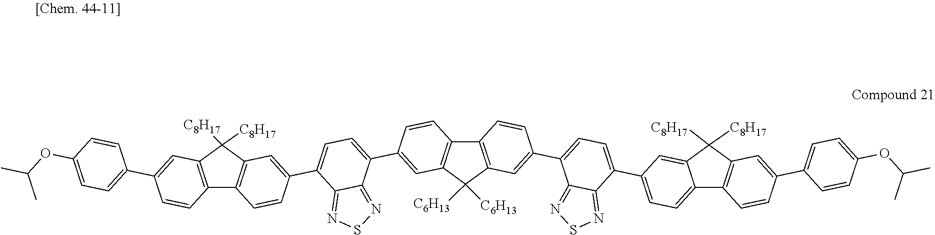

¹H NMR (CD Cl₃, 300 MHz, ppm) δ: 8.09 (m, 6H), 7.97 (m, 10H), 7.85 (d, ³J=8.7 Hz, 2H), 7.63 (m, 8H), 7.05 (d, ³J=8.7 Hz, 4H), 4.66 (m, 2H), 2.15 (m, 12H), 1.44 (s, 6H), 1.42 (s, 6H), 1.17 (m, 52H), 0.86 (m, 30H);

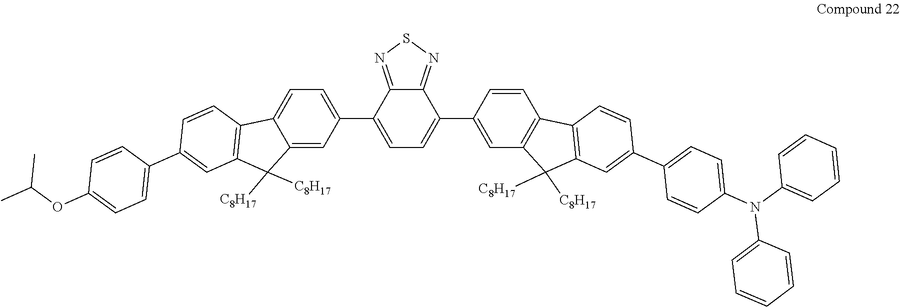

¹H NMR (CDCl₃, 300 MHz, ppm) δ: 8.09 (d, 3J=8.1 Hz, 2H), 8.01 (s, 2H); 8.94 (m, 4H), 7.84 (dd, ³J=8.7 Hz, ⁴J=2.1 Hz, 2H), 7.62 (m, 8H), 7.32 (4H), 7.20 (m, 6H), 7.07 (m, 4H), 4.66 (m, 1H), 2.12 (m, 8H), 1.44 (s, 3H), 1.42 (s, 3H), 1.14 (m, 40H), 0.90 (m, 20H); ¹³C NMR (CDCl₃, 75 MHz, ppm) δ: 157.49, 154.44, 152.08, 152.06, 152.04, 151.39, 147.75, 147.16, 141.19, 141.14, 140.09, 139.81, 139.59, 139.37, 136.15, 136.08, 135.63, 134.04, 133.64, 133.60, 129.35, 128.27, 127.93, 127.88, 125.68, 124.44, 124.07, 123.96, 122.98, 121.19, 121.07, 120.28, 120.25, 119.75, 116.21, 70.03, 55.36, 40.42, 31.87, 30.14, 29.29, 23.98, 22.67, 22.17, 14.14.

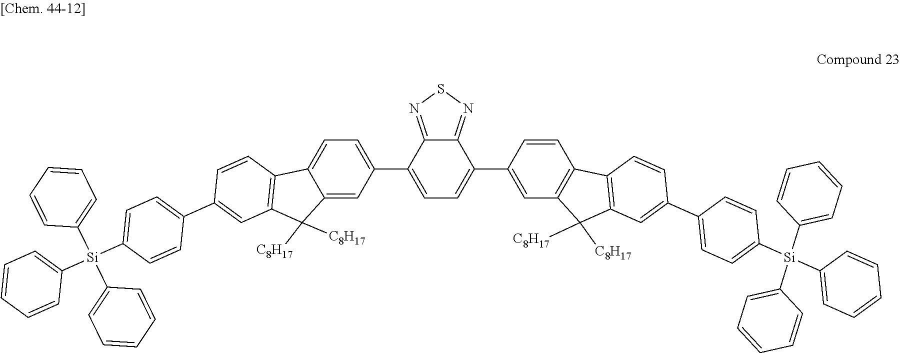

¹H NMR (CDCl₃, 300 MHz, ppm) δ: 8.06 (dd, J=10 Hz, 2H), 7.98 (s, 2H), 7.91-7.89 (m, 4H), 7.84 (d, J=8 Hz, 2H), 7.75-7.771 (m, 6H), 7.66-7.62 (m, 16H), 7.47-7.38 (m, 18H), 2.12-2.05 (m, 8H), 1.19-1.02 (m, 48H), 0.81-0.76 (t, 12H); 13C NMR (CDCl₃, 75 MHz, ppm) δ: 154.49, 152.18, 151.58, 142.76, 141.08, 140.38, 140.08, 137.08, 136.55, 136.42, 134.37, 133.68, 132.99, 129.78, 128.41, 128.05, 126.75, 126.30, 124.07, 121.73, 120.45, 120.00, 55.49, 40.48, 31.94, 30.20, 29.38, 29.35, 24.08, 22.74, 14.21.

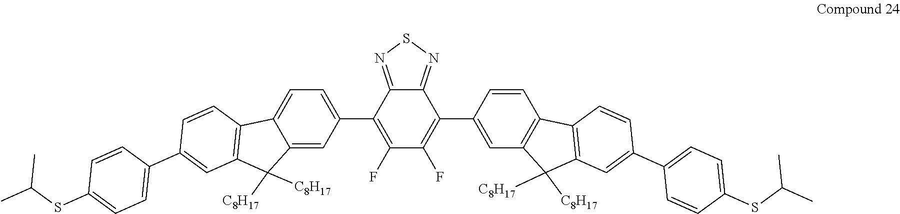

¹H NMR (CDCl₃, 300 MHz, ppm) δ: 7.97-7.84 (m, 8H), 7.69-7.61 (m, 8H), 7.53 (d, ³J=8.4 Hz, 4H), 3.47 (m, 2H), 2.10 (m, 8H), 1.39 (s, 6H), 1.37 (s, 6H), 1.25-1.11 (m, 40H), 0.93-0.77 (m, 20H); ¹⁹F NMR (CDCl₃, 282 MHz, ppm) δ: −133.19 (s, 2F); ¹³C NMR (CDCl₃, 75 MHz, ppm) δ: 152.27, 152.03, 151.06, 150.87, 150.82, 150.77, 148.88, 148.61, 141.64, 139.98, 139.92, 139.88, 134.64, 132.26, 129.49, 128.88, 127.55, 126.05, 125.57, 121.39, 120.51, 119.80, 119.23, 119.15, 119.11, 119.03, 55.46, 40.30, 38.36, 31.85, 30.09, 29.27, 29.25, 23.97, 23.22, 22.65, 14.12.

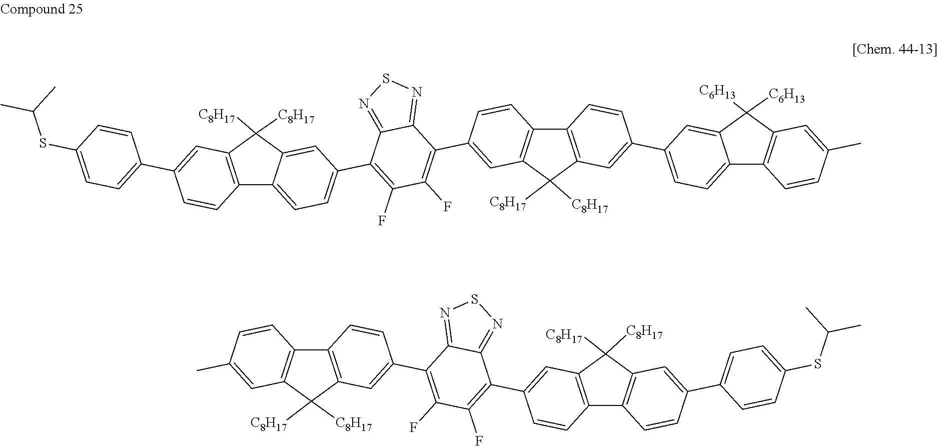

¹H NMR (CDCl₃, 300 MHz, ppm) δ: 8.00-7.83 (m, 18H), 7.77-7.68 (m, 8H), 7.67-7.61 (m, 8H), 7.52 (d, ³J=8.4 Hz, 4H), 3.46 (m, 2H), 2.12 (m, 20H), 1.38 (s, 6H), 1.36 (s, 6H), 1.25-1.09 (m, 92H), 0.99-0.77 (m, 50H); ¹⁹F NMR (CDCl₃, 282 MHz, ppm) δ: −133.24 (s, 2F); ¹³C NMR (CDCl₃, 75 MHz, ppm) δ: 152.25, 152.20, 152.00, 151.97, 151.82, 151.05, 151.01, 150.82, 150.74, 141.68, 141.57, 141.10, 140.41, 140.10, 139.94, 139.85, 139.82, 139.63, 134.54, 132.21, 129.40, 128.82, 128.72, 127.49, 126.28, 125.98, 125.53, 121.53, 121.34, 120.43, 120.39, 120.01, 119.72, 119.06, 55.41, 55.39, 40.22, 38.32, 31.79, 31.46, 30.02, 29.66, 29.21, 23.97, 23.89, 23.15, 22.58, 22.55, 14.04.

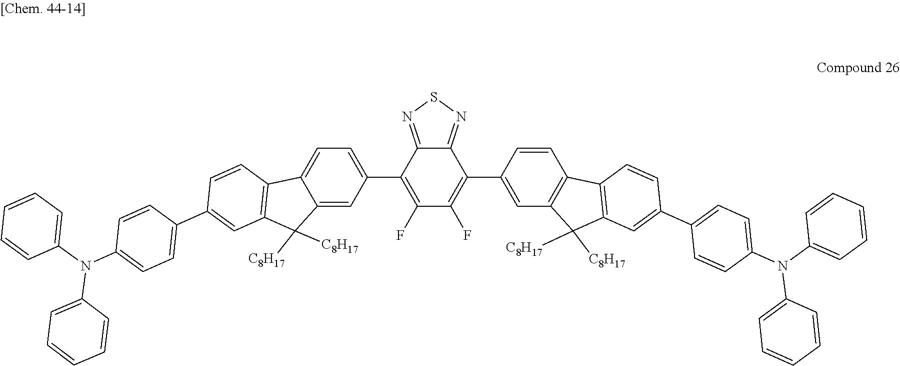

¹H NMR (CD₂Cl₂, 300 MHz, ppm) δ: 8.00-7.88 (m, 8H), 7.72-7.64 (m, 8H), 7.39-7.31 (m, 8H), 7.25-7.17 (m, 12H), 7.14-7.08 (m, 4H), 2.20-2.10 (m, 8H), 1.30-1.15 (m, 40H), 0.95-0.80 (m, 20H); ¹⁹F NMR (CD₂Cl₂, 282 MHz, ppm) δ: −134.25 (s, 2F); ¹³C NMR (CD₂Cl₂, 75 MHz, ppm) δ: 152.80, 152.49, 151.57, 151.41, 151.36, 149.36, 149.09, 148.29, 147.84, 142.28, 140.67, 139.91, 135.95, 130.11, 129.88, 129.37, 128.36, 126.16, 124.99, 124.48, 123.58, 121.69, 121.01, 120.07, 119.73, 119.66, 119.61, 119.54, 55.99, 40.76, 35.22, 32.39, 30.60, 29.80, 25.83, 24.55, 23.19, 14.42.

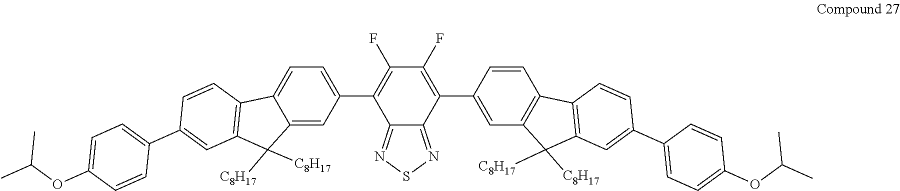

¹H NMR (CDCl₃, 300 MHz, ppm) δ: 7.85 (m, 8H), 7.61 (m, 8H); 7.02 (d, ³J=8.7 Hz, 4H), 4.64 (m, 2H), 2.07 (m, 8H), 1.41 (s, 6H), 1.39 (s, 6H), 1.12 (m, 40H), 0.83 (m, 20H); ¹⁹F NMR (CDCl₃, 282 MHz, ppm) δ: −133.29 (s, 2F); ¹³C NMR (CDCl₃, 75 MHz, ppm) δ: 157.50, 152.13, 150.96, 150.82, 148.84, 141.81, 140.35, 139.11, 133.94, 129.37, 128.61, 128.26, 125.70, 125.49, 121.17, 120.35, 119.60, 119.09, 116.18,

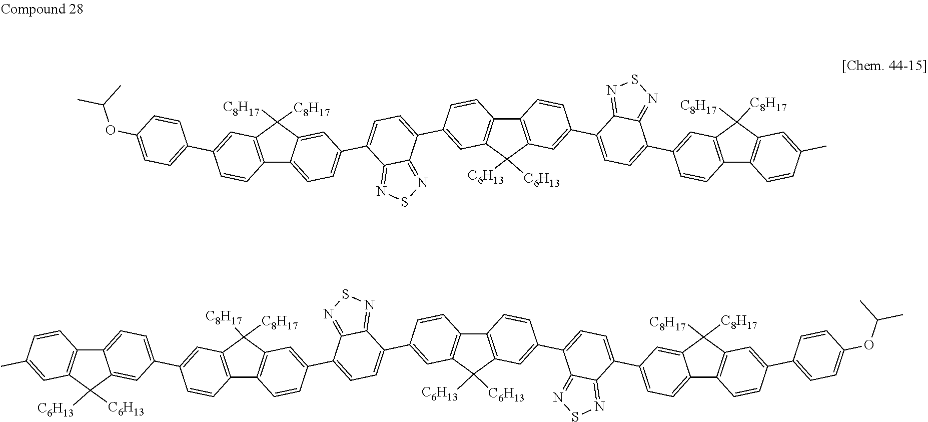

¹H NMR (CDCl₃, 300 MHz, ppm) δ: 8.10 (m, 14H), 7.89 (m, 24H), 7.75 (m, 8H2.15 (m, 28)), 7.62 (m, 8H), 7.03 (d, ³J=8.7 Hz, 4H), 4.64 (m, 2H), 2.14 (m, 28H), 1.42 (s, 6H), 1.40 (s, 6H), 1.76 (m, 128H), 0.90 (m, 84H); ¹³C NMR (CDCl₃, 75 MHz, ppm) δ: 157.48, 154.43, 152.20, 152.04, 151.87, 151.81, 151.50, 151.38, 141.21, 141.10, 140.93, 141.90, 141.87, 140.54, 140.12, 140.08, 139.92, 139.89, 139.35, 136.55, 136.53, 136.23, 136.04, 134.03, 133.74, 133.71, 133.61, 133.54, 128.36, 128.33, 128.26, 128.02, 127.96, 127.93, 126.28, 125.69, 124.10, 123.96, 121.59, 121.21, 121.18, 120.29, 120.26, 120.24, 120.10, 119.90, 119.87, 119.85, 119.73, 116.19, 70.02, 55.47, 55.44, 55.41, 55.35, 40.40, 34.71, 31.87, 31.63, 31.58, 31.52, 30.13, 29.86, 29.73, 29.30, 29.27, 25.32, 24.08, 23.97, 22.70, 22.66, 22.62, 22.15, 20.74, 14.17, 14.12.

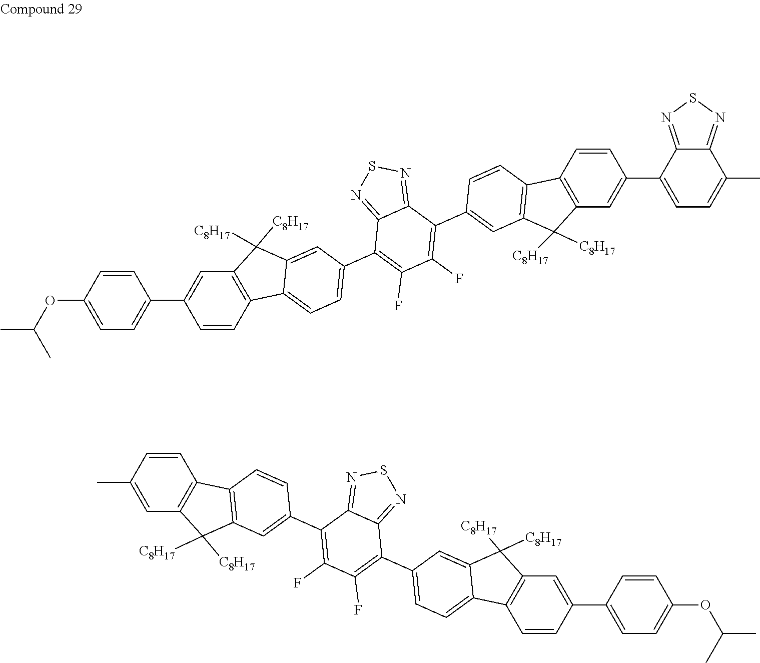

¹H NMR (CDCl₃, 300 MHz, ppm) δ: 8.10 (m, 4H), 7.92 (m, 18H), 7.62 (m, 8H), 7.03 (d, ³J=8.7 Hz, 4H), 4.64 (m, 2H), 2.13 (m, 16H), 1.42 (s, 6H), 1.40 (s, 6H), 1.16 (m, 80H), 0.84 (m, 40H); ¹⁹F NMR (CDCl₃, 282 MHz, ppm) δ: −133.16 (d, ³J=9.6 Hz, 2F), −133.26 (d, ³J=10.1 Hz, 2F); ¹³C NMR (CDCl₃, 75 MHz, ppm) δ: 157.52, 154.39, 152.15, 151.94, 151.41, 150.99, 150.89, 150.86, 150.80, 150.76, 141.86, 141.55, 140.72, 140.38, 139.12, 136.78, 133.94, 133.64, 129.49, 129.42, 129.12, 128.60, 128.41, 128.27, 128.07, 126.08, 126.05, 125.72, 125.68, 125.64, 125.54, 125.51, 124.09, 121.19, 120.38, 120.25, 120.02, 119.64, 119.36, 119.33, 119.18, 119.16, 119.12, 119.10, 118.96, 118.93, 116.19, 70.02, 55.52, 55.38, 40.31, 40.19, 31.87, 31.85, 29.30, 29.28, 29.24, 24.09, 23.96, 22.65, 22.64, 22.14, 14.10.

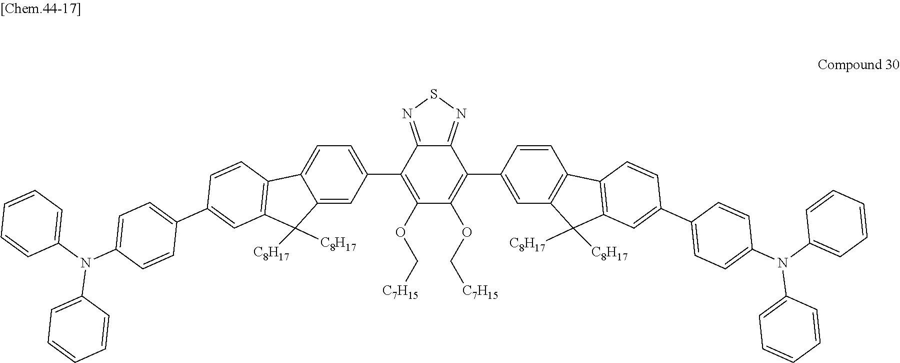

¹H NMR (CD₂Cl₂, 300 MHz, ppm) δ: 7.93-7.87 (m, 4H), 7.79-7.70 (m, 4H), 7.68-7.64 (m, 8H), 7.37-7.31 (m, 8H), 7.23-7.17 (m, 12H), 7.12-7.08 (m, 4H), 3.93 (t, J=6.7 Hz, 4H), 2.15-2.10 (m, 8H), 1.66-1.61 (m, 4H), 1.24-1.16 (m, 62H), 0.90-0.82 (m, 26H); ¹³C NMR (CD₂Cl₂, 75 MHz, ppm) δ: 153.71, 153.05, 152.59, 151.07, 148.30, 147.72, 141.03, 140.40, 140.16, 136.14, 133.32, 130.37, 129.85, 128.32, 126.27, 126.03, 125.33, 124.94, 124.53, 123.53, 121.63, 120.67, 119.62, 75.05, 55.84, 40.87, 32.42, 30.85, 30.71, 30.00, 29.92, 29.88, 26.58, 24.64, 23.25, 23.19, 14.42.

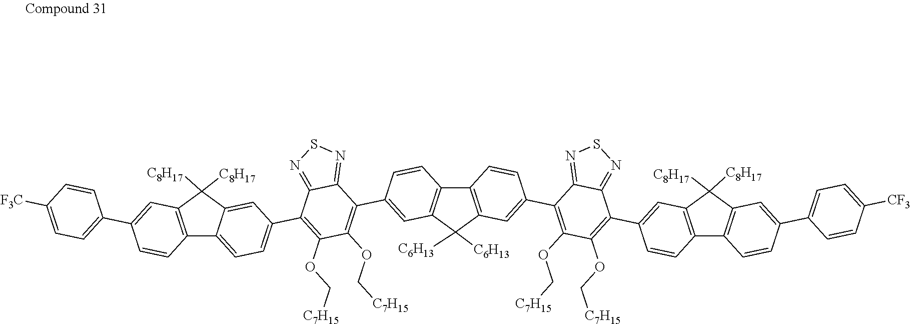

¹H NMR (CDCl₃, 300 MHz, ppm) δ: 7.97-8.84 (m, 6H), 7.83-7.36 (m, 12H), 7.73 (d, &; ³J=8.7 Hz, 4H), 7.65-7.60 (m, 4H), 4.00 (t, ³J=6.6 Hz, 8H), 2.09 (m, 12H), 1.61 (m, 8H), 1.34-1.06 (m, 92H), 1.02-0.68 (m, 42H); ¹⁹F NMR (CDCl₃, 282 MHz, ppm) δ: −62.27 (s, 6F); ¹³C NMR (CDCls, 75 MHz, ppm) δ: 153.14, 152.53, 152.48, 152.21, 150.90, 150.69, 145.29, 141.22, 140.71, 140.17, 138.62, 133.11, 132.78, 129.74, 129.63, 129.36, 128.96, 127.48, 126.32, 126.22, 125.75, 125.70, 124.92, 124.69, 122.62, 121.70, 120.34, 119.58, 119.51, 74.60, 55.41, 55.32, 40.36, 31.97, 31.87, 30.35, 30.18, 30.02, 29.46, 29.36, 29.34, 26.04, 24.22, 24.07, 22.74, 22.70, 22.64, 14.11, 14.08,

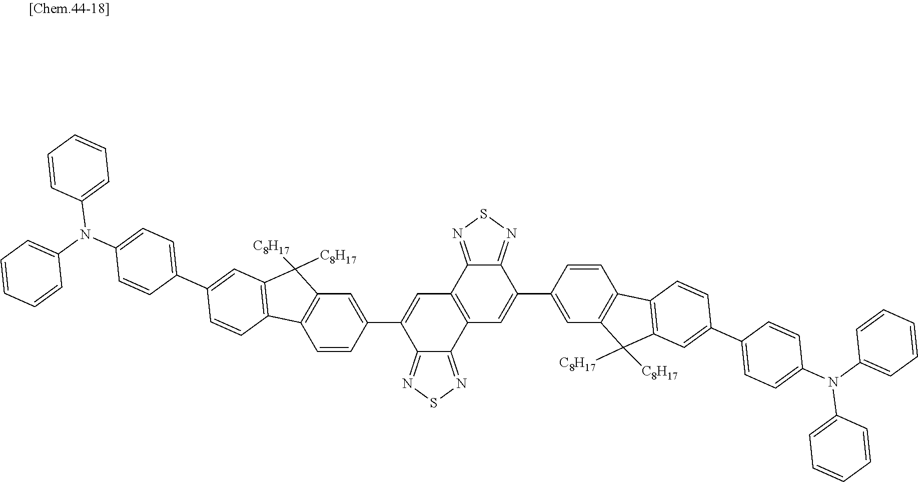

¹H NMR (CD₂Cl₂, 300 MHz, ppm) δ: 9.13 (s, 2H), 8.20-8.17 (m, 2H), 7.94 (d, J=7.9 Hz, 2H), 7.86 (d, J=8.4 Hz, 2H), 7.66-7.61 (m, 8H) 7.33-7.28 (m, 8H), 7.19-7.13 (m, 12H), 7.09-7.04 (m, 4H), 2.20-2.11 (m, 8H), 1.19-1.13 (m, 40H), 0.79-0.75 (m, 12H); ¹³C NMR (CDCl₃, 75 MHz, ppm) δ: 154.69, 154.62, 152.77, 152.02, 148.28, 147.79, 142.11, 140.47, 140.06, 136.30, 135.99, 134.69, 129.96, 129.18, 128.33, 126.10, 125.85, 125.46, 124.96, 124.80, 124.48, 123.55, 121.68, 120.91, 120.28, 56.04, 40.84, 32.37, 30.60, 29.90, 24.57, 23.17, 14.39.

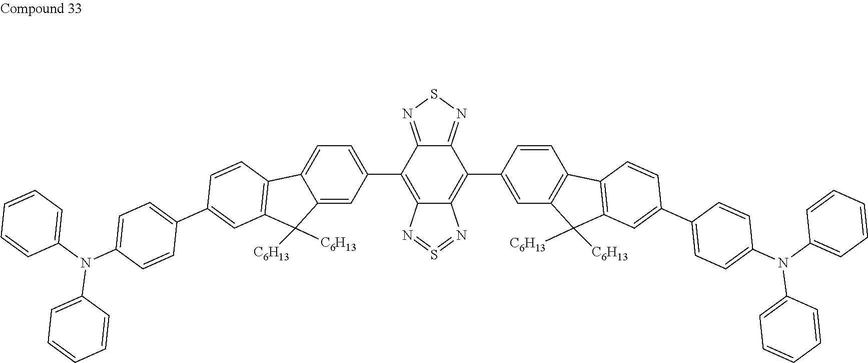

¹H NMR (CD₂Cl₂, 300 MHz, ppm) δ: 8.38 (d, ⁴J=0.9 Hz, 2H), 8.32 (dd, ³J=8.0 Hz, ⁴J=1.3 Hz, 2H), 7.98 (d, ³J=7.8 Hz, 2H), 7.89 (d, ³J=7.8 Hz, 2H), 7.70-7.61 (m, 8H), 7.35-7.27 (m, 8H), 7.23-7.13 (m, 12H), 7.11-7.02 (m, 4H), 2.22-2.09 (m, 8H), 1.24-1.16 (m, 24H), 0.97-0.92 (m, 8H), 0.81 (t, ³J=6.7 Hz, 12H); ¹³C NMR (CD₂Cl₂, 75 MHz, ppm) δ: 153.58, 153.13, 151.33, 148.27, 147.80, 142.00, 140.57, 140.03, 136.01, 134.50, 131.67, 129.87, 128.37, 127.84, 126.17, 124.96, 124.50, 123.56, 122.13, 121.71, 121.06, 119.85, 55.96, 40.79, 32.15, 30.36, 24.65, 23.20, 14.44.

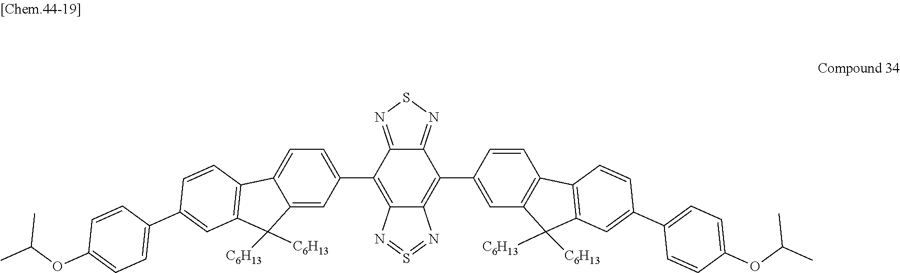

¹H NMR (CDCl₃, 300 MHz, ppm) δ: 8.34-8.30 (m, 4H), 8.00 (d, ³J=8.7 Hz, 2H), 7.87 (d, ³J=8.4 Hz, 2H), 7.68-7.60 (m, 8H), 7.04 (d, ³J=8.7 Hz, 4H), 4.66 (m, 2H), 2.20-2.05 (m, 8H), 1.43 (s, 6H), 1.41 (s, 6H), 1.23-681.15 (m, 24H), 0.99-0.02 (m, 8H), 0.81 (t, ³J=6.8 Hz, 12H); ¹³C NMR (CDCl₃, 75 MHz, &ppm) δ: 157.60, 153.16, 152.62, 150.94, 141.78, 140.39, 139.37, 134.13, 133.79, 130.91, 128.39, 127.36, 125.84, 121.90, 121.33, 120.53, 119.59, 116.31, 70.15, 55.47, 40.42, 31.69, 29.96, 24.15, 22.78, 22.26, 14.22.

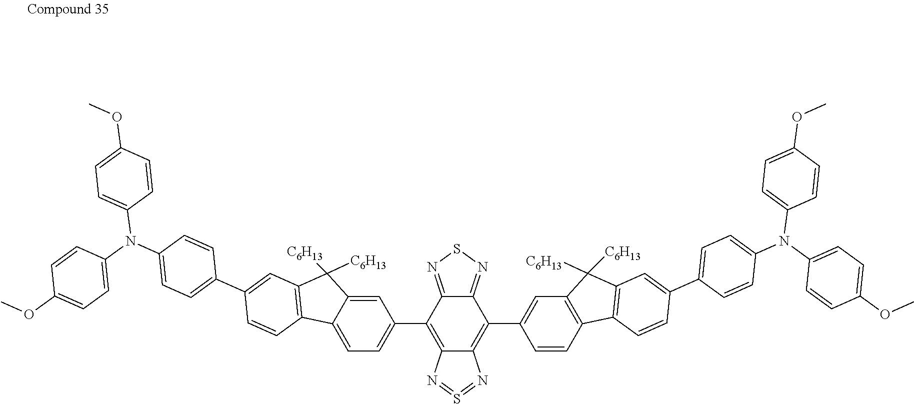

¹H NMR (CD₂Cl₂, 300 MHz, ppm) δ: 8.40 (d, ⁴J=0.9 Hz, 2H), 8.35 (dd, ³J=8.1 Hz, ⁴J=1.5 Hz, 2H), 8.01 (d, ³J=8.1 Hz, 2H), 7.91 (d, ³J=7.8 Hz, 2H), 7.72-7.64 (m, 4H), 7.61 (d, ³J=8.7 Hz, 4H), 7.16 (d, ³J=8.7 Hz, 8H), 7.07 (d, ³J=8.7 Hz, 4H), 6.93 (d, ³J=9.0 Hz, 8H), 3.85 (s, 12H), 2.18 (m, 8H), 1.30-1.15 (m, 24H), 1.05-0.91 (m, 8H), 0.84 (t, ³J=6.6 Hz, 12H); ¹³C NMR (CD₂Cl₂, 75 MHz, ppm) δ: 156.11, 153.01, 152.53, 150.74, 148.28, 141.51, 140.83, 140.22,

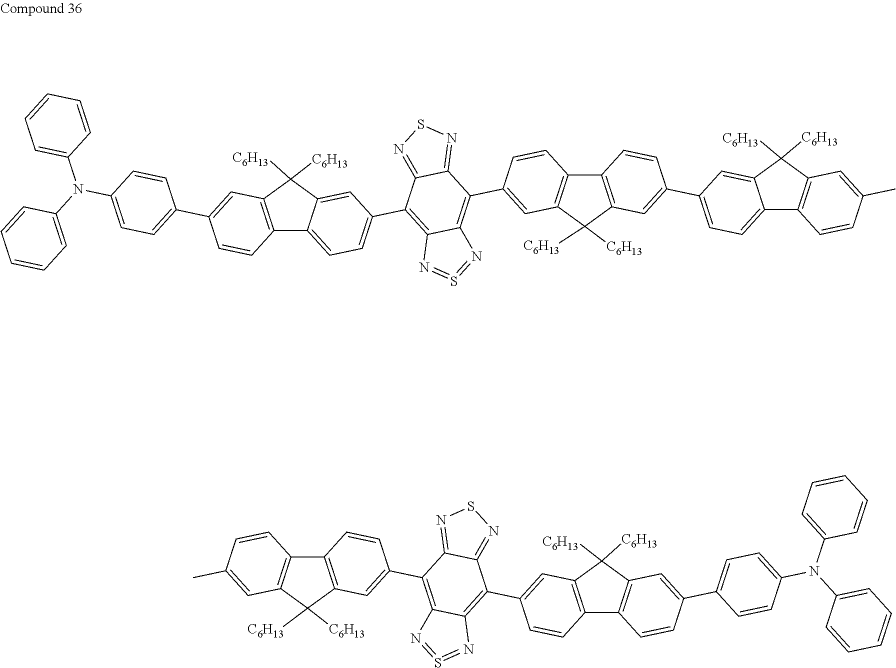

¹H NMR (CD₂Cl₂, 300 MHz, ppm) δ: 8.32-8.22 (m, 8H), 7.97-7.86 (m, 6H), 7.82 (d, ³J=8.4 Hz, 4H), 7.73-7.66 (m, 8H), 7.60-7.52 (m, 8H), 7.26-7.19 (m, 8H), 7.13-7.01 (m, 12H), 7.02-6.95 (m, 4H), 2.20-1.96 (m, 20H), 1.18-1.03 (m, 60H), 0.95-0.67 (50H); ¹³C NMR (CD₂Cl₂, 75 MHz, ppm) δ: 153.60, 153.20, 153.13, 152.47, 151.41, 151.35, 148.28, 147.82, 142.01, 141.96, 141.60, 141.07, 140.73, 140.58, 140.39, 140.03, 138.50, 136.61, 136.50, 136.01, 134.60, 134.49, 131.65, 131.54, 129.87, 128.35, 127.89, 127.84, 126.82, 126.75, 126.16, 124.97, 124.48, 123.57, 122.24, 122.19, 122.13, 121.72, 121.04, 120.60, 119.94, 119.84, 56.03, 55.96, 40.96, 40.77, 32.15, 32.10, 30.34, 30.25, 24.65, 23.19, 23.15, 14.41, 14.38.

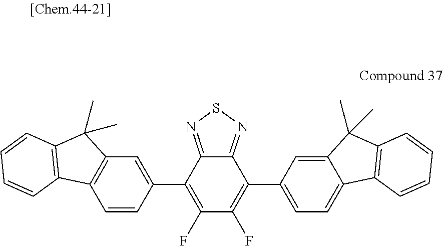 [SMILES: CC1(C)c2ccccc2-c2ccc(-c3c(F)c(F)c(-c4ccc5c(c4)C(C)(C)c4ccccc4-5)c4nsnc34)cc21]

¹H NMR (CDCl₃, 500 MHz, ppm) δ: 7.93 (m, 4H), 7.87 (d, J=7.9 Hz, 2H), 7.82 (d, J=6.6 Hz, 2H), 7.50 (d, J=7.0 Hz, 2H), 7.38 (m, 4H), 1.58 (s, 12H)

¹⁹F NMR (CDCl₃, 471 MHz, ppm) δ: −133.19 (s)

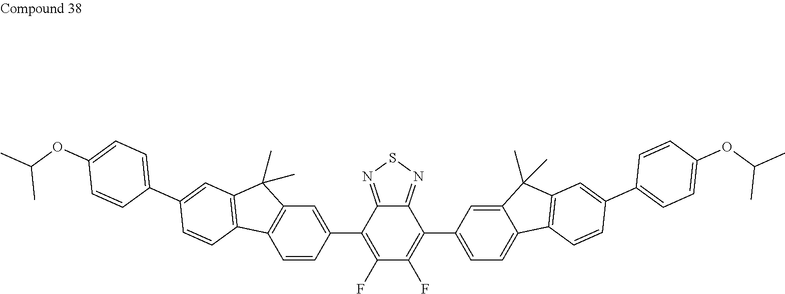 [SMILES: CC(C)Oc1ccc(-c2ccc3c(c2)C(C)(C)c2cc(-c4c(F)c(F)c(-c5ccc6c(c5)C(C)(C)c5cc(-c7ccc(OC(C)C)cc7)ccc5-6)c5nsnc45)ccc2-3)cc1]

¹H NMR (CDCl₃, 500 MHz, ppm) δ: 7.93 (m, 4H), 7.88 (d, J=8.1 Hz, 2H), 7.84 (d, J=7.9 Hz, 2H), 7.65 (s, 2H), 7.60 (m, 6H), 7.00 (d, J=8.3 Hz, 4H), 4.63 (m, 2H), 1.62 (s, 12H), 1.40 (d, J=6.0 Hz, 12H)

¹⁹F NMR (CDCl₃, 471 MHz, ppm) δ: −133.20 (s)

¹³C NMR (CDCl₃, 126 MHz, ppm) δ: 157.69, 154.94, 154.03, 140.86, 140.19, 137.36, 133.98, 129.81, 128.41, 126.07, 125.18, 121.18, 120.83, 120.08, 116.34, 77.41, 77.16, 76.91, 70.18, 47.33, 31.74, 27.39, 22.80, 22.26, 14.26.

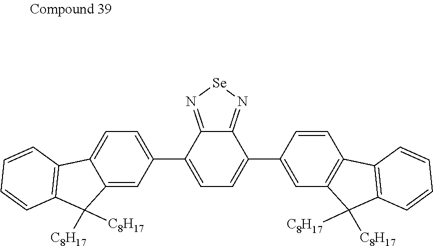

¹H NMR (CDCl₃, 300 MHz, ppm) δ: 7.97 (dd, J=7.8 Hz, J=1.5 Hz, 2H), 7.88-7.83 (m, 4H), 7.79-7.75 (m, 2H), 7.72 (s, 2H), 7.42-7.29 (m, 6H), 2.13-1.94 (m, 8H), 1.27-1.03 (m, 40H), 0.84-0.70 (m, 20H).

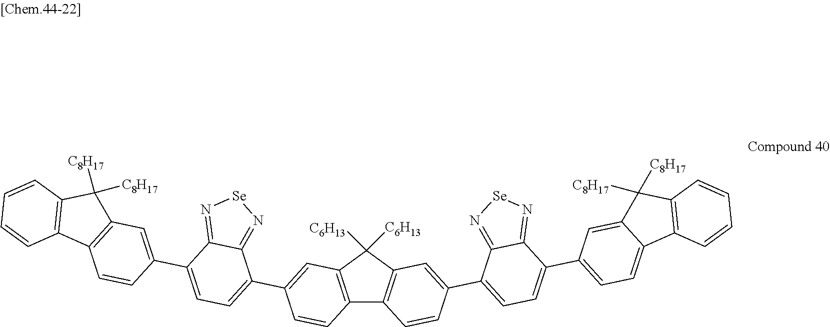

¹H NMR (CDCl₃, 300 MHz, ppm) δ: 8.03-7.97 (m, 4H), 7.95-7.92 (m, 2H), 7.90-7.85 (m, 6H), 7.79-7.75 (m, 6H), 2.16-2.02 (m, 12H), 1.21-1.10 (m, 56H), 0.83-0.77 (m, 26H)

¹³C NMR (CDCl₃, 75 MHz, ppm) δ: 160.17, 151.77, 151.45, 151.15, 141.37, 140.93, 140.86, 137.21, 136.94, 135.62, 135.50, 128.76, 128.62, 128.51, 128.46, 127.36, 126.98, 124.36, 124.25, 124.20, 123.10, 120.07, 119.97, 119.67, 55.51, 55.34, 40.42, 34.81, 31.97, 31.74, 31.69, 30.24, 29.97, 29.41, 29.39, 25.43, 24.21, 24.05, 22.78, 22.76, 14.23.

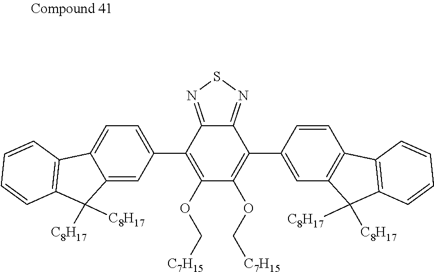

¹H NMR (CDCl₃, 300 MHz, ppm) δ: 7.88 (d, 2H, J=7.8 Hz), 7.79-7.77 (m, 3H), 7.75-7.74 (m, 3H), 7.41-7.31 (m, 6H), 3.91 (t, 3H, J=6.76 Hz), 2.08-1.97 (m, 8H,), 1.61-1.57 (m, 4H), 1.20-1.10 (m, 66H), 0.86-0.80 (m, 26H).

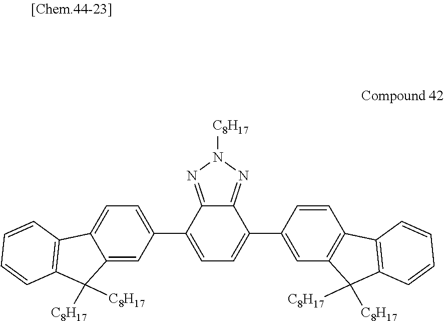

¹H NMR (CDCl₃, 300 MHz, ppm) δ: 8.17-8.14 (dd, 2H, J=7.92 Hz, J=1.51 Hz), 8.08 (d, 2H, J=1.14 Hz), 7.87 (d, 2H, J=7.93 Hz), 7.80-7.7- (m, 4H), 7.42-7.31 (m, 6H), 4.84 (t, 2h, J=7.2 Hz), 2.27-2.22 (m, 2H), 2.11-2.02 (m, 8H), 1.49-1.31 (m, 12H), 1.20-1.11 (m, 36H), 0.84-0.80 (m, 25H)

¹³C NMR (CDCl₃, 75 MHz, ppm) δ: 151.45, 151.21, 143.84, 141.06, 140.99, 136.34, 130.70, 127.61, 127.20, 126.93, 124.48, 123.31, 123.07, 119.98, 119.95, 56.94, 55.27, 40.52, 31.95, 30.31, 30.21, 29.44, 29.40, 29.36, 29.29, 26.90, 24.06, 22.80, 22.75, 14.24, 14.21.

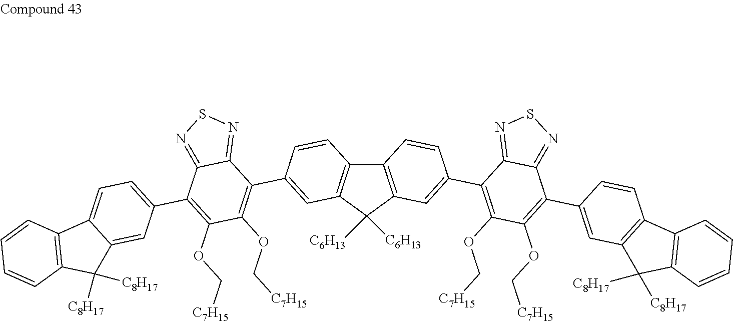

¹H NMR (CDCl₃, 300 MHz, ppm) δ: 7.94 (d, 2H, J=8.02 Hz), 7.88 (d, 2H, J=7.76 Hz), 7.81-7.74 (m, 10H), 7.40-7.31 (m, 6H), 3.94-3.89 (m, 8H), 2.10-2.00 (m, 12H), 1.60-1.56 (m, 8H), 1.22-1.09 (m, 92H), 0.88-0.76 (m, 42H).

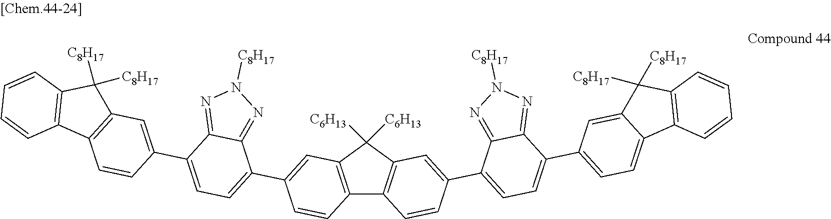

¹H NMR (CDCl₃, 300 MHz, ppm) δ: 8.20 (t, 4H, J=7.75 Hz), 8.14 (m, 4H), 7.95 (d, 2H, J=7.93 Hz), 7.90 (d, 2H, J=7.93 Hz), 7.80 (m, 6H), 7.44-7.33 (m, 6H), 4.88 (t, 4H, J=7.14 Hz), 2.30-2.05 (m, 16H), 1.52-1.34 (m, 28H), 1.18-1.14 (m, 56H), 0.95-0.79 (m, 24H). 13C NMR (CDCl₃, 75 MHz, ppm) δ: 151.85, 151.45, 151.22, 143.86, 141.07, 140.99, 140.79, 136.46, 136.34, 130.74, 130.71, 127.75, 127.63, 127.21, 126.94, 124.51, 123.40, 123.32, 123.07, 120.18, 119.98, 56.97, 55.39, 55.27, 40.53, 31.95, 31.70, 30.31, 30.23, 30.09, 29.44, 29.41, 29.36, 29.29, 26.90, 24.22, 24.06, 22.83, 22.80, 22.75, 14.24, 14.21, 14.16.

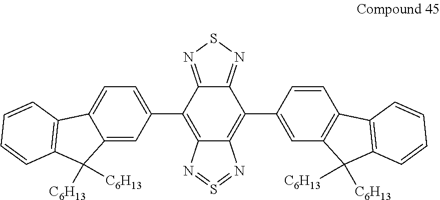

¹H NMR (CDCl₃, 300 MHz, ppm) δ: 8.42 (d, 2H, J=1.1 Hz), 8.37 (dd, 2H, J=7.92 Hz, J=1.47 Hz), 8.04 (d, 2H, J=7.94 Hz), 7.90-7.87 (m, 2H), 7.52-7.43 (m, 6H), 2.24-2.11 (m, 8H), 1.26-1.23 (m, 26H), 1.04-0.84 (m, 22H).

¹³C NMR (CDCl₃, 75 MHz, ppm) δ: 153.08, 151.85, 150.62, 141.91, 140.67, 133.87, 130.88, 127.59, 127.31, 127.01, 123.07, 121.84, 120.28, 119.58, 55.34, 40.33, 31.65, 29.91, 24.07, 22.71, 14.20.

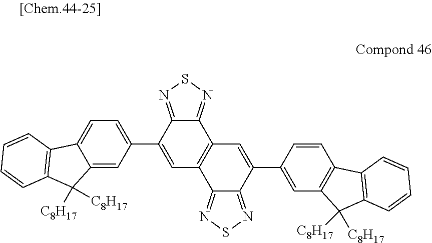

¹H NMR (CDCl₃, 300 MHz, ppm) δ: 9.09 (s, 2H), 8.20 (d, 2H, J=1.1 Hz), 8.16 (dd, 2H, J=7.9 Hz, J=1.51 Hz), 7.92 (d, 2H, J=7.9 Hz), 7.84-7.81 (m, 2H), 7.47-7.37 (m, 6H), 2.19-2.09 (m, 8H), 1.23-1.15 (m, 40H), 0.84-0.79 (m, 20H).

¹³C NMR (CDCl₃, 75 MHz, ppm) δ: 154.17, 154.06, 151.55, 151.34, 141.99, 140.72, 135.72, 134.26, 128.58, 127.57, 127.02, 125.30, 125.00, 124.28, 123.14, 120.22, 119.97, 55.48, 40.45, 31.95, 30.23, 29.40, 24.09, 22.74, 14.21.

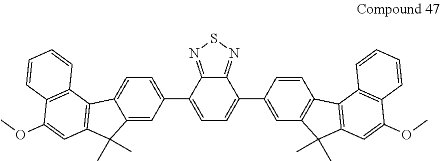

¹H NMR (CDCl₃, 300 MHz, ppm) δ: 8.75 (d, 2H, J=8.42 Hz), 8.41 (dd, 4H, J=8.62 Hz, J=2.45 Hz), 8.14-8.18 (m, 4H), 7.95 (s, 2H), 7.70 (t, 2H, J=7.06 Hz), 7.55 (t, 2H, J=7.39 Hz), 7.00 (s, 2H), 4.14 (s, 6H), 1.66 (s, 12H).

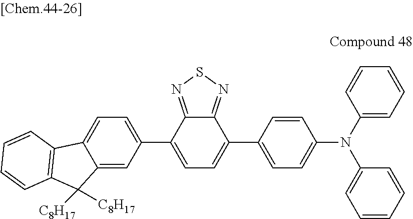

¹H NMR (CD₂Cl₂, 300 MHz, ppm) δ: 8.04 (m, 2H), 7.96 (d, 2H, J=Hz), 7.90-7.86 (m, 2H), 7.82-7.79 (m, 2H), 7.46-7.31 (m, 7H), 7.25-7.20 (m, 6H), 7.13-7.08 (m, 2H), 2.14-2.05 (m, 4H), 1.23-1.12 (m, 20H), 0.85-0.80 (m, 10H).

¹³C NMR (CD₂Cl₂, 75 MHz, ppm) δ: 154.93, 154.75, 151.92, 151.63, 148.63, 148.11, 141.82, 141.26, 136.85, 133.60, 132.96, 131.64, 130.63, 129.96, 128.79, 128.55, 127.89, 127.80, 127.45, 125.43, 124.54, 123.93, 123.62, 123.37, 120.49, 120.16, 55.83, 40.83, 32.40, 30.64, 29.84, 29.82, 24.53, 23.20, 14.46.

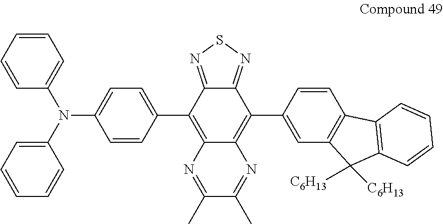

¹H NMR (CD₂Cl₂, 300 MHz, ppm) δ: 7.92-7.88 (m, 3H), 7.86-7.83 (m, 3H), 7.46-7.43 (m, 1H), 7.41-7.33 (m, 6H), 7.28-7.22 (m, 6H), 7.15-7.09 (m, 2H), 2.73 (s, 3H), 2.68 (s, 3H), 2.09-2.02 (m, 4H), 1.17-1.12 (m, 12H), 0.80-8.75 (m, 10H).

¹³C NMR (CD₂Cl₂, 75 MHz, ppm) δ: 155.59, 155.46, 153.40, 153.25, 152.07, 150.19, 148.31, 148.11, 141.42, 141.37, 137.67, 137.47, 134.44, 134.30, 131.77, 129.98, 129.77, 129.14, 128.82, 127.78, 127.41, 125.77, 124.06, 123.61, 121.76, 120.50, 119.29, 55.64, 40.87, 32.17, 30.45, 24.63, 24.12, 23.22, 14.34.

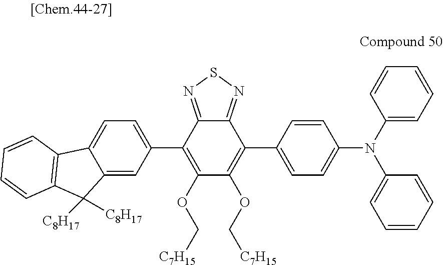

¹H NMR (CD₂Cl₂, 300 MHz, ppm) δ: 7.85 (d, 1H, J=7.4 Hz), 7.81-7.78 (m, 1H), 7.33-7.70 (m, 2H), 7.64 (d, 2H, J=8.8 Hz), 7.43-7.29 (m, 7H), 7.22-7.18 (m, 6H), 7.11-7.06 (m, 2H), 3.98 (t, 2H, J=6.5 Hz), 3.90 (t, 2H, J=6.7 Hz), 2.06-2.0 (m, 4H), 1.64-1.56 (m, 4H), 1.30-1.10 (m, 40H), 0.91-0.75 (m, 16H).

¹³C NMR (CD₂Cl₂, 75 MHz, ppm) δ: 153.76, 153.70, 153.10, 152.84, 151.82, 150.80, 148.27, 148.03, 141.47, 141.29, 133.34, 132.33, 130.25, 129.89, 128.35, 127.71, 127.36, 126.22, 125.28, 125.19, 124.57, 123.72, 123.56, 122.95, 120.35, 119.58, 74.99, 55.71, 40.82, 32.49, 32.42, 32.40, 30.82, 30.78, 30.72, 30.03, 29.96, 29.94, 29.90, 29.87, 29.85, 26.61, 26.56, 24.59, 23.28, 23.24, 23.19, 14.48, 14.42.

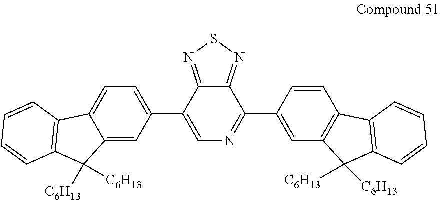

¹H NMR (CDCl₃, 300 MHz, ppm) δ: 8.97 (s, 1H), 8.77 (dd, 1H, J=8.0 Hz, J=1.5 Hz), 8.69 (d, 1H, J=1.2 Hz), 8.09 (m, 1H), 8.03 (d, 1H, J=1.2 Hz), 7.94-7.89 (m, 2H), 7.83-7.78 (m, 2H), 7.42-7.36 (m, 6H), 2.16-2.02 (m, 8H), 1.21-1.08 (m, 22H), 0.83-0.77 (m, 12H).

¹³C NMR (CDCl₃, 75 MHz, ppm) δ: 157.13, 152.47, 151.92, 151.53, 151.48, 151.21, 150.21, 143.59, 142.81, 141.98, 140.62, 135.83, 133.45, 129.34, 128.20, 127.88, 127.61, 127.36, 127.04, 124.47, 123.87, 123.17, 123.14, 120.49, 120.20, 120.15, 119.86, 55.51, 55.40, 40.44, 31.94, 30.21, 29.37, 24.01, 22.74, 22.72, 14.21.

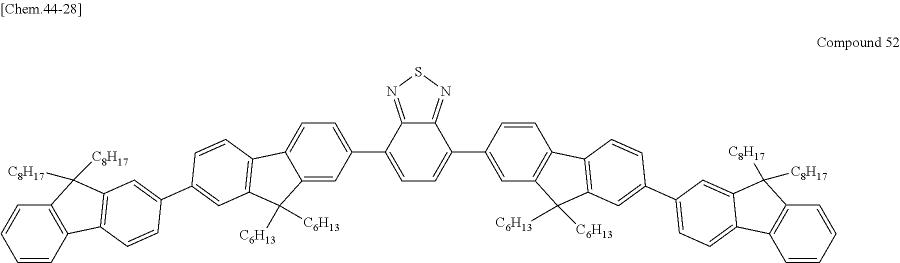

¹H NMR (CDCl₃, 300 MHz, ppm) δ: 8.08 (d, 2H, J=7.9 Hz), 8.01 (s, 2H), 7.93 (m, 4H), 7.86 (d, 2H, J=7.8 Hz), 7.80 (d, 2H, J=7.8 Hz), 7.77-7.75 (m, 2H), 7.71-7.65 (m, 8H), 7.39-7.32 (m, 6H), 2.14-2.04 (m, 16H), 1.26-1.10 (m, 60H), 0.89-0.77 (m, 44H).

¹³C NMR (CDCl₃, 75 MHz, ppm) δ: 154.55, 152.27, 151.63, 151.57, 151.17, 141.19, 140.97, 140.93, 140.59, 140.53, 139.97, 136.32, 133.76, 128.40, 128.06, 127.12, 126.94, 126.39, 126.20, 124.16, 123.08, 121.67, 121.59, 120.33, 120.04, 119.93, 119.87, 55.52, 55.33, 40.53, 31.93, 31.64, 30.19, 29.89, 29.37, 24.09, 23.97, 22.74, 14.22, 14.20.

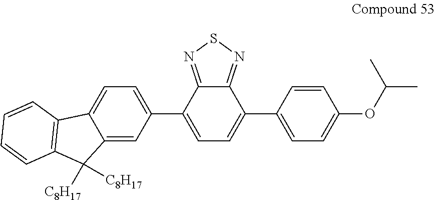

¹H NMR (CDCl₃, 300 MHz, ppm) δ: 8.06-7.95 (m, 4H), 7.90-7.85 (m, 2H), 7.82-7.77 (m, 2H), 7.45-7.37 (m, 3H), 7.10 (d, 2H, J=11.7 Hz), 4.73-4.65 (m, 2H), 2.13-2.05 (m, 4H), 1.45 (s, 3H), 1.43 (s, 3H), 1.22-1.14 (m, 20H), 0.85-0.82 (m, 10H).

¹³C NMR (CDCl₃, 75 MHz, ppm) δ: 158.33, 154.41, 154.36, 151.39, 151.16, 141.35, 140.79, 136.28, 133.21, 132.81, 130.53, 129.78, 128.22, 128.04, 127.36, 126.95, 123.95, 123.04, 120.04, 119.80, 115.95, 70.03, 55.32, 40.42, 31.92, 30.19, 29.35, 23.99, 22.71, 22.24, 14.19.

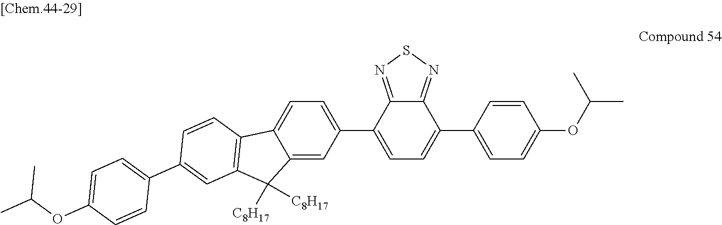

¹H NMR (CDCl₃, 300 MHz, ppm) δ: 8.04 (d, 1H, J=7.9 Hz), 7.99-7.95 (m, 3H), 7.89-7.85 (m, 2H), 7.83-7.77 (m, 2H), 7.65-7.58 (m, 4H), 7.10 (d, 2H, J=8.8 Hz), 7.04 (d, 2H, J=8.8 Hz), 4.72-4.60 (m, 2H), 2.14-2.07 (m, 4H), 1.44-1.41 (m, 12H), 1.21-1.12 (m, 2H), 0.84-0.79 (m, 10H).

¹³C NMR (CDCl₃, 75 MHz, ppm) δ: 158.34, 157.55, 154.44, 154.40, 152.09, 151.40, 141.16, 140.10, 139.46, 136.14, 134.10, 133.26, 132.81, 130.55, 129.83, 128.32, 128.04, 127.42, 125.74, 123.96, 121.24, 120.29, 119.77, 116.27, 115.98, 77.58, 77.16, 76.74, 70.08, 55.42, 40.49, 31.93, 30.19, 29.36, 29.33, 24.03, 22.72, 22.26, 22.23, 14.19.

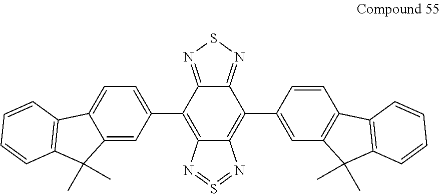 [SMILES: CC1(C)c2ccccc2-c2ccc(-c3c4c(c(-c5ccc6c(c5)C(C)(C)c5ccccc5-6)c5nsnc35)N=S=N4)cc21]

¹H NMR (CDCl₃, 300 MHz, ppm) δ: 8.38 (d, 4J=1.2 Hz, 2H), 8.29 (dd, 3J=8.1 Hz, 4J=1.5 Hz, 2H), 8.02 (dd, 3J=8.1 Hz, 2H), 7.90-7.84 (m, 2H), 7.57-7.51 (m, 2H), 7.46-7.38 (m, 4H), 1.66 (s, 12H).

¹³C NMR (CDCl₃, 75 MHz, ppm) δ: 154.50, 153.67, 153.08, 140.29, 138.93, 134.13, 131.18, 127.89, 127.28, 126.66, 122.84, 121.88, 121.51, 120.64, 120.02, 47.29, 27.34.

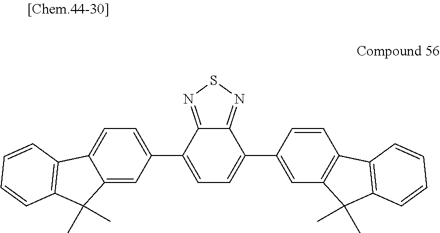 [SMILES: CC1(C)c2ccccc2-c2ccc(-c3ccc(-c4ccc5c(c4)C(C)(C)c4ccccc4-5)c4nsnc34)cc21]

¹H NMR (CDCl₃, 300 MHz, ppm) δ: 8.06-7.99 (m, 4H), 7.93-7.88 (m, 2H), 7.83-7.78 (m, 2H), 7.53-7.46 (m, 2H), 7.437.33 (m, 4H), 1.60 (s, 12H)

¹³C NMR (CDCl₃, 75 MHz, ppm) δ: 154.43, 154.24, 154.13, 139.65, 138.96, 136.60, 133.70, 128.56, 128.17, 127.66, 127.24, 123.76, 122.81, 120.44, 120.24, 47.22, 27.39.

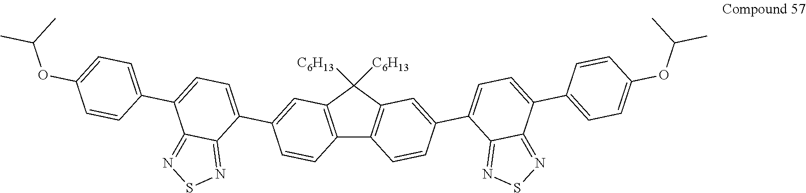

¹H NMR (CDCl₃, 300 MHz, ppm) δ: 8.05 (dd, 2H, J=Hz, J=Hz), 7.99-7.92 (m, 8H), 7.86 (d, 2H, J=Hz), 7.78 (d, 2H, J=Hz), 7.08 (d, 4H, J=Hz), 4.71-4.63 (m, 2H), 2.15-2.09 (m, 4H), 1.42 (s, 6H), 1.40 (s, 6H), 1.17-1.13 (m, 12H), 0.91 (m, 4H), 0.80-0.75 (m, 6H).

¹³C NMR (CDCl₃, 75 MHz, ppm) δ: 158.38, 154.46, 154.42, 151.84, 140.95, 136.59, 133.25, 132.96, 130.58, 129.85, 128.40, 128.17, 127.47, 124.08, 120.12, 116.02, 70.14, 55.56, 40.41, 31.65, 29.93, 24.13, 22.75, 22.27, 14.19.

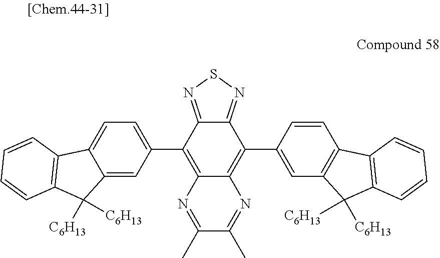

¹H NMR (CDCl₃, 300 MHz, ppm) δ: 8.06-7.99 (m, 4H), 7.94 (s, 2H), 7.87-7.85 (m, 2H), 7.48-7.39 (m, 6H), 2.74 (s, 6H), 2.10 (m, 8H), 1.20-1.16 (m, 24H), 0.83-0.79 (m, 20H).

¹³C NMR (CDCl₃, 75 MHz, ppm) δ: 154.77, 152.99, 151.57, 149.69, 141.14, 141.12, 137.23, 133.70, 131.11, 129.90, 128.41, 127.27, 126.95, 123.01, 120.11, 119.15, 55.21, 40.63, 34.78, 34.63, 31.72, 30.09, 25.39, 24.11, 23.88, 22.81, 20.82, 14.14.

### (Example 1) Thin Films

A solution of Compound 1 in chloroform was spin-coated on a precleaned fused silica substrate to form a thin neat film. In a similar way, thin films of Compounds 2 to 4, 7, 8, 10, 11, 15, 16, 23, 24, 32 and 33 were also formed.

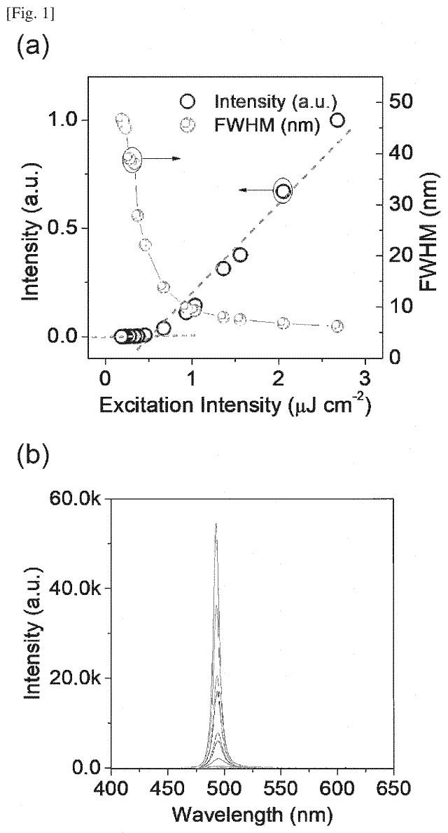

The formed thin films were used to evaluate their potential for organic lasers. The thin films were photo-excited by a pulsed nitrogen laser at 337 nm. The pulse duration of the pump laser is about 800 ps and its repetition rate is 8 Hz. The pump intensity is controlled using a set of neutral density filters. The pump beam is focused into a 0.5 cm×0.08 cm stripe. An optical fiber connected to a charge-coupled device spectrometer was used to measure the emission spectra from the edge of the organic layers. The emission spectra were measured at various pump intensity. At low excitation intensities, the PL spectra were broad and independent of the pump intensity. At high excitation intensities, ASE occurred and a spectral narrowing of the emission band was observed. The ASE threshold and a peak wavelength of ASE were determined. The results are summarized in Table 1. FIG. 1(a) shows variation of the intensity as a function of the excitation intensity and variation of the FWHM as a function of the excitation intensity for the thin film of Compound 4, and FIG. 1(b) shows variation of the spectrum as a function of the excitation intensity for the thin film.

TABLE 1

| | ^{λ}ASE | Threshold |
|---|---|---|
| Compound | [nm] | [μJ/cm²] |
| Compound 1 | 552 | 9.8 |
| Compound 2 | 554 | 4.0 |
| Compound 3 | 564 | 2.3 |
| Compound 4 | 494 | 0.50 |
| Compound 7 | 551 | 0.50 |
| Compound 8 | 544 | 1.19 |
| Compound 10 | 578 | 0.84 |
| Compound 11 | 558 | 1.56 |
| Compound 15 | 576 | 2.3 |
| Compound 16 | 554 | 0.83 |
| Compound 23 | 541 | 1.30 |
| Compound 24 | 546 | 1.14 |
| Compound 32 | 566 | 4.46 |
| Compound 33 | 815 | 4.0 |

### (Example 2) Green Optically Pumped Distributed Feedback (DFB) Organic Laser

Glass substrates were cleaned by ultrasonication using neutral detergent, pure water, acetone, and isopropanol followed by UV-ozone treatment. A 100-nm-thick layer of SiO₂, which would become the DFB grating, was sputtered at 100° C. onto glass substrates. The argon pressure during the sputtering was 0.66 Pa. The RF power was set at 100 W. Substrates were cleaned by ultrasonication using isopropanol followed by UV-ozone treatment. The SiO₂ surfaces were treated with hexamethyldisilazane (HMDS) by spin coating at 4,000 rpm for 15 s and annealed at 120° C. for 120 s. A resist layer with a thickness of around 70 nm was spin-coated on the substrates at 4,000 rpm for 30 s from a ZEP520A-7 solution (ZEON Co.) and baked at 180° C. for 240 s.

Electron beam lithography was performed to draw grating patterns on the resist layer using a JBX-5500SC system (JEOL) with an optimized dose of 0.1 nC cm-2. After the electron beam irradiation, the patterns were developed in a developer solution (ZED-N50, ZEON Co.) at room temperature. The patterned resist layer was used as an etching mask while the substrate was plasma etched with CHF₃ using an EIS-200ERT etching system (ELIONIX). To completely remove the resist layer from the substrate, the substrate was plasma-etched with 02 using a FA-lEA etching system (SAMCO). The etching conditions were optimized to completely remove the SiO₂ from the grooves in the DFB until the SiO₂ surfaces were exposed. The gratings formed on the SiO₂ surfaces were observed with SEM (SU8000, Hitachi). EDX (at 6.0 kV, SU8000, Hitachi) analysis was performed to confirm complete removal of SiO₂ from ditches in the DFB. Cross section SEM was measured by Kobelco using a cold-field-emission SEM (SU8200, Hitachi High-Technologies). The gratings composed of second-order Bragg scattering region were thus prepared onto SiO₂ over 5×5 mm² area. Grating periods (A) of the second-order region were 300 nm, which were chosen based on the Bragg condition:

mλ_{Bragg}+2n_{eff}Λₘ

where m is the order of diffraction, λ_{Bragg} is the Bragg wavelength, and n_{eff} is the effective refractive index of the gain medium.

The DFB substrates were cleaned by conventional ultrasonication. A chloroform solution of Compound 25 and 4,4′-bis(N-carbazolyl)-1,10-biphenyl (CBP) (weight ratio, 6:94) was spin-coated on top of the DFB substrates to form a light-emitting layer of 240 nm thick. A 2 μm thick CYTOP polymer layer was directly formed on top the structure by spin-coating and then covered by a sapphire lid with a thermal conductivity of 25 W m⁻¹ K⁻¹ at 300 K to fabricate a second-order DFB laser with the structure glass/SiO₂/6 wt % Compound 25:CBP/CYTOP/sapphire lid.

The PLQY of the thin film of Compound 25 (6 wt %) and CBP was 85%. The PL spectra and the transient PL decay curve of thin film were obtained. The exciton lifetime was estimated to be 2.5 ns.

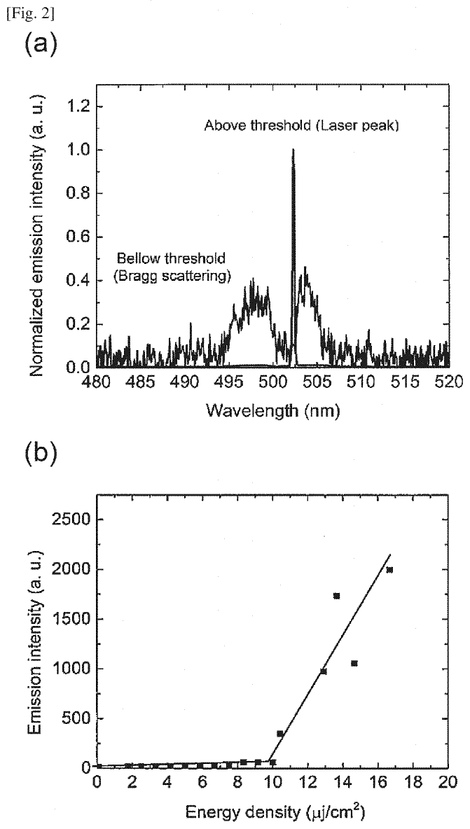

The performance of the DFB laser was characterized using a nitrogen-gas laser pump. The excitation wavelength of the pump was 337 nm, the pulse width was 3.5 ns and the repetition rate was 20 Hz. FIGS. 2(a) and 2(b) show the emission spectrum of the DFB laser and the emission output intensity as a function of the pump energy density, respectively. The threshold was found to be 10 μJ/cm² and the FWHM was 0.2 nm.

### (Example 3) Yellow Optically Pumped Distributed Feedback (DFB) Organic Laser

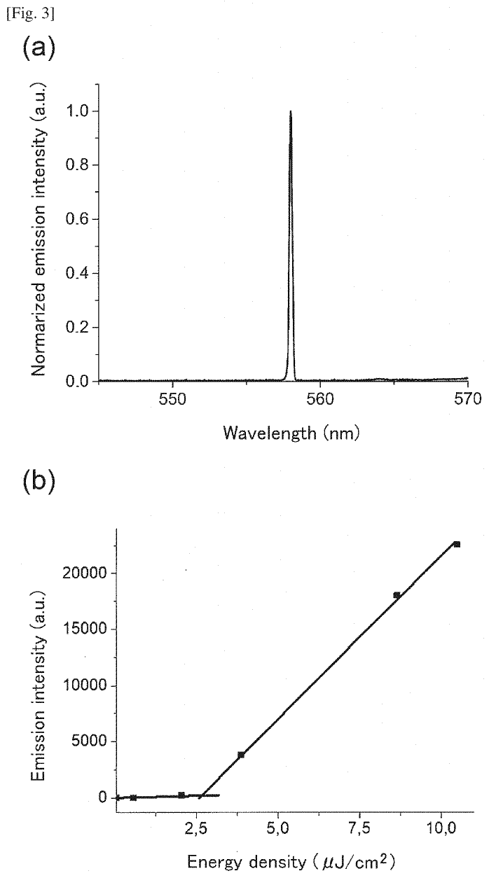

A second order distributed feedback (DFB) organic laser was fabricated on glass in the same method as Example 2. The period of the grating was 360 nm. A chloroform solution of neat Compound 7 was spin coated on top of the DFB grating to form a light emitting layer. The obtained thickness was 220 nm. A few m thick Cytop polymer layer was spin coated on top of the structure and then covered by Sapphire lid. The performance of the DFB laser was characterized using a nitrogen-gas laser pump. The excitation wavelength of the pump was 337 nm, the pulse width was 3.5 ns and the repetition rate was 20 Hz. FIGS. 3(a) and 3(b) show the emission spectrum of the DFB laser and the emission output intensity as a function of the pump energy density. The threshold was found to be 2.6 μJ/cm² and the FWHM was 0.6 nm.

### (Example 4) Other Optically Pumped Distributed Feedback (DFB) Organic Laser

A second order distributed feedback (DFB) organic laser was fabricated on glass in the same method as Example 2. The period of the grating was 300 nm. A chloroform solution of a mixture of CBP and Compound 6 was spin coated on top of the DFB grating to form a light emitting layer. A few m thick Cytop polymer layer was spin coated on top of the structure and then covered by Sapphire lid.

Other optically pumped DFB organic lasers were manufactured in the same manner except that Compounds 2, 5, 7, 17, 22, 23, 26, 28, 31, 32, 34, 43 to 46 and 49 were used instead of Compound 6, and the periods were changed as shown in Table 2.

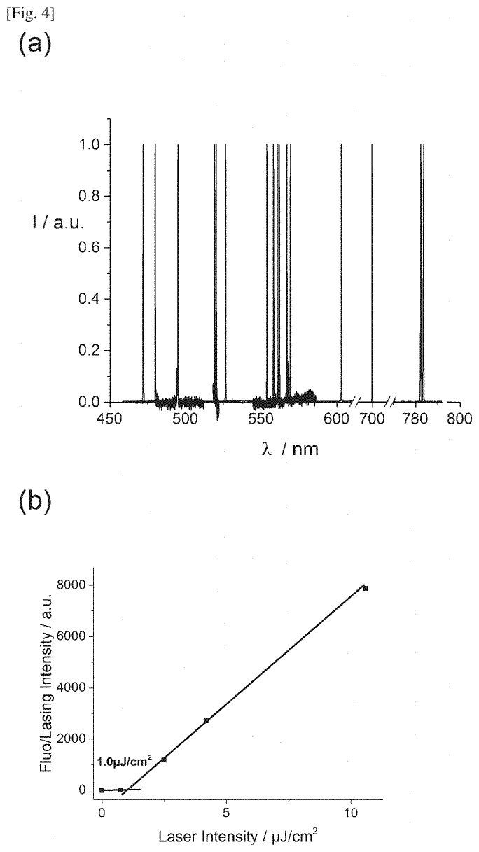

The performance of the DFB lasers were characterized using a nitrogen-gas laser pump. The excitation wavelength of the pump was 337 nm, the pulse width was 3.5 ns and the repetition rate was 20 Hz. FIG. 4(a) shows the emission spectrum of the DFB lasers. As is clear from the spectrum, FWHM of each DFB laser was very small. FIG. 4(b) shows the emission output intensity of the DFB laser using Compound 6 as a function of the pump energy density.

TABLE 2

| | ^{λ}lasing | Threshold | Period |
|---|---|---|---|
| Compound | [nm] | [μJ/cm²] | [nm] |
| Compound 2 | 553.8 | 9.6 | 360 |
| Compound 5 | 471.9 | 1.4 | 290 |
| Compound 6 | 480.0 | 1.0 | 300 |
| Compound 7 | 558.0 | 2.6 | 360 |
| Compound 17 | 569.5 | 3.4 | 370 |
| Compound 22 | 559.0 | 2.4 | 360 |
| Compound 23 | 567.0 | 3.8 | 370 |
| Compound 26 | 526.9 | 4.7 | 330 |
| Compound 28 | 561.0 | 1.8 | 360 |
| Compound 31 | 519.2 | 3.9 | 330 |
| Compound 32 | 603.0 | 1.55 | 390 |
| Compound 34 | 782.4 | 10.0 | 510 |
| Compound 43 | 520.3 | 10.2 | 330 |
| Compound 44 | 495.0 | 3.3 | 310 |
| Compound 45 | 783.5 | 3.4 | 510 |
| Compound 46 | 569.2 | 8.0 | 370 |
| Compound 49 | 699.7 | 3.7 | 450 |

1. An organic solid-state laser containing a compound represented by the following formula (1): 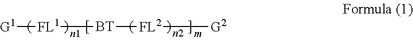
wherein:
G¹ and G² each independently represent a hydrogen atom or a substituent;
FL¹ and FL² each independently represent a linking group represented by the following formula (2) or formula (3): 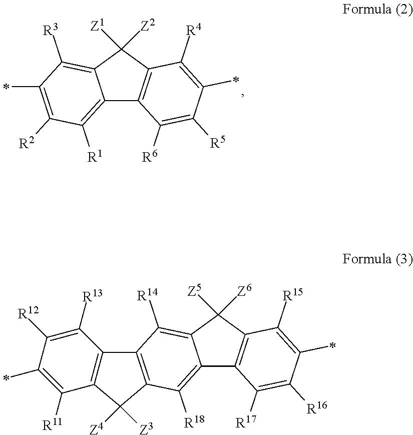
wherein R¹ to R⁶, R¹¹ to R¹⁸ and Z¹ to Z⁶ each independently represent a hydrogen atom or a substituent, R¹ and R², R⁵ and R⁶, R¹² and R¹³, and R¹⁶ and R¹⁷ may be bonded together to form a ring, and each * represents a bonding site;
BT represents a linking group represented by the following formula (4) or formula (5): 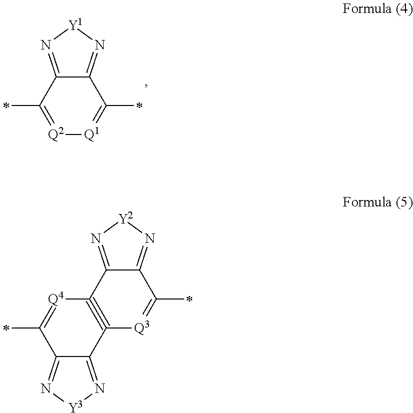
wherein Y¹ represents an oxygen atom, a sulfur atom or N-T¹, Y² represents an oxygen atom, a sulfur atom or N-T², Y³ represents an oxygen atom, a sulfur atom or N-T³, Q¹ represents a nitrogen atom or C—X¹, Q² represents a nitrogen atom or C—X², Q³ represents a nitrogen atom or C—X³, Q⁴ represents a nitrogen atom or C—X⁴, T¹ to T³ each independently represent a substituted or unsubstituted alkyl group, X¹ to X⁴ each independently represent a hydrogen atom, a substituted or unsubstituted alkyl group, a substituted or unsubstituted alkoxy group or a halogen atom, or X¹ and X² may be bonded together to form a ring, and each * represents a bonding site;
n1, n2 and m each independently represent an integer of from 1 to 5;
rightmost n2 may be 0; and when m is an integer of from 2 to 5 and rightmost n2 is 0, then n1 may be 0;
when m is two or more, then two or more instances of BT may be the same or different, two or more instances of FL² may be the same or different, and two or more instances of n2 may be the same or different;
when n1 is two or more, then two or more instances of FL¹ may be the same or different; and
when n2 is two or more, then two or more instances of FL² may be the same or different.

2. The organic solid-state laser according to claim 1, wherein BT is a linking group represented by the formula (4).

3. The organic solid-state laser according to claim 1, wherein BT is a linking group represented by the formula (5).

4. The organic solid-state laser according to claim 2, wherein BT is a linking group represented by the following formula (4a): 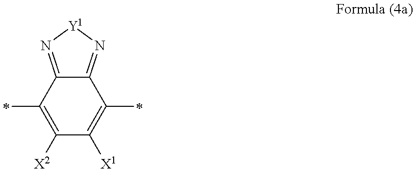
wherein Y¹ represents an oxygen atom, a sulfur atom or N-T¹, T¹ represents a substituted or unsubstituted alkyl group, X¹ and X² each independently represent a hydrogen atom, a substituted or unsubstituted alkyl group, a substituted or unsubstituted alkoxy group or a halogen atom, or X¹ and X² may be bonded together to form a ring, and each * represents a bonding site.

5. The organic solid-state laser according to claim 4, wherein BT is a linking group represented by the following formula (4b): 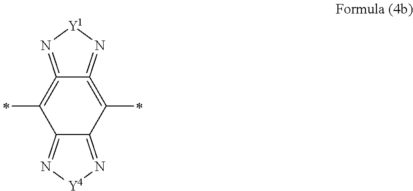
wherein Y¹ represents an oxygen atom, a sulfur atom or N-T¹, Y⁴ represents an oxygen atom, a sulfur atom or N-T⁴, T¹ and T⁴ each independently represent a substituted or unsubstituted alkyl group, and each * represents a bonding site.

6. The organic solid-state laser according to claim 4, wherein BT is a linking group represented by the following formula (4c): 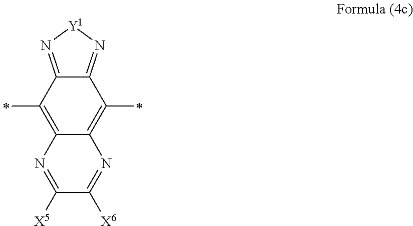
wherein Y¹ represents an oxygen atom, a sulfur atom or N-T¹, T¹ represent a substituted or unsubstituted alkyl group, X⁵ and X⁶ each independently represent a hydrogen atom a substituted or unsubstituted alkyl group, X⁵ and X⁶ can be bonded together to form a ring, and each * represents a bonding site.

7. The organic solid-state laser according to claim 3, wherein BT is a linking group represented by the following formula (5a): 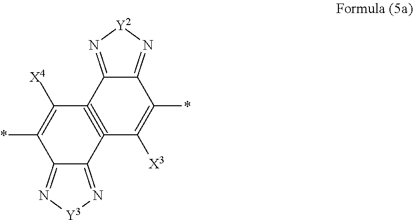
wherein Y² represents an oxygen atom, a sulfur atom or N-T², Y³ represents an oxygen atom, a sulfur atom or N-T³, T² and T³ each independently represent a substituted or unsubstituted alkyl group, X³ and X⁴ each independently represent a hydrogen atom, a substituted or unsubstituted alkyl group, a substituted or unsubstituted alkoxy group or a halogen atom, and each * represents a bonding site.

8. The organic solid-state laser according to claim 1, wherein BT is each independently selected from the group consisting of the following BT1 to BT12: 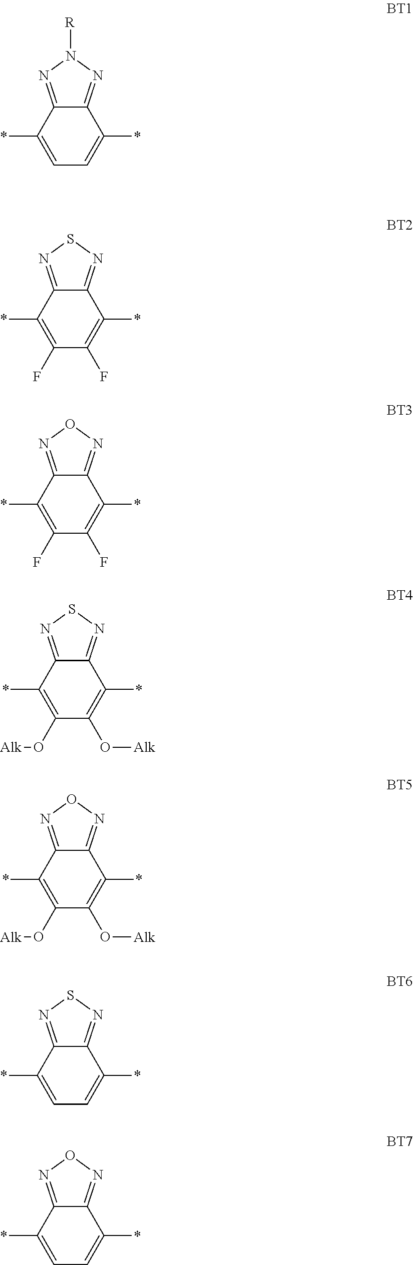 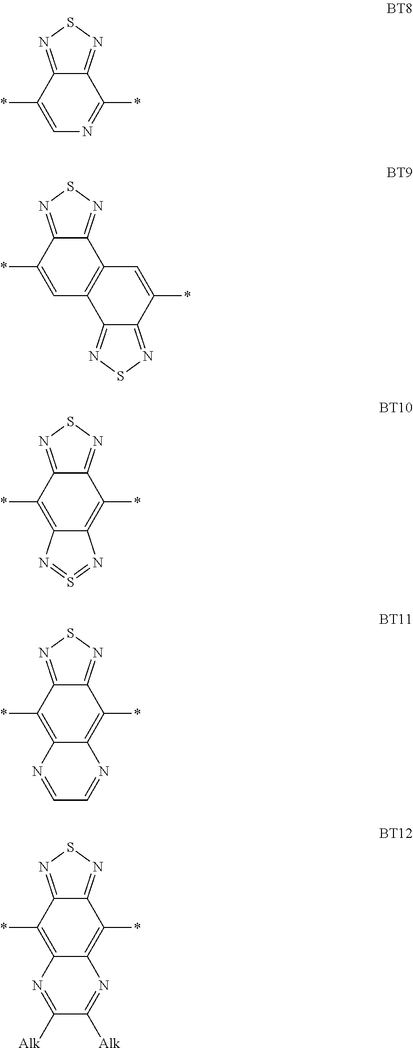
wherein R and Alk each independently represent a substituted or unsubstituted alkyl group and each * represents a bonding site.

9. The organic solid-state laser according to claim 1, wherein FL¹ and FL² are each independently a linking group represented by the formula (2).

10. The organic solid-state laser according to claim 1, wherein FL¹ and FL² are each independently a linking group represented by the formula (3).

11. The organic solid-state laser according to claim 1, wherein Z¹ to Z⁶ each independently represent a substituted or unsubstituted alkyl group.

12. The organic solid-state laser according to claim 1, wherein R¹ to R⁶ and R¹¹ to R¹⁸ are a hydrogen atom.

13. The organic solid-state laser according to claim 1, wherein G¹ and G² are each independently a substituted or unsubstituted aryl group or a substituted or unsubstituted diarylamino group.

14. The organic solid-state laser according to claim 1, wherein the compound is represented by the following formula (1a): 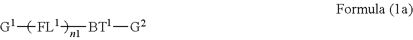
wherein G¹ and G² each independently represent a hydrogen atom or a substituent, FL¹ represents a linking group represented by the formula (2) or formula (3), BT¹ represents a linking group represented by the formula (4) or formula (5), and n1 is an integer of from 1 to 5.

15. The organic solid-state laser according to claim 1, wherein the compound is represented by the following formula (1b): 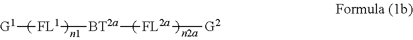
wherein G¹ and G² each independently represent a hydrogen atom or a substituent, FL¹ and FL^{2a} each independently represent a linking group represented by the formula (2) or formula (3), BT^{2a} represents a linking group represented by the formula (4) or formula (5), and n1 and n2a are each independently an integer of from 1 to 5.

16. The organic solid-state laser according to claim 1, wherein the compound is represented by the following formula (1c): 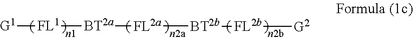
wherein G¹ and G² each independently represent a hydrogen atom or a substituent, FL¹, FL^{2a} and FL^{2b} each independently represent a linking group represented by the formula (2) or formula (3), BT^{2a} and BT²b each independently represent a linking group represented by the formula (4) or formula (5), n1 and n2a are each independently an integer of from 1 to 5, and n2b is an integer of from 0 to 5.

17. The organic solid-state laser according to claim 1, wherein the compound is represented by the following formula (1d): 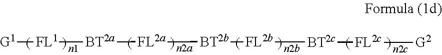
wherein G¹ and G² each independently represent a hydrogen atom or a substituent, FL¹, FL^{2a}, FL^{2b} and FL^{2c} each independently represent a linking group represented by the formula (2) or formula (3), BT^{2a}, BT^{2b} and BT^{2c} each independently represent a linking group represented by the formula (4) or formula (5), n1, n2a and n2b are each independently an integer of from 1 to 5, and n2c is an integer of from 0 to 5.

18. The organic solid-state laser according to claim 1, wherein the compound is represented by the following formula (1e): 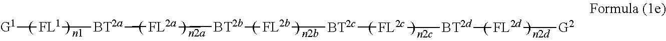
wherein G¹ and G² each independently represent a hydrogen atom or a substituent, FL¹, FL^{2a}, FL^{2b}, FL^{2c} and FL^{2d} each independently represent a linking group represented by the formula (2) or formula (3), BT^{2a}, BT^{2b}, BT^{2c} and BT^{2d} each independently represent a linking group represented by the formula (4) or formula (5), n1, n2a, n2b and n2c are each independently an integer of from 1 to 5, and n2d is an integer of from 0 to 5.

19. The organic solid-state laser according to claim 1, wherein the compound is represented by the following formula (1f): 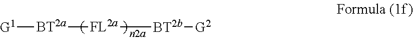
wherein, G¹ and G² each independently represent a hydrogen atom or a substituent, FL^{2a} represents a linking group represented by the formula (2) or formula (3), BT^{2a} and BT^{2b} each independently represent a linking group represented by the formula (4) or formula (5), and n2a is an integer of from 1 to 5.

20. The organic solid-state laser according to claim 1, wherein the compound is represented by the following formula (1g): 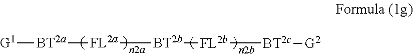
wherein G¹ and G² each independently represent a hydrogen atom or a substituent, FL^{2a} and FL^{2b} each independently represent a linking group represented by the formula (2) or formula (3), BT^{2a}, BT^{2b} and BT^{2c} each independently represent a linking group represented by the formula (4) or formula (5), and n2a and n2b are each independently an integer of from 1 to 5.

21. The organic solid-state laser according to claim 1, wherein the compound is represented by the following formula (1h): 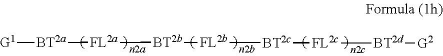
wherein G¹ and G² each independently represent a hydrogen atom or a substituent, FL^{2a}, FL^{2b} and FL^{2e} each independently represent a linking group represented by the formula (2) or formula (3), BT^{2a}, BT^{2b}, BT^{2c} and BT^{2d} each independently represent a linking group represented by the formula (4) or formula (5), and n2a, n2b and n2c are each independently an integer of from 1 to 5.

22. A compound represented by the following formula (1): 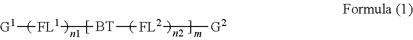
wherein:
G¹ and G² each independently represent a hydrogen atom or a substituent;
FL¹ and FL² are each independently a linking group represented by the following formula (2) or formula (3): 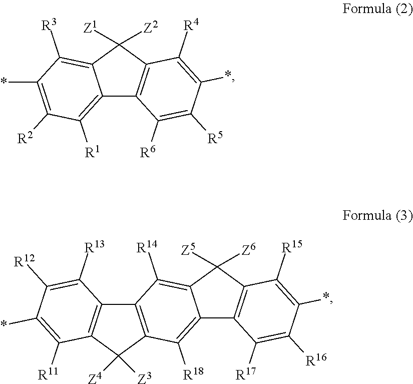
wherein R¹ to R⁶, R¹¹ to R¹⁸ and Z¹ to Z⁶ each independently represent a hydrogen atom or a substituent, R¹ and R², R⁵ and R⁶, R¹² and R¹³, and R¹⁶ and R¹⁷ may be bonded together to form a ring, and each * represents a bonding site;
BT represents a linking group represented by the following formula (4) or formula (5): 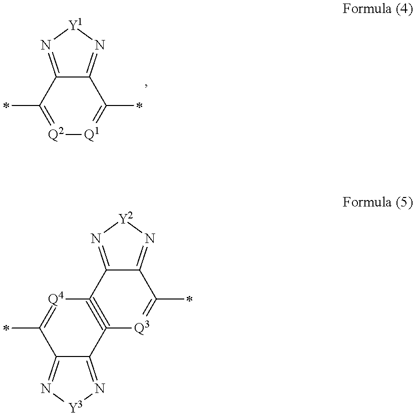
wherein Y¹ represents an oxygen atom, a sulfur atom or N-T¹, Y² represents an oxygen atom, a sulfur atom or N-T², Y³ represents an oxygen atom, a sulfur atom or N-T³, Q¹ represents a nitrogen atom or C—X¹, Q² represents a nitrogen atom or C—X², Q³ represents a nitrogen atom or C—X³, Q⁴ represents a nitrogen atom or C—X⁴, T¹ to T³ each independently represent a substituted or unsubstituted alkyl group, X¹ to X⁴ each independently represent a hydrogen atom, a substituted or unsubstituted alkyl group, a substituted or unsubstituted alkoxy group or a halogen atom, or X¹ and X² may be bonded together to form a ring, and each * represents a bonding site;
n1, n2 and m each independently represent an integer of from 1 to 5;
rightmost n2 may be 0;
when m is two or more, then two or more instances of BT may be the same or different, two or more instances of FL² may be the same or different, and two or more instances of n2 may be the same or different;
when n1 is two or more, then two or more instances of FL¹ may be the same or different;
when n2 is two or more, then two or more instances of FL² may be the same or different,
when n1 and m are 1, then at least one of the following three conditions are satisfied:
(1) at least one of FL¹ and FL² is a linking group represented by the formula (3),
(2) BT is a linking group represented by the formula (5), and
(3) at least one of G¹ and G² is a substituted or unsubstituted diarylamino group.

23. (canceled)
